(19) United States
(12) Patent Application Publication
(10) Pub. No.: US 2004/0260318 A1
(43) Pub. Date: Dec. 23, 2004

(54) Anastomotic connector devices

(75) Inventors: William L. Hunter, Vancouver (CA); Philip M. Toleikis, Vancouver (CA); David M. Gravett, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Angiotech International AG, Zug (CH)

(21) Appl. No.: 10/853,023

(22) Filed: May 24, 2004

Related U.S. Application Data
(60) Provisional application No. 60/473,185, filed on May 23, 2003.
(60) Provisional application No. 60/523,908, filed on Nov. 20, 2003.
(60) Provisional application No. 60/525,226, filed on Nov. 24, 2003.
(60) Provisional application No. 60/526,541, filed on Dec. 3, 2003.

(51) Int. Cl. A61F 2/06

(52) U.S. Cl. 606/153; 623/1.42

(57) ABSTRACT

Anastomotic connector devices are provided which release a therapeutic agent. The therapeutic agent may be an anti-scarring agent that inhibits stenosis caused by the presence of the anastomotic connector device.

### CROSS-REFERENCE TO RELATED APPLICATION

This application claims the benefit of U.S. Provisional Patent Application No. 60/473,185 filed May 23, 2003, where this provisional application is incorporated herein by reference in its entirety.

### BACKGROUND OF THE INVENTION

1. Field of the Invention The present invention relates generally to pharmaceutical compositions, methods, and devices, and more specifically, to anastomotic connector devices which release a desired therapeutic agent.

2. Description of the Related Art

The creation of vascular anastomoses has been an integral part of vascular surgery since the advent of bypass surgery in the early 20^{th} century. Briefly, bypass is the creation of an alternative conduit to permit blood to flow around an obstructed (or damaged) artery. Most commonly, this occurs when an artery (typically a coronary artery, carotid artery, or artery supplying the lower limb) becomes completely or partially obstructed by atherosclerosis (plaque) or clot, leading to ischemia of the tissues supplied by the artery. In an attempt to restore blood flow to the affected region, one end of the conduit (typically one of the patient's own arteries or veins harvested from a different site or alternatively, a synthetic vascular graft) is inserted into the vasculature proximal to the obstruction (“upstream”) and the other end is inserted distal (“downstream”) to the obstruction to provide an alternative route for blood to reach the ischemic tissue. The connection of the conduit to the native vasculature is referred to as an “anastomosis;”and are further described as being either “proximal” or “distal” depending on its location relative to the vascular obstruction. Traditionally, vascular anastomoses have been created by a vascular or cardiac surgeon suturing each end of the conduit (e.g., a saphenous vein, internal mammary artery, a synthetic vascular graft) in place during an open surgical procedure. Suturing anastomoses by hand is time consuming (each anastomosis requires approximately 5-10 minutes to complete depending on location) and results in an increase in the time the patient is in surgery, under anaesthesia, and/or connected to heart/lung bypass.

Recently, sutureless anastomotic devices have been created to mechanize the creation of a vascular anastomosis. Although there are numerous types and designs of anastomotic devices, all are designed to produce a facilitated semiautomatic vascular anastomosis without the use of suture and reduce connection time substantially (often to several seconds). The ideal anastomotic device is reproducible every time, creates a round and smooth anastomosis, has strength and sealing equal to sutures, eliminates the need for aortic cross-clamping and cardiopulmonary bypass (in coronary artery bypass grafting—CABG), and reduces procedural time. Any device capable of achieving one or more of these characteristics would qualify as an anastomotic device.

Despite recent advances in the construction of anastomotic connector devices, there is a need in the art for new anastomotic devices, which can release a desired therapeutic agent, which can alleviate, reduce, and/or inhibit problems associated with the use of anastomotic connector devices. The present invention discloses such devices (as well as compositions and methods for making such devices) and, further, provides other related advantages.

### BRIEF SUMMARY OF THE INVENTION

Briefly stated, the present invention provides anastomotic connector devices, which release a desired therapeutic agent.

For example, in one aspect the invention provides an anastomotic coupling device comprising (i) an anastomotic coupling device and (ii) an anti-scarring agent. The agent is associated with the device in a manner that provides for the sustained release of the agent from the device when the device is inserted into a patient. The agent is released from the device at a therapeutically effective rate that inhibits stenosis.

Within representative embodiments, the therapeutic agent is an anthracycline (e.g., doxorubicin or mitoxantrone), a taxane (paclitaxel or docetaxel), an immunosuppressant such as sirolimus, or a sirolimus analogue (e.g., everolimus), and/or a podophyllotoxin (e.g., etoposide). Within various embodiments of the invention the desired therapeutic agent is admixed with and/or released from a carrier, such as, for example, a polymer. Within yet other embodiments of the invention, the anastomotic connector device further comprises (or may alternatively comprise) an anti-thrombogenic and/or anti-platelet agent (e.g., heparin).

Within other aspects of the present invention methods are provided for creating a pathway between two vascular structures, or between two different parts of the same vascular structure, comprising the step of introducing an anastomotic connector device into a patient where it is desired to create a pathway between two vascular structures, or between two different parts of the same vascular structure, wherein the anastomotic connector device is one of the devices which release a therapeutic agent as described herein. Utilizing such methods a pathway or anastomosis can be created between a variety of vascular structures, including: artery-to-artery, vein-to-artery, artery-to-vein, artery-to-synthetic graft, synthetic graft-to-artery, vein-to-synthetic graft or synthetic graft-to-vein.

Within yet other aspects of the present invention methods are provided for making anastomotic connector devices, comprising coating all or a portion of an anastomotic device with an anthracycline, taxane, an immunosuppressant such as sirolimus, or a sirolimus analogue, or a podophyllotoxin or other anti-restenotic agents. Within various embodiments, the device can be coated with a desired therapeutic agent by spraying or dipping the device with the agent.

Within further aspects of the invention, anastomotic connector devices are provided which are coated, or otherwise adapted to release an anti-thrombogenic and/or anti-platelet agent such as heparin. Anastomotic connector devices are also provided with a coating, which enhances its echogenicity (i.e., visualization under ultrasound).

These and other aspects of the present invention will become evident upon reference to the following detailed description and attached drawings. In addition, various references are set forth herein which describe in more detail certain procedures or compositions (e.g., devices, compositions, compounds or agents and methods for making such devices, compositions, compounds or agents, etc.), and are therefore incorporated by reference in their entirety. When PCT applications are referred to it is also understood that the underlying or cited U.S. applications are also incorporated by reference herein in their entirety.

### BRIEF DESCRIPTION OF THE DRAWINGS

FIG. 1 is a diagram showing how a Cell Cycle Inhibitor acts at one or more of the steps in the biological pathway.

FIG. 2 is graph showing the results of a screening assay for assessing the effect of Mitoxantrone on cell proliferation (Mitoxantrone IC₅₀=20 nM for proliferation of human fibroblasts).

FIG. 3 is a picture that shows an uninjured carotid artery from a rat balloon injury model.

FIG. 4 is a picture that shows an injured carotid artery from a rat balloon injury model.

FIG. 5 is a picture that shows a paclitaxel/mesh treated carotid artery in a rat balloon injury model (345 μg paclitaxel in a 50:50 PLG coating on a 10:90 PLG mesh).

FIG. 6A schematically depicts the transcriptional regulation of matrix metalloproteinases. FIG. 6B is a blot which demonstrates that IL-1 stimulates AP-1 transcriptional activity.

FIG. 7A-H are blots which show the effect of various anti-microtubule agents in inhibiting collagenase expression.

FIG. 8 is a graph showing the results of a screening assay for assessing the effect of Paclitaxel on smooth muscle cell migration (Paclitaxel IC₅₀=0.76 nM).

FIG. 9 is a graph showing the results of a screening assay for assessing the effect of geldanamycin on IL-1β production by macrophages (IC₅₀=20 nM for IL-1β production by THP-1 cells).

FIG. 10 is a graph showing the results of a screening assay for assessing the effect of geldanamycin on IL-8 production by macrophages (IC₅₀=27 nM for IL-8 production by THP-1 cells).

FIG. 11 is a graph showing the results of a screening assay for assessing the effect of geldanamycin on MCP-1 production by macrophages (IC₅₀=7 nM for MCP-1 production by THP-1 cells).

### DETAILED DESCRIPTION OF THE INVENTION

Prior to setting forth the invention, it may be helpful to an understanding thereof to set forth definitions of certain terms that will be used hereinafter.

“Anastomosis” refers to a direct or indirect communication or connection between two or more normally separate spaces or organs (e.g., blood vessels). The term also refers to a passageway, created by, for example, surgery, between two blood vessels or other blood-containing or blood-carrying structures.

“Anasomotic connector device” refers to any vascular device that mechanizes the creation of a vascular anastomosis (i.e., artery-to-artery, vein-to-artery, artery-to-vein, artery-to-synthetic graft, synthetic graft-to-artery, vein-to-synthetic graft or synthetic graft-to-vein anastomosis) without the manual suturing that is typically done in the creation of an anastomosis. The term also refers to anastomotic connector devices (described below), designed to produce a facilitated semiautomatic vascular anastomosis without the use of suture and reduce connection time substantially (often to several seconds), where there are numerous types and designs of such devices. The term also refers to devices which facilitate attachment of a vascular graft to an aperature or orifice (e.g., in the side or at the end of a vessel) in a target vessel. Anastomotic connector devices may be anchored to the outside of a blood vessel, and/or into the wall of a blood vessel (e.g., into the adventitial, intramural, or intimal layer of the tissue), and/or a portion of the device may reside within the lumen of the vessel.

Anastomotic connector devices also may be used to create new flow from one structure to another through a channel or diversionary shunt. Accordingly, such devices (also referred to herein as “bypass devices”) typically include at least one tubular structure, wherein a tubular structure defines a lumen. Anastomotic connector devices may include one tubular structure or a plurality of tubular structures through which blood can flow. At least a portion of the tubular structure resides external to a blood vessel (i.e., extravascular) to provide a diversionary passageway. A portion of the device also may reside within the lumen and/or within the tissue of the blood vessel.

The ideal anastomotic connector device is reproduceable, creates a round and smooth anastomosis, has strength and sealing equal to (or superior to) sutures, reduces the need for aortic cross-clamping and cardiopulmonary bypass, as well as procedural time. Any device capable of achieving one or more of these characteristics would qualify as an anastomotic connector device.

“Fibrosis” or “Scarring” refers to the formation of fibrous tissue in response to injury or medical intervention. Therapeutic agents which inhibit fibrosis or scarring can do so through one or more mechanisms including: inhibiting angiogenesis, inhibiting migration or proliferation of connective tissue cells (e.g., fibroblasts, smooth muscle cells, vascular smooth muscle cells), and/or immune inflammatory cells (e.g. macrophages, lymphocytes, neutrophils), reducing ECM production, and/or inhibiting tissue remodeling. In addition, numerous therapeutic agents that are described will have the additional benefit of also reducing tissue regeneration (the replacement of injured cells by cells of the same type) when appropriate. Furthermore, numerous therapeutic agents will have the additional benefit of inhibiting inflammatory processes (e.g. pro-inflammatory cytokine production, pro-inflammatory chemokine production).

In the present description, any concentration range, percentage range, ratio range, or integer range is to be understood to include the value of any integer within the recited range and, when appropriate, fractions thereof (such as one tenth and one hundredth of an integer, etc.), unless otherwise indicated. As used herein, “about” or “comprising essentially of” mean ±15%. As used herein, the use of an indefinite article, such as “a” or “an”, should be understood to refer to the singular and the plural of a noun or noun phrase (i.e., meaning “one or more” of the enumerated elements or components). The use of the alternative (e.g., “or”) should be understood to mean either one, both or any combination thereof of the alternatives. In addition, it should be understood that the individual compounds, or groups of compounds, derived from the various combinations of the sequences, structures, and substituents described herein, are disclosed by the present application to the same extent as if each compound or group of compounds was set forth individually. Thus, selection of particular sequences, structures, or substituents is within the scope of the present invention.

As noted above, the present invention provides anastomotic connector devices which are adapted to release a desired therapeutic agent. A preferred agent according to the present invention is an anti-scarring agent. Although presently available anastomotic connector devices have the potential to dramatically improve vascular surgery by reducing procedural time and eliminating morbidity associated with cross-clamping the aorta (an intervention known to cause embolic strokes in the brain) and use of cardiopulmonary bypass (known to contribute to brain ischemia and cognitive impairment), they do not eliminate, and may contribute to, the problem of post-procedural anastomotic stenosis. In particular, injury to the graft tissue or recipient artery during surgery and/or injury created by the change in blood flow pattern as a result of the anastomosis leads to a well-known clinical problem called restenosis (or in this circumstance, it can also correctly be referred to as a “stenosis”). Restenosis occurs in response to vascular reconstructive procedures, including virtually any manipulation or perturbation of the natural state of the tissue, which attempts to relieve vessel obstructions, and is a major factor limiting the effectiveness of invasive treatments for vascular diseases.

Restenosis is a form of vascular wall response to injury leading to vessel wall thickening and loss of blood flow to the tissue supplied by the artery. Injury to the vascular wall results in damage to endothelial and smooth muscle cells (SMCs) that release cytokines, which recruit inflammatory cells such as macrophages, lymphocytes and neutrophils (i.e., which are some of the known white blood cells) into the area. The white blood cells in turn release a variety of cytokines, growth factors, and tissue degrading enzymes that influence the behaviour of the constituent cells of the vascular wall (primarily endothelial cells and SMCs). Stimulation of the vascular SMCs induces them to migrate into the inner aspect of the vessel (the intima), proliferate and secrete an extracellular matrix. Collectively, this creates a thickening of the intimal layer (known as neointimal hyperplasia) that narrows the lumen of the vessel and can be significant enough to obstruct blood flow.

As more and more vascular bypass procedures are performed using anastomotic connector devices, it will become increasingly imperative to create devices with reduced rates of stenosis/restenosis. The present invention provides anastomotic connector devices which are adapted to release a therapeutic agent and thereby inhibit stenosis from occuring. The devices are “adapted” in the sense that they contain the therapeutic agent in such a manner that the agent stays in association with the device prior to surgery, but then after the device has been inserted into the host, the device will elute or otherwise release agent such that the agent is no longer associated with the device and thereby interacts with local tissue or distant tissue. In order to further an understanding of devices according to the present invention, the following are discussed in more detail below: (I) agents; (II) compositions that contain the agent; and (III) anastomotic Connector Devices.

Briefly, a wide variety of agents (also referred to herein as ‘therapeutic agents’ or ‘drugs’) can be utilized within the context of the present invention. The agent may be formulated with one or more other materials, e.g., a polymeric carrier, where these formulations are discussed later herein. Many suitable therapeutic agents are specifically identified herein, and others may be readily determined based upon in vitro and in vivo (animal) models, such as those provided in Examples 21-31. For example, a preferred therapeutic agent is an agent which can inhibit fibrosis, also known as an anti-scarring agent, wherein exemplary anti-scarring agents include agents that are anti-proliferative, and/or that inhibit cell migration, and/or that inhibit inflammation, and/or that are anti-angiogenic.

The assay set forth in Example 21 may be used to determine whether an agent is able to inhibit cell proliferation in fibroblasts and/or smooth muscle cells. In one aspect of the invention, the agent has an IC₅₀ for inhibition of cell proliferation within a range of about 10⁻⁶ to about 10⁻¹⁰M. The assay set forth in Example 28 may be used to determine whether an agent may inhibit migration of fibroblasts and/or smooth muscle cells. In one aspect of the invention, the agent has an IC₅₀ for inhibition of cell migration within a range of about 10⁻⁶ to about 10⁻⁹M. Assays set forth herein may be used to determine whether an agent is able to inhibit inflammatory processes, including nitric oxide production in macrophages (Example 22), and/or TNF-alpha production by macrophages (Example 23), and/or IL-1 beta production by macrophages (Example 29), and/or IL-8 production by macrophages (Example 30), and/or inhibition of MCP-1 by macrophages (Example 31). In one aspect of the invention, the agent has an IC₅₀ for inhibition of any one of these inflammatory processes within a range of about 10⁻⁶ to about 10⁻¹⁰M. The assay set forth in Example 26 may be used to determine whether an agent is able to inhibit MMP production. In one aspect of the invention, the agent has an IC₅₀ for inhibition of MMP production within a range of about 10⁻⁴ to about 10⁻⁸M. The assay set forth in Example 27 (also known as the CAM assay) may be used to determine whether an agent is able to inhibit angiogenesis. In one aspect of the invention, the agent has an IC₅₀ for inhibition of angiogenesis within a range of about 10⁻⁶ to about 10⁻¹⁰M. Agents which inhibit fibrosis can also be identified through in vivo models including inhibition of intimal hyperplasia development in the rat balloon carotid artery model (Example 25) and/or a reduction of surgical adhesions formation in rabbit surgical adhesions model (Example 24).

Numerous therapeutic compounds have been identified that are of utility in the present invention including the following:

A. 5-Lipoxygenase Inhibitors & Antagonists

In one embodiment of the present invention, the pharmacologically active compound associated with the anastomotic connector device is a 5-lipoxygenase inhibitor or antagonist. Exemplary compounds having this biological activity include the following, where the present invention provides that each of these specifically named compounds may be placed in association with an anastomotic connection device of the present invention: Wy-50295 (2-Naphthaleneacetic acid, Alpha-methyl-6-(2-quinolinylmethoxy)-, (S)-), ONO-LP- 269 (2,11,14-Eicosatrienamide, N-[4-hydroxy-2-(1H-tetrazol-5-yl)-8-quinolinyl]-, (E,Z,Z)-), licofelone (1H-Pyrrolizine-5-acetic acid, 6-(4-chlorophenyl)-2,3-dihydro-2,2-dimethyl-7-phenyl-), CMI-568 (Urea, N-butyl-N-hydroxy-N′-[4-[3-(methylsulfonyl)-2-propoxy-5-[tetrahydro-5-(3,4,5-trimethoxyphenyl)-2-furanyl]phenoxy]butyl]-,trans-), IP-751 ((3R,4R)-(delta6)-THC-DMH-11-oic acid), PF-5901 (Benzenemethanol, Alpha-pentyl-3-(2-quinolinylmethoxy)-), LY-293111 (Benzoic acid, 2-[3-[3-[(5-ethyl-4′-fluoro-2-hydroxy[1,1′-biphenyl]-4-yl)oxy]propoxy]-2-propylphenoxy]-), RG-5901 -A (Benzenemethanol, Alpha-pentyl-3-(2-quinolinylmethoxy)-, hydrochloride), rilopirox (2(1H)-Pyridinone, 6-[[4-(4-chlorophenoxy)phenoxy]methyl]-1-hydroxy-4-methyl-), L-674636 (Acetic acid, ((4-(4-chlorophenyl)-1-(4-(2-quinolinylmethoxy)phenyl)butyl)thio)-AS]), 7-[[3-(4-methoxy-tetrahydro-2H-pyran-4-yl)phenyl]methoxy]-4-phenylnaphtho[2,3-c]furan-1(3H)-one, MK-886 (1H-Indole-2-propanoic acid,1-[(4-chlorophenyl)methyl]-3-[(1,1-dimethylethyl)thio]-Alpha,Alpha-dimethyl-5-(1-methylethyl)-), quiflapon (1H-Indole-2-propanoic acid,1-[(4-chlorophenyl)methyl]-3-[(1,1-dimethylethyl)thio]-Alpha,Alpha-dimethyl-5-(2-quinolinylmethoxy)-), quiflapon (1H-Indole-2-propanoic acid,1-[(4-chlorophenyl)methyl]-3-[(1,1-dimethylethyl)thio]-Alpha,Alpha-dimethyl-5-(2-quinolinylmethoxy)-), docebenone (2,5-Cyclohexadiene-1,4-dione, 2-(12-hydroxy-5,10-dodecadiynyl)-3,5,6-trimethyl-), zileuton (Urea, N-(1-benzo[b]thien-2-ylethyl)-N-hydroxy-, or an analogue or derivative thereof.

B. Chemokine Receptor Antagonists CCR (1, 3, & 5)

In one embodiment of the present invention, the pharmacologically active compound associated with the anastomotic connector device is a chemokine receptor antagonist. Exemplary compounds having this biological activity include the following, where the present invention provides that each of these specifically named compounds may be placed in association with an anastomotic connection device of the present invention: ONO-4128 (1,4,9-Triazaspiro(5,5)undecane-2,5-dione,1-butyl-3-(cyclohexylmethyl)-9-((2,3-dihydro-1,4-benzodioxin-6-yl)methyl-), L-381, CT-112 (L-Arginine, L-threonyl-L-threonyl-L-seryl-L-glutaminyl-L-valyl-L-arginyl-L-prolyl-), AS-900004, SCH-C, ZK-811752, PD-172084, UK-427857, SB-380732, vMIP II, SB-265610, DPC-168, TAK-779 (N,N-Dimethyl-N-[4-[2-(4-methylphenyl)-6,7-dihydro-5H-benzocycloheptene-8-ylcarboxamido]benzyl]tetrahydro-2H-pyran-4-aminium chloride), TAK-220, KRH-1120, or an analogue or derivative thereof.

C. Cell Cycle Inhibitors

In one embodiment of the present invention, the pharmacologically active compound associated with the anastomotic connector device is a cell cycle inhibitor. Exemplary compounds having this biological activity include the following, where the present invention provides that each of these specifically named compounds may be placed in association with an anastomotic connection device of the present invention: taxanes (e.g., paclitaxel (discussed in more detail below) and docetaxel) (Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(40):351-386, 1993), Etanidazole, Nimorazole (B. A. Chabner and D. L. Longo. Cancer Chemotherapy and Biotherapy—Principles and Practice. Lippincott-Raven Publishers, New York, 1996, p.554), perfluorochemicals with hyperbaric oxygen, transfusion, erythropoietin, BW12C, nicotinamide, hydralazine, BSO, WR-2721, IudR, DUdR, etanidazole, WR-2721, BSO, mono-substituted keto-aldehyde compounds (L. G. Egyud. Keto-aldehyde-amine addition products and method of making same. U.S. Pat. No. 4,066,650, Jan. 3, 1978), nitroimidazole (K. C. Agrawal and M. Sakaguchi. Nitroimidazole radiosensitizers for Hypoxic tumor cells and compositions thereof. U.S. Pat. No. 4,462,992, Jul. 31, 1984), 5-substituted-4-nitroimidazoles (Adams et al., Int. J. Radiat. Biol. Relat. Stud. Phys., Chem. Med. 40(2):153-61, 1981), SR-2508 (Brown et al., Int. J. Radiat. Oncol., Biol. Phys. 7(6):695-703, 1981), 2H-isoindolediones (J. A. Myers, 2H-Isoindolediones, their synthesis and use as radiosensitizers. U.S. Pat. No. 4,494,547, Jan. 22, 1985), chiral [[(2-bromoethyl)-amino]methyl]-nitro-1H-imidazole-1-ethanol (V. G. Beylin, et al., Process for preparing chiral [[(2-bromoethyl)-amino]methyl]-nitro-1H-imidazole-1-ethanol and related compounds. U.S. Pat. No. 5,543,527, Aug. 6, 1996; U.S. Pat. No. 4,797,397; Jan. 10, 1989; U.S. Pat. No. 5,342,959, Aug. 30, 1994), nitroaniline derivatives (W. A. Denny, et al. Nitroaniline derivatives and their use as anti-tumor agents. U.S. Pat. No. 5,571,845, Nov. 5, 1996), DNA-affinic hypoxia selective cytotoxins (M. V. Papadopoulou-Rosenzweig. DNA-affinic hypoxia selective cytotoxins. U.S. Pat. No. 5,602,142, Feb. 11, 1997), halogenated DNA ligand (R. F. Martin. Halogenated DNA ligand radiosensitizers for cancer therapy. U.S. Pat. No. 5,641,764, Jun. 24, 1997), 1,2,4 benzotriazine oxides (W. W. Lee et al. 1,2,4-benzotriazine oxides as radiosensitizers and selective cytotoxic agents. U.S. Pat. No. 5,616,584, Apr. 1, 1997; U.S. Pat. No. 5,624,925, Apr. 29, 1997; Process for Preparing 1,2,4 Benzotriazine oxides. U.S. Pat. No. 5,175,287, Dec. 29, 1992), nitric oxide (J. B. Mitchell et al., Use of Nitric oxide releasing compounds as hypoxic cell radiation sensitizers. U.S. Pat. No. 5,650,442, Jul. 22, 1997), 2-nitroimidazole derivatives (M. J. Suto et al. 2-Nitroimidazole derivatives useful as radiosensitizers for hypoxic tumor cells. U.S. Pat. No. 4,797,397, Jan. 10, 1989; T. Suzuki. 2-Nitroimidazole derivative, production thereof, and radiosensitizer containing the same as active ingredient. U.S. Pat. No. 5,270,330, Dec. 14, 1993; T. Suzuki et al. 2-Nitroimidazole derivative, production thereof, and radiosensitizer containing the same as active ingredient. U.S. Pat. No. 5,270,330, Dec 14, 1993; T. Suzuki. 2-Nitroimidazole derivative, production thereof and radiosensitizer containing the same as active ingredient; Patent EP 0 513 351 B1, Jan. 24, 1991), fluorine-containing nitroazole derivatives (T. Kagiya. Fluorine-containing nitroazole derivatives and radiosensitizer comprising the same. U.S. Pat. No. 4,927,941, May 22, 1990), copper (M. J. Abrams. Copper Radiosensitizers. U.S. Pat. No. 5,100,885, Mar. 31, 1992), combination modality cancer therapy (D. H. Picker et al. Combination modality cancer therapy. U.S. Pat. No. 4,681,091, Jul. 21, 1987). 5-CldC or (d)H₄U or 5-halo-2′-halo-2′-deoxy-cytidine or -uridine derivatives (S. B. Greer. Method and Materials for sensitizing neoplastic tissue to radiation. U.S. Pat. No. 4,894,364 Jan. 16, 1990), platinum complexes (K. A. Skov. Platinum Complexes with one radiosensitizing ligand. U.S. Pat. No. 4,921,963. May 1, 1990; K. A. Skov. Platinum Complexes with one radiosensitizing ligand. Patent EP 0 287 317 A3), fluorine-containing nitroazole (T. Kagiya, et al. Fluorine-containing nitroazole derivatives and radiosensitizer comprising the same. U.S. Pat. No. 4,927,941. May 22, 1990), benzamide (W. W. Lee. Substituted Benzamide Radiosensitizers. U.S. Pat. No. 5,032,617, Jul. 16, 1991), autobiotics (L. G. Egyud. Autobiotics and their use in eliminating nonself cells in vivo. U.S. Pat. No. 5,147,652), benzamide and nicotinamide (W. W. Lee et al. Benzamide and Nictoinamide Radiosensitizers. U.S. Pat. No. 5,215,738), acridine-intercalator (M. Papadopoulou-Rosenzweig. Acridine Intercalator based hypoxia selective cytotoxins. U.S. Pat. No. 5,294,715, Mar. 15, 1994), fluorine-containing nitroimidazole (T. Kagiya et al. Fluorine containing nitroimidazole compounds. U.S. Pat. No. 5,304,654), hydroxylated texaphyrins (J. L. Sessler et al. Hydroxylated texaphrins. U.S. Pat. No. 5,457,183), hydroxylated compound derivative (T. Suzuki et al. Heterocyclic compound derivative, production thereof and radiosensitizer and antiviral agent containing said derivative as active ingredient. Publication Number 011106775 A (Japan); T. Suzuki et al. Heterocyclic compound derivative, production thereof and radiosensitizer, antiviral agent and anti cancer agent containing said derivative as active ingredient. Publication Number 01139596A (Japan), Nov. 25, 1987; S. Sakaguchi et al. Heterocyclic compound derivative, its production and radiosensitizer containing said derivative as active ingredient; Publication Number 63170375 A (Japan)), fluorine containing 3-nitro-1,2,4-triazole (T. Kagitani et al. Novel fluorine-containing 3-nitro-1,2,4-triazole and radiosensitizer containing same compound. Publication Number 02076861 A (Japan)), 5-thiotretrazole derivative or its salt (E. Kano et al. Radiosensitizer for Hypoxic cell. Publication Number 61010511 A (Japan)), Nitrothiazole (T. Kagitani et al. Radiation-sensitizing agent. Publication Number 61167616 A (Japan)), imidazole derivatives (S. Inayma et al. Imidazole derivative. Publication Number 6203767 A (Japan); Publication Number 62030768 A (Japan); Publication Number 62030777 A (Japan)), 4-nitro-1,2,3-triazole (T. Kagitani et al. Radiosensitizer. Publication Number 62039525 A (Japan)), 3-nitro-1,2,4-triazole (T. Kagitani et al. Radiosensitizer. Publication Number 62138427 A (Japan)), Carcinostatic action regulator (H. Amagase. Carcinostatic action regulator. Publication Number 63099017 A (Japan)), 4,5-dinitroimidazole derivative (S. Inayama. 4,5-Dinitroimidazole derivative. Publication Number 63310873 A (Japan)), nitrotriazole Compound (T. Kagitanil Nitrotriazole Compound. Publication Number 07149737 A (Japan)), cisplatin, doxorubin, misonidazole, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide (I. F. Tannock. Review Article: Treatment of Cancer with Radiation and Drugs. Journal of Clinical Oncology 14(12):3156-3174, 1996), camptothecin (Ewend M. G. et al. Local delivery of chemotherapy and concurrent external beam radiotherapy prolongs survival in metastatic brain tumor models. Cancer Research 56(22):5217-5223, 1996) and paclitaxel (Tishler R. B. et al. Taxol: a novel radiation sensitizer. International Journal of Radiation Oncology and Biological Physics 22(3):613-617, 1992).

A number of the above-mentioned cell cycle inhibitors also have a wide variety of analogues and derivatives, where in one aspect the present invention provides that analogues and derivatives of each of the afore-mentioned compounds may be associated with an anastomotic connection device. Exemplary analogues and derivatives include, without limitation: cisplatin, cyclophosphamide, misonidazole, tiripazamine, nitrosourea, mercaptopurine, methotrexate, fluorouracil, epirubicin, doxorubicin, vindesine and etoposide. Analogues and derivatives include (CPA)₂Pt[DOLYM] and (DACH)Pt[DOLYM] cisplatin (Choi et al., Arch. Pharmacal Res. 22(2):151-156, 1999), Cis-[PtCl₂(4,7-H-5-methyl-7-oxo]1,2,4[triazolo[1,5-a]pyrimidine)₂] (Navarro et al., J. Med. Chem. 41(3):332-338, 1998), [Pt(cis-1,4-DACH)(trans-Cl₂)(CBDCA)] . ½MeOH cisplatin (Shamsuddin et al., Inorg. Chem. 36(25):5969-5971, 1997), 4-pyridoxate diamine hydroxy platinum (Tokunaga et al., Pharm. Sci. 3(7):353-356, 1997), Pt(II) . . . Pt(II) (Pt₂[NHCHN(C(CH₂)(CH₃))]₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer& Bednarski, J. Inorg. Biochem. 62(4):281-298, 1996), trans,cis-[Pt(OAc)₂I₂(en)] (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-[Pt(NH₃)(4-aminoTEMP-O){d(GpG)}] (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), Cl-973 cisplatin analogue (Yang et al., Int. J. Oncol. 5(3):597-602, 1994), cis-diaminedichloroplatinum(II) and its analogues cis-1,1-cyclobutanedicarbosylato(2R)-2-methyl-1,4-butanediammineplatinum(II) and cis-diamine(glycolato)platinum (Claycamp & Zimbrick, J. Inorg. Biochem., 26(4):257-67, 1986; Fan et al., Cancer Res. 48(11):3135-9, 1988; Heiger-Bernays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol. 33(1):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diaminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum (II) and 3H-cis-1,2-diaminocyclohexanemalonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diamine(1,1-cyclobutanedicarboxylato-)platinum(lI) (carboplatin, JM8) and ethylenediamminemalonatoplatinum(II) (JM40) (Begg et al., Radiother. Oncol. 9(2):157-65, 1987), JM8 and JM9 cisplatin analogues (Harstrick et al., Int. J. Androl. 10(1); 139-45, 1987), (NPr4)2((PtCL4).cis-(PtCI2-(NH2Me)2)) (Brammer et al., J. Chem. Soc., Chem. Commun. 6:443-5, 1987), aliphatic tricarboxylic acid platinum complexes (EPA 185225), cis-dichloro(amino acid)(tert-butylamine)platinum(II) complexes (Pasini & Bersanetti, Inorg. Chim. Acta 107(4):259-67, 1985); 4-hydroperoxycylcophosphamide (Ballard et al., Cancer Chemother. Pharmacol. 26(6):397-402, 1990), acyclouridine cyclophosphamide derivatives (Zakerinia et al., Helv. Chim. Acta 73(4):912-15, 1990), 1,3,2-dioxa- and -oxazaphosphorinane cyclophosphamide analogues (Yang et al., Tetrahedron 44(20):6305-14, 1988), C5-substituted cyclophosphamide analogues (Spada, University of Rhode Island Dissertation, 1987), tetrahydrooxazine cyclophosphamide analogues (Valente, University of Rochester Dissertation, 1988), phenyl ketone cyclophosphamide analogues (Hales et al., Teratology 39(1):31-7, 1989), phenylketophosphamide cyclophosphamide analogues (Ludeman et al., J. Med. Chem. 29(5):716-27, 1986), ASTA Z-7557 cyclophosphamide analogues (Evans et al., Int. J. Cancer 34(6):883-90, 1984), 3-(1-oxy-2,2,6,6-tetramethyl-4-piperidinyl)cyclophosphamide (Tsui et al., J. Med. Chem. 25(9):1106-10, 1982), 2-oxobis(2-β-chloroethylamino)-4-,6-dimethyl-1,3,2-oxazaphosphorinane cyclophosphamide (Carpenter et al., Phosphorus Sulfur 12(3):287-93, 1982), 5-fluoro- and 5-chlorocyclophosphamide (Foster et al., J. Med. Chem. 24(12):1399-403, 1981), cis- and trans-4-phenylcyclophosphamide (Boyd et al., J. Med. Chem. 23(4):372-5, 1980), 5-bromocyclophosphamide, 3,5-dehydrocyclophosphamide (Ludeman et al., J. Med. Chem. 22(2):151-8, 1979), 4-ethoxycarbonyl cyclophosphamide analogues (Foster, J. Pharm. Sci. 67(5):709-10, 1978), arylaminotetrahydro-2H-1,3,2-oxazaphosphorine 2-oxide cyclophosphamide analogues (Hamacher, Arch. Pharm. (Weinheim, Ger.) 310(5):J,428-34, 1977), NSC-26271 cyclophosphamide analogues (Montgomery & Struck, Cancer Treat. Rep. 60(4):J381-93, 1976), benzo annulated cyclophosphamide analogues (Ludeman & Zon, J. Med. Chem. 18(12):J1251-3, 1975), 6-trifluoromethylcyclophosphamide (Farmer & Cox, J. Med. Chem. 18(11):J1106-10, 1975), 4-methylcyclophosphamide and 6-methycyclophosphamide analogues (Cox et al., Biochem. Pharmacol. 24(5):J599-606, 1975); FCE 23762 doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Nat'l Cancer Inst. 89(16):1217-1223, 1997), 4-demethoxy-7-O-[2,6-dideoxy-4-O-(2,3,6-trideoxy-3-amino-□-L-lyxo-hexopyranosyl )-α-L-lyxo-hexopyranosyl]-adriamicinone doxorubicin disaccharide analog (Monteagudo et al., Carbohydr. Res. 300(1):11-16, 1997), 2-pyrrolinodoxorubicin (Nagy et al., Proc. Nat'l Acad. Sci. U.S.A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int. J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Willner et al., Bioconjugate Chem. 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14):3682-9, 1991), 4-demethoxy-3′-N-trifluoroacetyidoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ., 16(Biol. 1):21-7, 1988), 4′-deoxydoxorubicin (Schoelzel et al., Leuk. Res. 10(12):1455-9, 1986), 4-demethyoxy-4′-o-methyidoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20,1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277); 4,5-dimethylmisonidazole (Born et al., Biochem. Pharmacol. 43(6):1337-44, 1992), azo and azoxy misonidazole derivatives (Gattavecchia & Tonelli, Int. J. Radiat Biol. Relat. Stud. Phys., Chem. Med. 45(5):469-77, 1984); RB90740 (Wardman et al., Br. J. Cancer, 74 Suppl. (27):S70-S74,1996); 6-bromo and 6-chloro-2,3-dihydro-1,4-benzothiazines nitrosourea derivatives (Rai et al., Heterocycl. Commun. 2(6):587-592, 1996), diamino acid nitrosourea derivatives (Dulude et al., Bioorg. Med. Chem. Lett. 4(22):2697-700, 1994; Dulude et al., Bioorg. Med. Chem. 3(2):151-60, 1995), amino acid nitrosourea derivatives (Zheleva et al., Pharmazie 50(1):25-6, 1995), 3′,4′-didemethoxy-3′,4′-dioxo-4-deoxypodophyllotoxin nitrosourea derivatives (Miyahara et al., Heterocycles 39(1):361-9, 1994), ACNU (Matsunaga et al., Immunopharmacology 23(3):199-204, 1992), tertiary phosphine oxide nitrosourea derivatives (Guguva et al., Pharmazie 46(8):603, 1991), sulfamerizine and sulfamethizole nitrosourea derivatives (Chiang et al., Zhonghua Yaozue Zazhi 43(5):401-6, 1991), thymidine nitrosourea analogues (Zhang et al., Cancer Commun. 3(4):119-26, 1991), 1,3-bis(2-chloroethyl)-1-nitrosourea (August et al., Cancer Res. 51(6):1586-90, 1991), 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine (Boutin et al., Eur. J. Cancer Clin. Oncol. 25(9):1311-16, 1989), pyrimidine (II) nitrosourea derivatives (Wei et al., Chung-hua Yao Hsueh Tsa Chih 41(1):19-26, 1989), CGP 6809 (Schieweck et al., Cancer Chemother. Pharmacol. 23(6):341-7, 1989), B-3839 (Prajda et al., In Vivo 2(2):151-4, 1988), 5-halogenocytosine nitrosourea derivatives (Chiang & Tseng, T'ai-wan Yao Hsueh Tsa Chih 38(1):37-43, 1986), 1-(2-chloroethyl)-3-isobutyl-3-(β-maltosyl)-1-nitrosourea (Fujimoto & Ogawa, J. Pharmacobio-Dyn. 10(7):341-5, 1987), sulfur-containing nitrosoureas (Tang et al., Yaoxue Xuebao 21(7):502-9, 1986), sucrose, 6-((((2-chloroethyl)nitrosoamino-)carbonyl)amino)-6-deoxysucrose (NS-1C) and 6′-((((2-chloroethyl) nitrosoamino)carbonyl)amino)-6′-deoxysucrose (NS-1D) nitrosourea derivatives (Tanoh et al., Chemotherapy (Tokyo) 33(11):969-77, 1985), CNCC, RFCNU and chlorozotocin (Mena et al., Chemotherapy (Basel) 32(2):131-7, 1986), CNUA (Edanami et al., Chemotherapy (Tokyo) 33(5):455-61, 1985), 1-(2-chloroethyl)-3-isobutyl-3-(β-maltosyl)-1-nitrosourea (Fujimoto & Ogawa, Jpn. J. Cancer Res. (Gann) 76(7):651-6, 1985), choline-like nitrosoalkylureas (Belyaev et al., Izv. Akad. NAUK SSSR, Ser. Khim. 3:553-7, 1985), sucrose nitrosourea derivatives (JP 84219300), sulfa drug nitrosourea analogues (Chiang et al., Proc. Nat'l Sci. Counc., Repub. China, Part A 8(1 ):18-22, 1984), DONU (Asanuma et al., J. Jpn. Soc. Cancer Ther. 17(8):2035-43, 1982), N,N′-bis (N-(2-chloroethyl)-N-nitrosocarbamoyl)cystamine (CNCC) (Blazsek et al., Toxicol. Appl. Pharmacol. 74(2):250-7, 1984), dimethylnitrosourea (Krutova et al., Izv. Akad. NAUK SSSR, Ser. Biol. 3:439-45, 1984), GANU (Sava & Giraldi, Cancer Chemother. Pharmacol. 10(3):167-9, 1983), CCNU (Capelli et al., Med., Biol., Environ. 11(1):111-16, 1983), 5-aminomethyl-2′-deoxyuridine nitrosourea analogues (Shiau, Shih Ta Hsueh Pao (Taipei) 27:681-9, 1982), TA-077 (Fujimoto & Ogawa, Cancer Chemother. Pharmacol. 9(3):134-9, 1982), gentianose nitrosourea derivatives (JP 82 80396), CNCC, RFCNU, RPCNU AND chlorozotocin (CZT) (Marzin et al., INSERM Symp., 19(Nitrosoureas Cancer Treat.):165-74, 1981), thiocolchicine nitrosourea analogues (George, Shih Ta Hsueh Pao (Taipei) 25:355-62, 1980), 2-chloroethylnitrosourea (Zeller & Eisenbrand, Oncology 38(1):39-42, 1981), ACNU, (1-(4-amino-2-methyl-5-pyrimid inyl)methyl-3-(2-chloroethyl)-3-nitrosourea hydrochloride) (Shibuya et al., Gan To Kagaku Ryoho 7(8):1393-401, 1980), N-deacetylmethyl thiocolchicine nitrosourea analogues (Lin et al., J. Med. Chem. 23(12):1440-2, 1980), pyridine and piperidine nitrosourea derivatives (Crider et al., J. Med. Chem. 23(8):848-51, 1980), methyl-CCNU (Zimber & Perk, Refu. Vet. 35(1):28, 1978), phensuzimide nitrosourea derivatives (Crider et al., J. Med. Chem. 23(3):324-6, 1980), ergoline nitrosourea derivatives (Crider et al., J. Med. Chem. 22(1):32-5, 1979), glucopyranose nitrosourea derivatives (JP 78 95917),1-(2-chloroethyl)-3-cyclohexyl-1-nitrosourea (Farmer et al., J. Med. Chem. 21(6):514-20, 1978), 4-(3-(2-chloroethyl)-3-nitrosoureid-o)-cis-cyclohexanecarboxylic acid (Drewinko et al., Cancer Treat. Rep. 61(8):J1513-18, 1977), RPCNU (ICIG 1163) (Larnicol et al., Biomedicine 26(3):J176-81, 1977), IOB-252 (Sorodoc et al., Rev. Roum. Med., Virol. 28(1):J 55-61, 1977), 1,3-bis(2-chloroethyl)-1-nitrosourea (BCNU) (Siebert & Eisenbrand, Mutat. Res. 42(1):J45-50, 1977), 1-tetrahydroxycyclopentyl-3-nitroso-3-(2-chloroethyl )-urea (U.S. Pat. No. 4,039,578), d-1-1-(β-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7, 1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7, 1981); indoline ring and a modified ornithine or glutamic acid-bearing methotrexate derivatives (Matsuoka et al., Chem. Pharm. Bull. 45(7):1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaaminopterin analogues (DeGraw et al., J. Med. Chem. 40(3):370-376, 1997), 5-deazaaminopterin and 5,10-dideazaaminopterin methotrexate analogues (Piper et al., J. Med. Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm., Biopharm. Pharm. Technol., 563-4, 1995), L-threo-(2S, 4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8, 1995), N-(□-aminoacyl) methotrexate derivatives (Cheung et al., Pteridines 3(1-2):101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(12):2400-3, 1991), βγ-methano methotrexate analogues (Rosowsky et al., Pteridines 2(3):133-9, 1991), 10-deazaaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl) methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), □-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), □- and □-substituted methotrexate analogues (Tsushima et al., Tetrahedron 44(17):5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed. Polym.):311-24, 1987), methotrexate-γ-dimyristoylphophatidylethanolamine (Kinsky et al., Biochim. Biophys. Acta 917(2):211-18, 1987), methotrexate polyglutamate analogues (Rosowsky et al., Chem. Biol. Pteridines, Pteridines Folid Acid Deriv., Proc. Int. Symp. Pteridines Folid Acid Deriv.: Chem., Biol. Clin. Aspects: 985-8, 1986), poly-γ-glutamyl methotrexate derivatives (Kisliuk et al., Chem. Biol. Pteridines, Pteridines Folid Acid Deriv., Proc. Int. Symp. Pteridines Folid Acid Deriv.: Chem., Biol. Clin. Aspects: 989-92, 1986), deoxyuridylate methotrexate derivatives (Webber et al., Chem. Biol. Pteridines, Pteridines Folid Acid Deriv., Proc. Int. Symp. Pteridines Folid Acid Deriv.: Chem., Biol. Clin. Aspects: 659-62, 1986), iodoacetyl lysine methotrexate analogue (Delcamp et al., Chem. Biol. Pteridines, Pteridines Folid Acid Deriv., Proc. Int. Symp. Pteridines Folid Acid Deriv.: Chem., Biol. Clin. Aspects: 807-9, 1986), 2,.omega.-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122 (Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (U.S. Pat. No. 4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.—Chim. Ther. 19(3):267-73, 1984), poly (L-lysine) methotrexate conjugates (Rosowsky et al., J. Med. Chem. 27(7):888-93, 1984), dilysine and trilysine methotrexate derivates (Forsch & Rosowsky, J. Org. Chem. 49(7):1305-9, 1984), 7-hydroxymethotrexate (Fabre et al., Cancer Res. 43(10):4648-52, 1983), poly-□-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol. 17(1):105-10, 1980), halogenated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220); N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9, 1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology45(3):144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-actyl-3-O-toluyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin (Lanius, Adv. Chemother. Gastrointest. Cancer, (Int. Symp.), 159-67, 1984); N-substituted deacetylvinblastine amide (vindesine) sulfates (Conrad et al., J. Med. Chem. 22(4):391-400, 1979); and Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989). In separate aspects, the present invention provides that each of the aforementioned cell cycle inhibitors is placed in association with an anastomotic connector device in a therapeutically effective manner and at a therapeutically effective concentration.

Within one preferred embodiment of the invention, the cell cycle inhibitor is paclitaxel, a compound which disrupts mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216, 1993). “Paclitaxel” (which should be understood herein to include formulations, prodrugs, analogues and derivatives such as, for example, TAXOL®, TAXOTERE®, docetaxel, 10-desacetyl analogues of paclitaxel and 3′N-desbenzoyl-3′N-t-butoxy carbonyl analogues of paclitaxel) may be readily prepared utilizing techniques known to those skilled in the art (see, e.g., Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J.Am. Chem. Soc. 110:6558-6560, 1988), or obtained from a variety of commercial sources, including for example, Sigma Chemical Co., St. Louis, Mo. (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′- and/or 7-O-ester derivatives), (2′- and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2'succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-( N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl )taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin IlIl analogs bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the Cell Cycle Inhibitor is a taxane having the formula (C1):

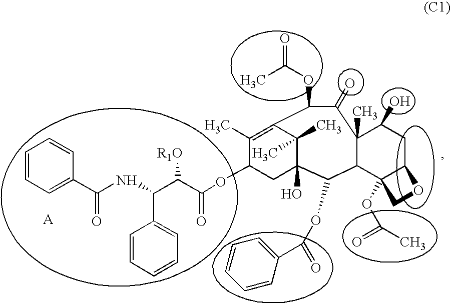

where the gray-highlighted portions may be substituted and the non-highlighted portion is the taxane core. A side-chain (labeled “A” in the diagram ) is desirably present in order for the compound to have good activity as a Cell Cycle Inhibitor. Examples of compounds having this structure include paclitaxel (Merck Index entry 7117), docetaxol (Taxotere, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogs and derivatives are disclosed in U.S. Pat. No. 5,440,056 as having the structure (C2):

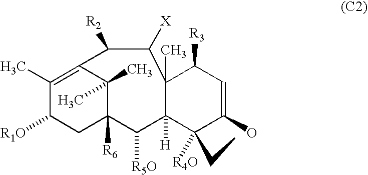

wherein X may be oxygen (paclitaxel), hydrogen (9-deoxy derivatives), thioacyl, or dihydroxyl precursors; R₁ is selected from paclitaxel or taxotere side chains or alkanoyl of the formula (C3)

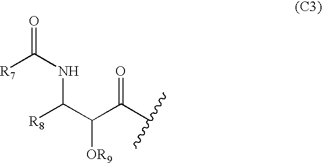

wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogs and derivatives useful as Cell Cycle Inhibitors in the present invention are disclosed in PCT International Patent Application No. WO 93/10076. As disclosed in this publication, the analog or derivative should have a side chain attached to the taxane nucleus at C₁₃, as shown in the structure below (formula C4), in order to confer antitumor activity to the taxane.

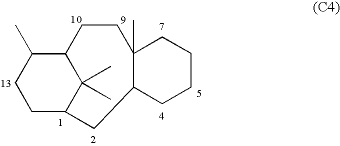

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, the taxane-based Cell Cycle Inhibitor useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Taxanes in general, and paclitaxel is particular, is considered to function as a Cell Cycle Inhibitor by acting as a anti-microtubule agent, and more specifically as a stabilizer. These compounds have been shown useful in the treatment of proliferative disorders, including: non-small cell (NSC) lung; small cell lung; breast; prostate; cervical; endometrial; head and neck cancers.

The agent associated with an anastomotic connector device may, in one aspect of the invention, have anti-microtubule activity, where assays to test for anti-microtubule activity are well known in the art and include, for example, Allan, V. J. and Vale, R. D. 1991. Cell cycle control of microtubule-based transport and tubule formation in vitro. J. Cell Biol. 113:347-359; Coue, M., Lombillo, V. A., and McIntosh, J. R. 1991. Microtubule depolymerization promotes particle and chromosome movement in vitro. J. Cell Biol. 112:1165-1175; and Dabora, S. L. and Sheetz, M. P. 1988. Microtubule dependent formation of a tubular vesicular network with characteristics of the endoplasmic reticulum from cultured cell extracts. Cell 54:27-35.

In another aspect, the Cell Cycle Inhibitor that is associated with an anastomotic connection device according to the present invention is a Vinca Alkaloid. Vinca alkaloids have the following general structure. They are indole-dihydroindole dimers.

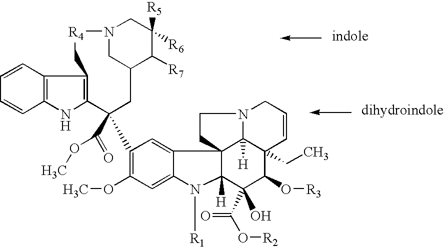

As disclosed in U.S. Pat. Nos. 4,841,045 and 5,030,620, R₁ can be a formyl or methyl group or alternately H. R₁ could also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)—R where R is NH₂, an amino acid ester or a peptide ester. R₃ is typically C(O)CH₃, CH₃ or H. Alternately a protein fragment may be linked by a bifunctional group such as maleoyl amino acid. R₃ could also be substituted to form an alkyl ester which may be further substituted. R₄ may be —CH₂— or a single bond. R₅ and R₆ may be either H, OH or a lower alkyl, typically —CH₂CH₃. Alternatively R₆ and R₇ may together form an oxetane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary Vinca Alkaloids are vinblastine, vincristine, vincristine sulfate, vindesine, and vinorelbine, having the structures:

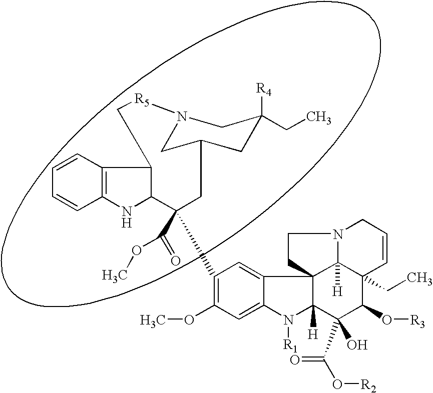

| | R₁ | R₂ | R₃ | R₄ | R₅ |
|---|---|---|---|---|---|
| Vinblastine: | CH₃ | CH₃ | C(O)CH₃ | OH | CH₂ |
| Vincristine: | CH₂O | CH₃ | C(O)CH₃ | OH | CH₂ |
| Vindesine: | CH₃ | NH₂ | H | OH | CH₂ |
| Vinorelbine: | CH₃ | CH₃ | CH₃ | H | single bond |

Analogs typically require the side group (shaded area) in order to have activity. These compounds are thought to act as Cell Cycle Inhibitors by functioning as anti-microtubule agents, and more specifically to inhibit polymerization. These compounds have been shown useful in treating proliferative disorders, including NSC lung; small cell lung; breast; prostate; brain; head and neck; retinoblastoma; bladder; and penile cancers; and soft tissue sarcoma.

In another aspect, the Cell Cycle Inhibitor that is associated with an anastomotic connection device according to the present invention is Camptothecin, or an analog or derivative thereof. Camptothecins have the following general structure.

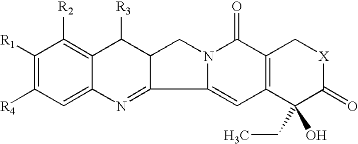

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxy camptothecin, SN-38, 9-nitrocamptothecin, 10-hydroxycamptothecin. Exemplary compounds have the structures:

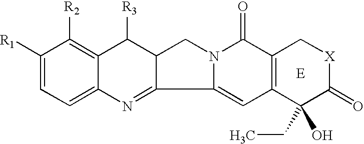

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity. These compounds are useful to as Cell Cycle Inhibitors, where they function as Topoisomerase I Inhibitors and/or DNA cleavage agents. Topoisomerase I Inhibitors may be identified using a relaxation assay such as is described by Liu, L. F. and Miller, K. G. (1981) PNAS 76: 3487-3491. They have been shown useful in the treatment of proliferative disorders, including, for example, NSC lung; small cell lung; and cervical cancers.

In another aspect, the Cell Cycle Inhibitor that is associated with an anastomotic connection device according to the present invention is a Podophyllotoxin, or a derivative or an analog thereof. Exemplary compounds of this type are Etoposide or Teniposide, which have the following structures:

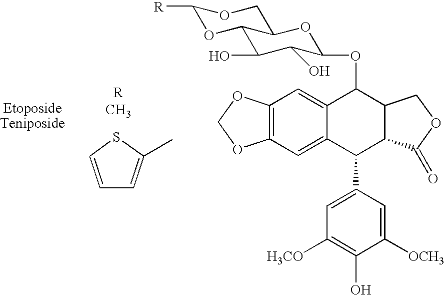

These compounds are thought to function as Cell Cycle Inhibitors by being Topoisomerase II Inhibitors and/or by DNA cleaving agents. Topoisomerase II Inhibitors may be identified using an activity assay such as is described by Liu, L. F. et al. (1981) Nucleic Acids Res. 9: 3979-3989. They have been shown useful as antiproliferative agents in, e.g., small cell lung, prostate, and brain cancers, and in retinoblastoma.

In another aspect, the Cell Cycle Inhibitor that is associated with an anastomotic connection device according to the present invention is an Anthracycline. Anthracyclines have the following general structure, where the R groups may be a variety of organic groups:

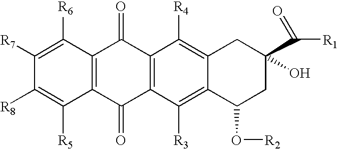

According to U.S. Pat. No. 5,594,158, suitable R groups are: R₁ is CH₃ or CH₂OH; R₂ is daunosamine or H; R₃ and R₄ are independently one of OH, NO₂, NH₂, F, Cl, Br, I, CN, H or groups derived from these; R₅₋₇ are all H or R₅ and R₆ are H and R₇ and R₈ are alkyl or halogen, or vice versa: R₇ and R₈ are H and R₅ and R₆ are alkyl or halogen.

According to U.S. Pat. No. 5,843,903, R₂ may be a conjugated peptide. According to U.S. Pat. Nos. 4,215,062 and 4,296,105, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure:

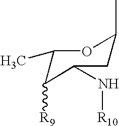

in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxy, mercapto, phenyl, benzyl or methylthio (see U.S. Pat. No. 4,296,105).

Exemplary Anthracycline are Doxorubicin, Daunorubicin, Idarubicin, Epirubicin, Pirarubicin, Zorubicin, and Carubicin. Suitable compounds have the structures:

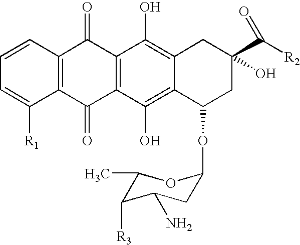

| | R₁ | R₂ | R₃ |
|---|---|---|---|
| Doxorubicin: | OCH₃ | CH₂OH | OH Out of |
| | | | ring plane |
| Epirubion: | OCH₃ | CH₂OH | OH in ring |
| (4′ epimer of doxorubicin) | | | plane |
| Daunorubicin: | OCH₃ | CH₃ | OH Out of |
| | | | ring plane |
| Idarubicin: | H | CH₃ | OH out of |
| | | | ring plane |
| Pirarubicin | OCH₃ | OH | A |
| Zorubicin | OCH₃ | ═N—NHC(O)C₆H₅ | B |
| Carubicin | OH | CH₃ | B |

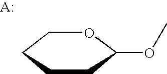

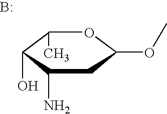

Other suitable Anthracyclines are Anthramycin, Mitoxantrone, Menogaril, Nogalamycin, Aclacinomycin A, Olivomycin A, Chromomycin A₃, and Plicamycin having the structures:

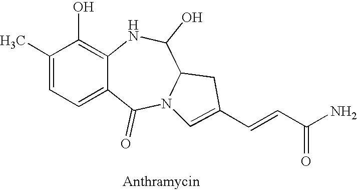

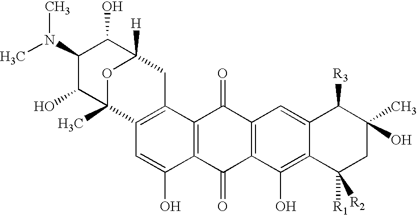

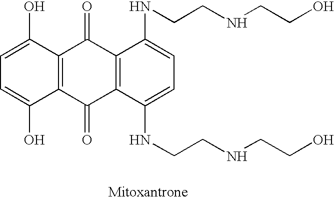

| | R₁ | R₂ | R₃ |
|---|---|---|---|
| Menogaril | H | OCH₃ | H |
| Nogalamycin | O-sugar | H | COOCH₃ |

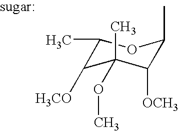

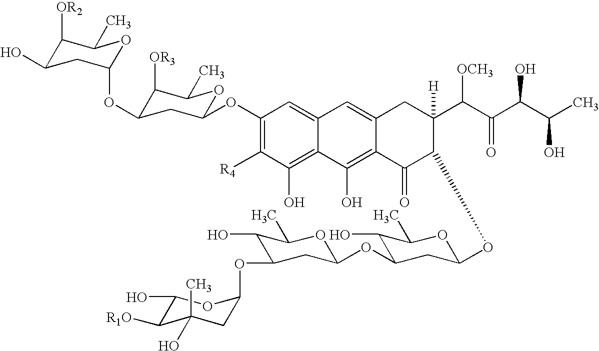

| | R₁ | R₂ | R₃ | R₄ |
|---|---|---|---|---|
| Olivomycin A | COCH(CH₃)₂ | CH₃ | COCH₃ | H |
| Chromomycin A₃ | COCH₃ | CH₃ | COCH₃ | CH₃ |
| Plicamycin | H | H | H | CH₃ |

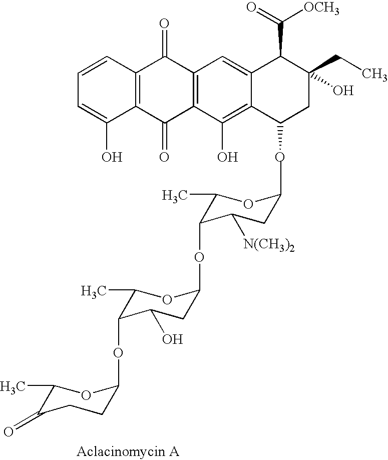

These compounds are thought to function as Cell Cycle Inhibitors by being Topoisomerase Inhibitors and/or by DNA cleaving agents. They have been shown useful in the treatment of proliferative disorders, including small cell lung; breast; endometrial; head and neck; retinoblastoma; liver; bile duct; islet cell; and bladder cancers; and soft tissue sarcoma.

In another aspect, the Cell Cycle Inhibitor that is associated with an anastomotic connection device according to the present invention is a Platinum compound. In general, suitable platinum complexes may be of Pt(II) or Pt(IV) and have this basic structure:

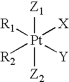

wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

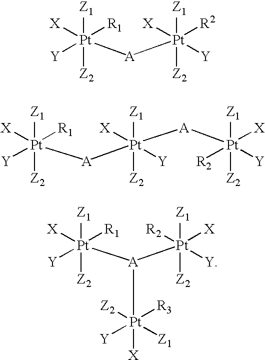

Exemplary Platinum compound are Cisplatin, Carboplatin, Oxaliplatin, and Miboplatin having the structures:

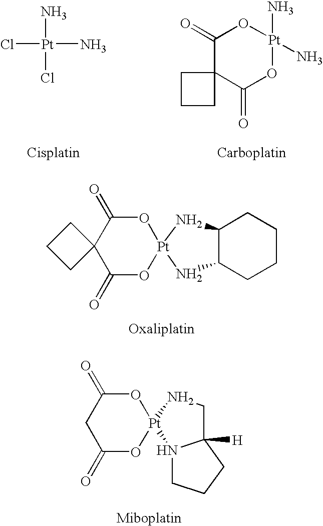

These compounds are thought to function as Cell Cycle Inhibitors by binding to DNA, i.e., acting as alkylating agents of DNA. These compounds have been shown useful in the treatment of cell proliferative disorders, including, e.g., NSC lung; small cell lung; breast; cervical; brain; head and neck; esophageal; retinoblastom; liver; bile duct; bladder; penile; and vulvar cancers; and soft tissue sarcoma.

In another aspect, the Cell Cycle Inhibitor that is associated with an anastomotic connection device according to the present invention is a Nitrosourea. Nitrosourease have the following general structure (C5), where typical R groups are shown below.

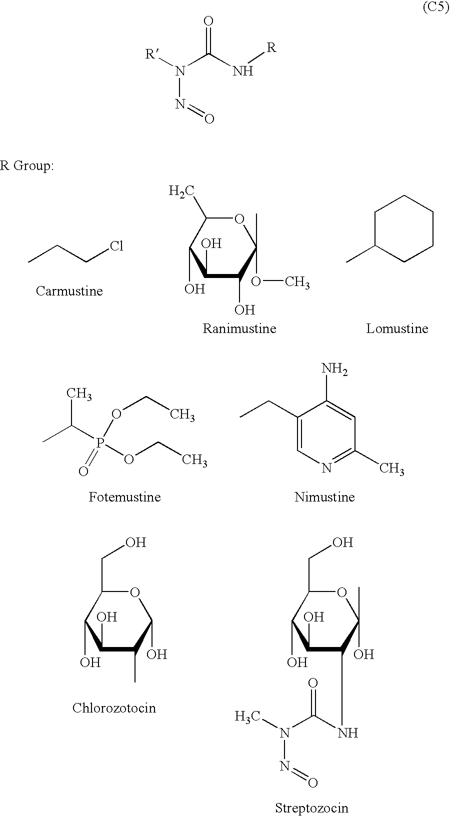

Other suitable R groups include cyclic alkanes, alkanes, halogen substituted groups, sugars, aryl and heteroaryl groups, phosphonyl and sulfonyl groups. As disclosed in U.S. Pat. No. 4,367,239, R may suitably be CH₂—C(X)(Y)(Z), wherein X and Y may be the same or different members of the following groups: phenyl, cyclyhexyl, or a phenyl or cyclohexyl group substituted with groups such as halogen, lower alkyl (C₁₋₄), trifluoro methyl, cyano, phenyl, cyclohexyl, lower alkyloxy (C₁₋₄). Z has the following structure: -alkylene-N—R₁R₂, where R₁ and R₂ may be the same or different members of the following group: lower alkyl (C₁₋₄) and benzyl, or together R₁ and R₂ may form a saturated 5 or 6 membered heterocyclic such as pyrrolidine, piperidine, morfoline, thiomorfoline, N-lower alkyl piperazine, where the heterocyclic may be optionally substituted with lower alkyl groups.

As disclosed in U.S. Pat. No. 6,096,923, R and R′ of formula (C5) may be the same or different, where each may be a substituted or unsubstituted hydrocarbon having 1-10 carbons. Substitutions may include hydrocarbyl, halo, ester, amide, carboxylic acid, ether, thioether and alcohol groups. As disclosed in U.S. Pat. No. 4,472,379, R of formula (C5) may be an amide bond and a pyranose structure (e.g., Methyl 2′-[N-[N-(2-chloroethyl)-N-nitroso-carbamoyl]-glycyl]amino-2′-deoxy-α-D-glucopyranoside). As disclosed in U.S. Pat. No. 4,150,146, R of formula (C5) may be an alkyl group of 2 to 6 carbons and may be substituted with an ester, sulfonyl, or hydroxyl group. It may also be substituted with a carboxylica acid or CONH₂ group.

Exemplary Nitrosourea are BCNU (Carmustine), Methyl-CCNU (Semustine), CCNU (Lomustine), Ranimustine, Nimustine, Chlorozotocin, Fotemustine, Streptozocin, and Streptozocin, having the structures:

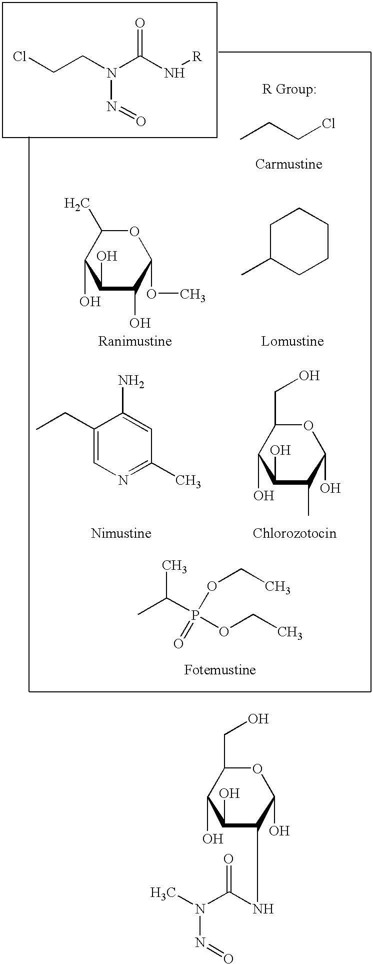

These nitrosourea compounds are thought to function as Cell Cycle Inhibitor by binding to DNA, that is, by functioning as DNA alkylating agents. These Cell Cycle Inhibitors have been shown useful in treating cell proliferative disorders such as, for example, islet cell; small cell lung; melanoma; and brain cancers.

In another aspect, the Cell Cycle Inhibitor that is associated with an anastomotic connection device according to the present invention is a Nitroimidazole, where exemplary Nitroimidazoles are Metronidazole, Benznidazole, Etanidazole, and Misonidazole, having the structures:

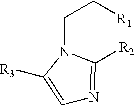

| | R₁ | R₂ | R₃ |
|---|---|---|---|
| Metronidazole | OH | CH₃ | NO₂ |
| Benznidazole | C(O)NHCH₂-benzyl | NO₂ | H |
| Etanidazole | CONHCH₂CH₂ | NO₂ | H |

Suitable nitroimidazole compounds are disclosed in, e.g., U.S. Pat. Nos. 4,371,540 and 4,462,992.

In another aspect, the Cell Cycle Inhibitor that is associated with an anastomotic connection device according to the present invention is a Folic acid antagonist, such as Methotrexate or derivatives or analogs thereof, including Edatrexate, Trimetrexate, Raltitrexed, Piritrexim, Denopterin, Tomudex, and Pteropterin. Methotrexate analogs have the following general structure:

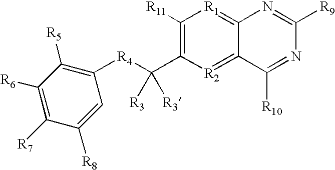

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure:

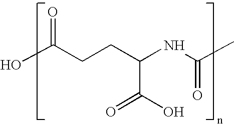

wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

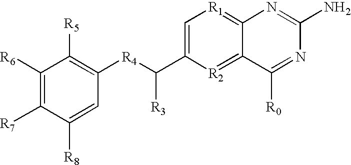

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A(n = 1) | H |
| Edatrexate | NH₂ | N | N | H | N(CH₃CH₃) | H | H | A(n = 1) | H |
| Trimetrexate | NH₂ | N | C(CH₃) | H | NH | H | OCH₃ | OCH₃ | OCH₃ |
| Ptaropterin | NH₂ | N | N | H | N(CH₃) | H | H | A(n = 3) | H |
| Denopterin | OH | N | N | CH₃ | N(CH₃) | H | H | A(n = 1) | H |
| Piritrexim | NH₂ | N | C(CH₃)H | single | OCH₃ | H | H | OCH₃ | H |
| | | | | bond | | | | | |

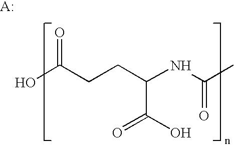

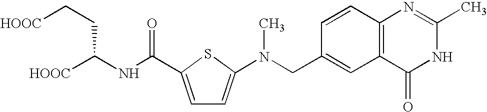

These compounds are thought to function as Cell Cycle Inhibitors by serving as antimetabolites of folic acid. They have been shown useful in the treatment of cell proliferative disorders including, for example, soft tissue sarcoma, small cell lung, breast, brain, head and neck, bladder, and penile cancers.

In another aspect, the Cell Cycle Inhibitor that is associated with an anastomotic connection device according to the present invention is a Cytidine Analog, such as Cytarabine or derivatives or analogs thereof, including Enocitabine, FMdC ((E(-2′-deoxy-2′-(fluoromethylene)cytid ine), Gemcitabine, 5-Azacitidine, Ancitabine, and 6-Azauridine. Exemplary compounds have the structures:

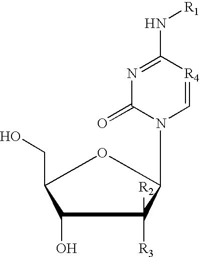

| | R₁ | R₂ | R₃ | R₄ |
|---|---|---|---|---|
| Cytarabine | H | OH | H | CH |
| Enocitabine | C(O)(CH₂)₂₀CH₃ | OH | H | CH |
| Gemcitabine | H | F | F | CH |
| Azacitidine | H | H | OH | N |
| FMdC | H | CH₂F | H | CH |

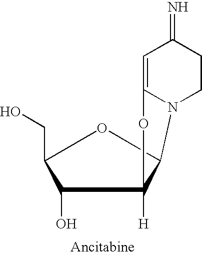

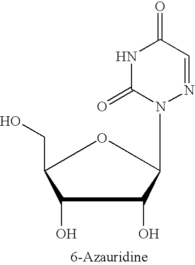

These compounds are thought to function as Cell Cycle Inhibitors as acting as antimetabolites of pyrimidine. These compounds have been shown useful in the treatment of cell proliferative disorders including, for example, pancreatic, breast, cervical, NSC lung, and bile duct cancers.

In another aspect, the Cell Cycle Inhibitor that is associated with an anastomotic connection device according to the present invention is a Pyrimidine analog. In one aspect, the Pyrimidine analogs have the general structure:

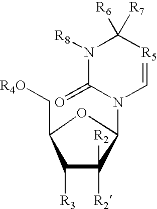

wherein positions 2′,3′and 5′ on the sugar ring (R₂, R₃ and R₄, respectively) can be H, hydroxyl, phosphoryl (see, e.g., U.S. Pat. No. 4,086,417) or ester (see, e.g., U.S. Pat. No. 3,894,000). Esters can be of alkyl, cycloalkyl, aryl or heterocyclo/aryl types. The 2′ carbon can be hydroxylated at either R₂ or R₂′, the other group is H. Alternately, the 2′ carbon can be substituted with halogens e.g., fluoro or difluoro cytidines such as Gemcytabine. Alternately, the sugar can be substituted for another heterocyclic group such as a furyl group or for an alkane, an alkyl ether or an amide linked alkane such as C(O)NH(CH₂)₅CH₃. The 2° amine can be substituted with an aliphatic acyl (R₁) linked with an amide (see, e.g., U.S. Pat. No. 3,991,045) or urethane (see, e.g., U.S. Pat. No. 3,894,000) bond. It can also be further substituted to form a quaternary ammonium salt. R₅ in the pyrimidine ring may be N or CR, where R is H, halogen containing groups, or alkyl (see, e.g., U.S. Pat. No. 4,086,417). R₆ and R₇ can together can form an oxo group or R₆=—NH—R₁ and R₇=H. R₈ is H or R₇ and R₈ together can form a double bond or R₈ can be X, where X is:

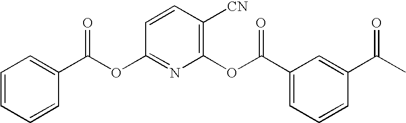

Specific pyrimidine analogs are disclosed in U.S. Pat. No. 3,894,000 (see, e.g., 2′-O-palmityl-ara-cytidine, 3′-O-benzoyl-ara-cytidine, and more than 10 other examples); U.S. Pat. No. 3,991,045 (see, e.g., N4-acyl-1-β-D-arabinofuranosylcytosine, and numerous acyl groups derivatives as listed therein, such as palmitoyl.

In another aspect, the Cell Cycle Inhibitor that is associated with an anastomotic connection device according to the present invention is a Fluoropyrimidine Analog, such as 5-Fluorouracil, or an analog or derivative thereof, including Carmofur, Doxifluridine, Emitefur, Tegafur, and Floxuridine. Exemplary compounds have the structures:

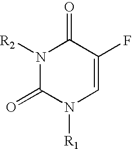

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

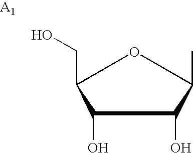

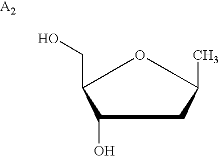

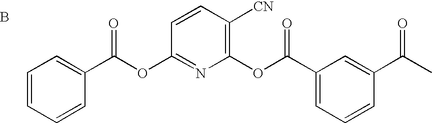

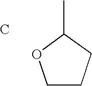

Other suitable Fluoropyrimidine Analogs include 5-FudR (5-fluorodeoxyuridine), or an analog or derivative thereof, including 5-iododeoxyuridine (5-ludR), 5-bromodeoxyuridine (5-BudR), Fluorouridine triphosphate (5-FUTP), and Fluorodeoxyuridine monophosphate (5-dFUMP). Exemplary compounds have the structures:

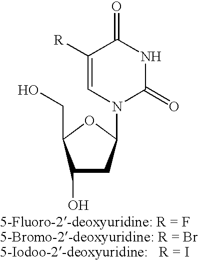

These compounds are thought to function as Cell Cycle Inhibitors by serving as antimetabolites of pyrimidine. These compounds have been shown useful in the treatment of cell proliferative disorders such as breast, cervical, non-melanoma skin, head and neck, esophageal, bile duct, pancreatic, islet cell, penile, and vulvar cancers.

In another aspect, the Cell Cycle Inhibitor that is associated with an anastomotic connection device according to the present invention is a Purine Analog. Purine analogs have the following general structure.

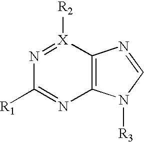

wherein X is typically carbon; R₁ is H, halogen, amine or a substituted phenyl; R₂ is H, a primary, secondary or tertiary amine, a sulfur containing group, typically —SH, an alkane, a cyclic alkane, a heterocyclic or a sugar; R₃ is H, a sugar (typically a furanose or pyranose structure), a substituted sugar or a cyclic or heterocyclic alkane or aryl group. See, e.g., U.S. Pat. No. 5,602,140 for compounds of this type.

In the case of pentostatin, X-R2 is —CH₂CH(OH)—. In this case a second carbon atom is inserted in the ring between X and the adjacent nitrogen atom. The X—N double bond becomes a single bond.

U.S. Pat. No. 5,446,139 describes suitable purine analogs of the type shown in the formula.

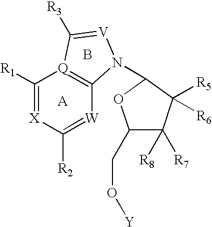

wherein N signifies nitrogen and V, W, X, Z can be either carbon or nitrogen with the following provisos. Ring A may have 0 to 3 nitrogen atoms in its structure. If two nitrogen atoms are present in ring A, one must be in the W position. If only one is present, it must not be in the Q position. V and Q must not be simultaneously nitrogen. Z and Q must not be simultaneously nitrogen. If Z is nitrogen, R₃ is not present. Furthermore, R₁₋₃ are independently one of H, halogen, C₁₋₇ alkyl, C₁₋₇ alkenyl, hydroxyl, mercapto, C₁₋₇ alkylthio, C₁₋₇ alkoxy, C₂₋₇ alkenyloxy, aryl oxy, nitro, primary, secondary or tertiary amine containing group. R₅₋₈ are H or up to two of the positions may contain independently one of OH, halogen, cyano, azido, substituted amino, R₅ and R₇ can together form a double bond. Y is H, a C₁₋₇ alkylcarbonyl, or a mono- di or tri phosphate.

Exemplary suitable purine analogs include 6-Mercaptopurine, Thiguanosine, Thiamiprine, Cladribine, Fludaribine, Tubercidin, Puromycin, Pentoxyfilline; where these compounds may optionally be phosphorylated. Exemplary compounds have the structures:

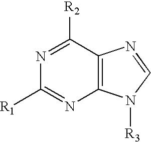

| | R₁ | R₂ | R₃ | | |
|---|---|---|---|---|---|
| 6-Mercaptopurine Thioguanosine | H NH₂ | SH SH | H B₁ | 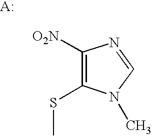 | 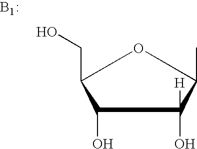 |
| Thiamiprine Cladribine Fludarabine | NH₂Cl F | A NH₂NH₂ | H B₂B₃ | 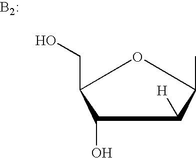 | 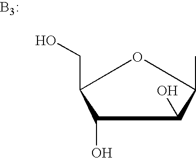 |

| | | | | |
|---|---|---|---|---|
| Puromycin Tubercidin | H H | N(CH₃)₂NH₂ | B₄B₁ | 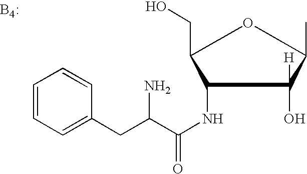 |

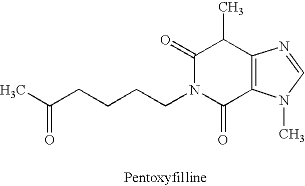

These compounds are thought to function as Cell Cycle Inhibitors by serving as antimetabolites of purine.

In another aspect, the Cell Cycle Inhibitor that is associated with an anastomotic connection device according to the present invention is a Nitrogen Mustard. Many suitable Nitrogen Mustards are known and are suitably used as a Cell Cycle Inhibitor in the present invention. Suitable nitrogen mustards are also known as cyclophosphamides.

A preferred nitrogen mustard has the general structure:

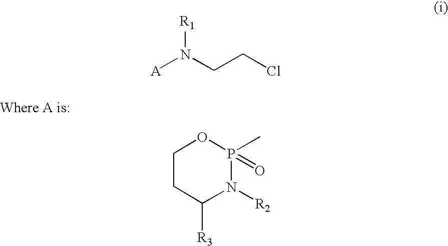

or —CH₃ or other alkane, or chlorinated alkane, typically CH₂CH(CH₃)Cl, or a polycyclic group such as B, or a substituted phenyl such as C or a heterocyclic group such as D.

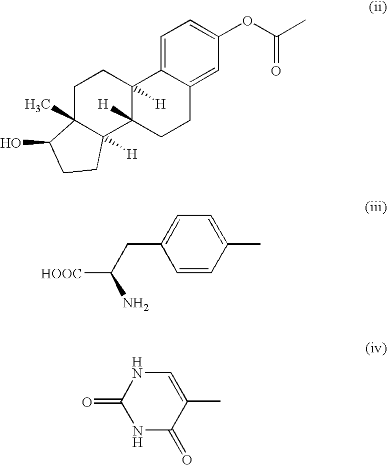

Suitable nitrogen mustards are disclosed in U.S. Pat. No. 3,808,297, wherein A is:

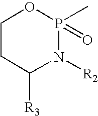

R₁₋₂ are H or CH₂CH₂Cl; R₃ is H or oxygen-containing groups such as hydroperoxy; and R₄ can be alkyl, aryl, heterocyclic.

The cyclic moiety need not be intact. See, e.g., U.S. Pat. Nos. 5,472,956, 4,908,356, 4,841,085 that describe the following type of structure:

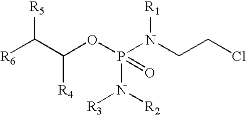

wherein R₁ is H or CH₂CH₂Cl, and R₂₋₆ are various substituent groups.

Exemplary nitrogen mustards include methylchloroethamine, and analogs or derivatives thereof, including methylchloroethamine oxide hydrochloride, Novembichin, and Mannomustine (a halogenated sugar). Exemplary compounds have the structures:

| 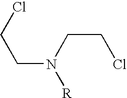 | | |
|---|---|---|
| | R | 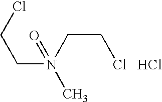 |
| Mechiorethanime | CH₃ | Mechlorethanime Oxide HCl |
| Novembichin | CH₂CH(CH₃)Cl | |

The Nitrogen Mustard may be Cyclophosphamide, Ifosfamide, Perfosfamide, or Torofosfamide, where these compounds have the structures:

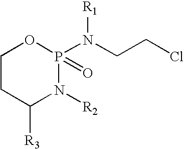

| | R₁ | R₂ | R₃ |
|---|---|---|---|
| Cyclophosphamide | H | CH₂CH₂Cl | H |
| Ifosfamide | CH₂CH₂Cl | H | H |
| Perfosfamide | CH₂CH₂Cl | H | OOH |
| Torofosfamide | CH₂CH₂Cl | CH₂CH₂Cl | H |

The Nitrogen Mustard may be Estramustine, or an analog or derivative thereof, including Phenesterine, Prednimustine, and Estramustine PO₄. Thus, suitable nitrogen mustard type Cell Cycle Inhibitors of the present invention have the structures:

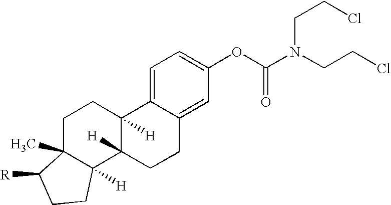

| | R |
|---|---|
| Estramustine | OH |
| Phenesterine | C(CH₃)(CH₂)₃CH(CH₃)₂ |

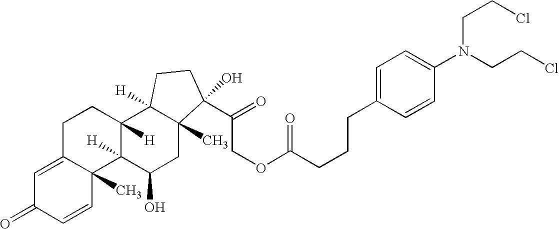

Prednimustine

The Nitrogen Mustard may be Chlorambucil, or an analog or derivative thereof, including Melphalan and Chlormaphazine. Thus, suitable nitrogen mustard type Cell Cycle Inhibitors of the present invention have the structures:

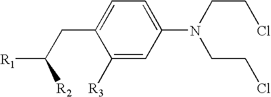

| | R₁ | R₂ | R₃ |
|---|---|---|---|
| Chlorambucil | CH₂COOH | H | H |
| Melphalan | COOH | NH₂ | H |

| | | |
|---|---|---|
| Chlornaphazine | H | together forms a |
| | | benzene ring |

The Nitrogen Mustard may be Uracil Mustard, which has the structure:

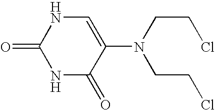

The Nitrogen Mustards are thought to function as Cell Cycle Inhibitors by serving as alkylating agents for DNA. Nitrogen Mustards have been shown useful in the treatment of cell proliferative disorders including, for example, small cell lung, breast, cervical, head and neck, prostate, retinoblastoma, and soft tissue sarcoma.

In another aspect, the Cell Cycle Inhibitor that is associated with an anastomotic connection device according to the present invention is a Hydroxyurea. Hydroxyureas have the following general structure:

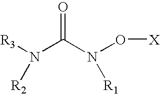

Suitable Hydroxyureas are disclosed in, for example, U.S. Pat. No. 6,080,874, wherein R₁ is:

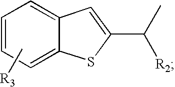

and R₂ is an alkyl group having 1-4 carbons and R₃ is one of H, acyl, methyl, ethyl, and mixtures thereof, such as a methylether.

Other suitable Hydroxyureas are disclosed in, e.g., U.S. Pat. No. 5,665,768, wherein R₁ is a cycloalkenyl group, for example N-[3-[5-(4-fluorophenylthio)-furyl]-2-cyclopenten-1-yl]N-hydroxyurea; R₂ is H or an alkyl group having 1 to 4 carbons and R₃ is H; X is H or a cation.

Other suitable Hydroxyureas are disclosed in, e.g., U.S. Pat. No. 4,299,778, wherein R₁ is a phenyl group substituted with on or more fluorine atoms; R₂ is a cyclopropyl group; and R₃ and X is H.

Other suitable Hydroxyureas are disclosed in, e.g., U.S. Pat. No. 5,066,658, wherein R₂ and R₃ together with the adjacent nitrogen form:

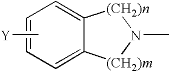

wherein m is 1 or 2, n is 0-2 and Y is an alkyl group.

In one aspect, the hydroxy urea has the structure:

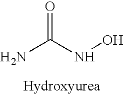

Hydroxyureas are thought to function as Cell Cycle Inhibitors by serving to inhibit DNA synthesis.

In another aspect, the Cell Cycle Inhibitor that is associated with an anastomotic connection device according to the present invention is a Mytomicin, such as Mitomycin C, or an analog or derivative thereof, such as Porphyromycin. Suitable compounds have the structures:

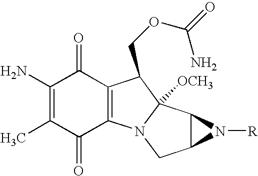

| | R |
|---|---|
| Mitomycin C | H |
| Porphyromycin | CH₃ |
| (N-methyl Mitomycin C) | |

These compounds are thought to function as Cell Cycle Inhibitors by serving as DNA alkylating agents. Mitomycins have been shown useful in the treatment of cell proliferative disorders such as, for example, esophageal, liver, bladder, and breast cancers.

In another aspect, the Cell Cycle Inhibitor that is associated with an anastomotic connection device according to the present invention is an Alkyl sulfonate, such as Busulfan, or an analog or derivative thereof, such as Treosulfan, Improsulfan, Piposulfan, and Pipobroman. Exemplary compounds have the structures:

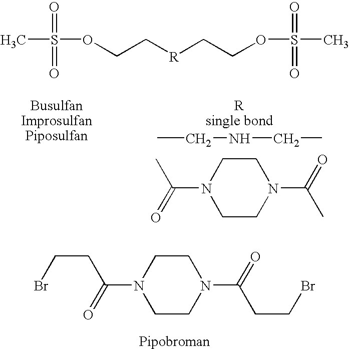

These compounds are thought to function as Cell Cycle Inhibitors by serving as DNA alkylating agents.

In another aspect, the Cell Cycle Inhibitor that is associated with an anastomotic connection device according to the present invention is a Benzamide. In yet another aspect, the Cell Cycle Inhibitor that is associated with an anastomotic connection device according to the present invention is a Nicotinamide. These compounds have the basic structure:

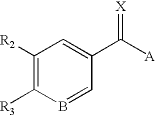

wherein X is either O or S; A is commonly NH₂ or it can be OH or an alkoxy group; B is N or C—R₄, where R₄ is H or an ether-linked hydroxylated alkane such as OCH₂CH₂OH, the alkane may be linear or branched and may contain one or more hydroxyl groups. Alternately, B may be N—R₅ in which case the double bond in the ring involving B is a single bond. R₅ may be H, and alkyl or an aryl group (see, e.g., U.S. Pat. No. 4,258,052); R₂ is H, OR₆, SR₆ or NHR₆, where R₆ is an alkyl group; and R₃ is H, a lower alkyl, an ether linked lower alkyl such as —O-Me or —O-Ethyl (see, e.g., U.S. Pat. No. 5,215,738).

Suitable Benzamide compounds have the structures:

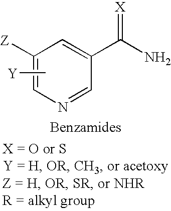

where additional compounds are disclosed in U.S. Pat. No. 5,215,738, (listing some 32 compounds).

Suitable Nicotinamide compounds have the structures:

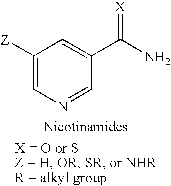

where additional compounds are disclosed in U.S. Pat. No. 5,215,738 (listing some 58 compounds, e.g., 5-OH nicotinamide, 5-aminonicotinamide, 5-(2,3-dihydroxypropoxy) nicotinamide, and compounds having the structures:

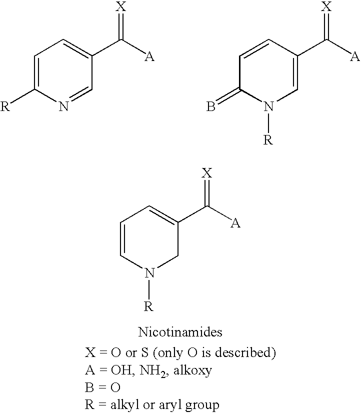

and U.S. Pat. No. 4,258,052 (listing some 46 compounds, e.g., 1-methyl-6-keto-1,6-dihydronicotinic acid).

In one aspect, the Cell Cycle Inhibitor that is associated with an anastomotic connection device according to the present invention is a Tetrazine Compound, such as Temozolomide, or an analog or derivative thereof, including Dacarbazine. Suitable compounds have the structures:

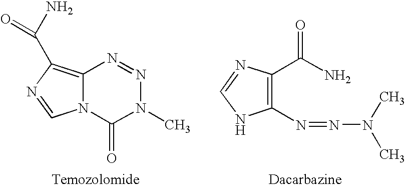

Another suitable Tetrazine Compound is Procarbazine, including HCl and HBr salts, having the structure:

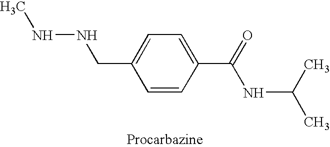

In another aspect, the Cell Cycle Inhibitor that is associated with an anastomotic connection device according to the present invention is Actinomycin D, or other members of this family, including Dactinomycin, Actinomycin C₁, Actinomycin C₂, Actinomycin C₃, and Actinomycin F₁. Suitable compounds have the structures:

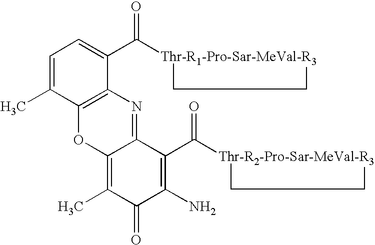

| | R₁ | R₂ | R₃ |
|---|---|---|---|
| Actinomycin D (C₁) | D-Val | D-Val | single bond |
| Actinomycin C₂ | D-Val | D-Alloisoleucine | O |
| Actinomycin C₃ | D-Alloisoleucine | D-Alloisoleucine | O |

In another aspect, the Cell Cycle Inhibitor that is associated with an 5 anastomotic connection device according to the present invention is an Aziridine compound, such as Benzodepa, or an analog or derivative thereof, including Meturedepa, Uredepa, and Carboquone. Suitable compounds have the structures:

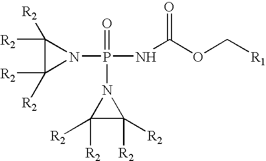

| | R₁ | R₂ |
|---|---|---|
| Benzodepa | phenyl | H |
| Meturedepa | CH₃ | CH₃ |
| Uredepa | CH₃ | H |

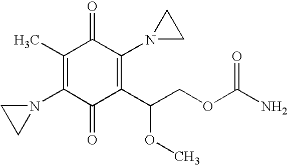

Carboquone

In another aspect, the Cell Cycle Inhibitor that is associated with an anastomotic connection device according to the present invention is Halogenated Sugar, such as Mitolactol, or an analog or derivative thereof, including Mitobronitol and Mannomustine. Suitable compounds have the structures:

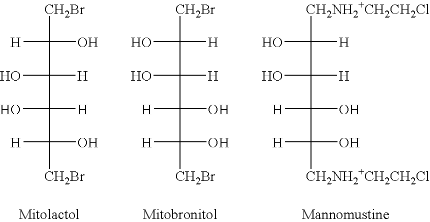

In another aspect, the Cell Cycle Inhibitor that is associated with an anastomotic connection device according to the present invention is a Diazo compound, such as Azaserine, or an analog or derivative thereof, including 6-diazo-5-oxo-L-norleucine and 5-diazouracil (also a pyrimidine analog). Suitable compounds have the structures:

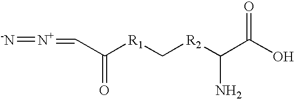

| | R₁ | R₂ |
|---|---|---|
| Azaserine | O | single bond |
| 6-diazo-5-oxo- | single bond | CH₂ |
| L-norleucine | | |

Other compounds that may serve as Cell Cycle Inhibitors and may be placed in association with an anastomotic connection device according to the present invention are Pazelliptine; Wortmannin; Metoclopramide; RSU; Buthionine sulfoxime; Tumeric; Curcumin; AG337, a thymidylate synthase inhibitor; Levamisole; Lentinan, a polysaccharide; Razoxane, an EDTA analog; Indomethacin; Chlorpromazine; α and β interferon; MnBOPP; Gadolinium texaphyrin; 4-amino-1,8-naphthalimide; Staurosporine derivative of CGP; and SR-2508.

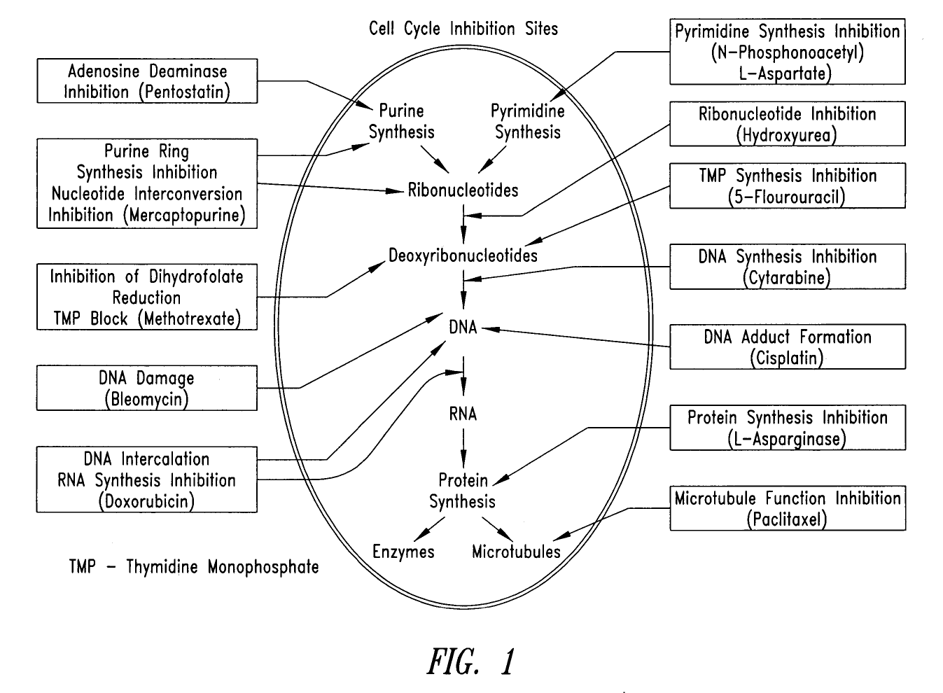

Thus, in one aspect, the Cell Cycle Inhibitor that is associated with an anastomotic connection device according to the present invention is a DNA alkylating agent. In another aspect, the Cell Cycle Inhibitor that is associated with an anastomotic connection device according to the present invention is an anti-microtubule agent. In another aspect, the Cell Cycle Inhibitor that is associated with an anastomotic connection device according to the present invention is a Topoisomerase inhibitor. In another aspect, the Cell Cycle Inhibitor that is associated with an anastomotic connection device according to the present invention is a DNA cleaving agent. In another aspect, the Cell Cycle Inhibitor that is associated with an anastomotic connection device according to the present invention is an antimetabolite. In another aspect, the Cell Cycle Inhibitor that is associated with an anastomotic connection device according to the present invention functions by inhibiting adenosine deaminase (e.g., as a purine analog). In another aspect, the Cell Cycle Inhibitor that is associated with an anastomotic connection device according to the present invention functions by inhibiting purine ring synthesis and/or as a nucleotide interconversion inhibitor (e.g., as a purine analog such as mercaptopurine). In another aspect, the Cell Cycle Inhibitor that is associated with an anastomotic connection device according to the present invention functions by inhibiting dihydrofolate reduction and/or as a thymidine monophosphate block (e.g., methotrexate). In another aspect, the Cell Cycle Inhibitor that is associated with an anastomotic connection device according to the present invention functions by causing DNA damage (e.g., Bleomycin). In another aspect, the Cell Cycle Inhibitor that is associated with an anastomotic connection device according to the present invention functions as a DNA intercalation agent and/or RNA synthesis inhibition (e.g., Doxorubicin). In another aspect, the Cell Cycle Inhibitor that is associated with an anastomotic connection device according to the present invention functions by inhibiting pyrimidine synthesis (e.g., N-phosphonoacetyl-L-Aspartate). In another aspect, the Cell Cycle Inhibitor that is associated with an anastomotic connection device according to the present invention functions by inhibiting ribonucleotides (e.g., hydroxyurea). In another aspect, the Cell Cycle Inhibitor that is associated with an anastomotic connection device according to the present invention functions by inhibiting thymidine monophosphate (e.g., 5-fluorouracil). In another aspect, the Cell Cycle Inhibitor that is associated with an anastomotic connection device according to the present invention functions by inhibiting DNA synthesis (e.g., Cytarabine). In another aspect, the Cell Cycle that is associated with an anastomotic connection device according to the present invention Inhibitor functions by causing DNA adduct formation (e.g., platinum compounds). In another aspect, the Cell Cycle Inhibitor that is associated with an anastomotic connection device according to the present invention functions by inhibiting protein synthesis (e.g., L-Asparginase). In another aspect, the Cell Cycle Inhibitor that is associated with an anastomotic connection device according to the present invention functions by inhibiting microtubule function (e.g., taxanes). In another aspect, the Cell Cycle Inhibitor that is associated with an anastomotic connection device according to the present invention acts at one or more of the steps in the biological pathway shown in FIG. 1.

Additional Cell Cycle Inhibitors useful in the present invention, as well as a discussion of their mechanisms of action, may be found in Hardman J. G., Limbird L. E. Molinoff R. B., Ruddon R W., Gilman A. G. editors, Chemotherapy of Neoplastic Diseases in Goodman and Gilman's The Pharmacological Basis of Therapeutics Ninth Edition, McGraw-Hill Health Professions Division, New York, 1996, pages 1225-1287. See also U.S. Pat. Nos. 3,387,001; 3,808,297; 3,894,000; 3,991,045; 4,012,390; 4,057,548; 4,086,417; 4,144,237; 4,150,146; 4,210,584; 4,215,062; 4,250,189; 4,258,052; 4,259,242; 4,296,105; 4,299,778; 4,367,239; 4,374,414; 4,375,432; 4,472,379; 4,588,831; 4,639,456; 4,767,855; 4,828,831; 4,841,045; 4,841,085; 4,908,356; 4,923,876; 5,030,620; 5,034,320; 5,047,528; 5,066,658; 5,166,149; 5,190,929; 5,215,738; 5,292,731; 5,380,897; 5,382,582; 5,409,915; 5,440,056; 5,446,139; 5,472,956; 5,527,905; 5,552,156; 5,594,158; 5,602,140; 5,665,768; 5,843,903; 6,080,874; 6,096,923; and RE030561.

In one embodiment the cell-cycle inhibitor that is associated with an anastomotic connection device according to the present invention is camptothecin, mitoxantrone, etoposide, 5-fluorouracil, doxorubicin, methotrexate, peloruside A, Mitomycin C, or a CDK-2 inhibitor or an analogue or derivative of any member of the class of listed compounds.

Other examples of cell cycle inhibitors that may be associated with an anastomotic connection device according to the present invention include, e.g. 7-hexanoyltaxol (QP-2), cytochalasin A ,lantrunculin D, actinomycin-D, Ro-31-7453 (3-[6-Nitro-1-methyl-3-indolyl]-4-[1-methyl-3-indolyl]pyrrole-2,5-dione), PNU-151807, brostallicin, C2-ceramide, cytarabine ocfosfate (2(1H)-Pyrimidinone, 4-amino-1-[5-O-[hydroxy(octadecyloxy)phosphinyl]-β-D-arabinofuranosyl]-, monosodium salt), paclitaxel (5β,20-Epoxy-1,2Alpha,4,7β,10β,13Alpha-hexahydroxytax-11-en-9-one-4,10-diacetate-2-benzoate-13-(Alpha-phenylhippurate)), doxorubicin (5,12-Naphthacenedione, 10-[(3-amino-2,3,6-trideoxy-Alpha-L-lyxo-hexopyranosyl)oxy]-7,8,9,10-tetrahydro-6,8,11-trihydroxy-8-(hydroxyacetyl)-1-methoxy-, (8S)-cis-), daunorubicin (5,12-Naphthacenedione, 8-acetyl-10-[(3-amino-2,3,6-trideoxy-Alpha-L-lyxo-hexopyranosyl)oxy]-7,8,9,10-tetrahydro-6,8,11-trihydroxy-1-methoxy-, (8S-cis)-), gemcitabine hydrochloride (Cytidine, 2′-deoxy-2′,2′-difluoro-,monohydrochloride ), nitacrine (1,3-Propanediamine, N,N-dimethyl-N′-(1-nitro-9-acridinyl)-), carboplatin (Platinum, diamine[1,1-cyclobutanedicarboxylato(2-)]-, (SP-4-2)-), altretamine (1,3,5-Triazine-2,4,6-triamine, N,N,N′,N′,N″,N″-hexamethyl-), teniposide (Furo[3′,4′:6,7]naphtho[2,3-d]-1,3-dioxol-6(5aH)-one, 5,8,8a,9-tetrahydro-5-(4-hydroxy-3,5-dimethoxyphenyl)-9-[[4,6-O-(2-thienylmethylene)-β-D-glucopyranosyl]oxy]-, [5R-[5Alpha,5aβ,8aAlpha,9β(R*)]]-), eptaplatin (Platinum, [(4R,5R)-2-(1-methylethyl)-1,3-dioxolane-4,5-dimethanamine-kappaN4,kappaN5][propanedioato(2-)-kappaO1,kappaO3]-, (SP-4-2)-), amrubicin hydrochloride (5,12-Naphthacenedione, 9-acetyl-9-amino-7-[(2-deoxy-β-D-erythropentopyranosyl)oxy]-7,8,9,10-tetrahydro-6 11-dihydroxy-, hydrochloride, (7S-cis)-), ifosfamide (2H-1,3,2-Oxazaphosphorin-2-amine, N,3-bis(2-chloroethyl)tetrahydro-,2-oxide ), cladribine (Adenosine, 2-chloro-2′-deoxy-), mitobronitol (D-Mannitol, 1,6-dibromo-1,6-dideoxy-), fludaribine phosphate (9H-Purin-6-amine, 2-fluoro-9-(5-O-phosphono-β-D-arabinofuranosyl)-), enocitabine (Docosanamide, N-(1-β-D-arabinofuranosyl-1,2-dihydro-2-oxo-4-pyrimidinyl)-), vindesine (Vincaleukoblastine, 3-(aminocarbonyl)-O4-deacetyl-3-de(methoxycarbonyl)-), idarubicin (5,12-Naphthacenedione, 9-acetyl-7-[(3-amino-2,3,6-trideoxy-Alpha-L-lyxo-hexopyranosyl)oxy]-7,8,9,10-tetrahydro-6,9,11-trihydroxy-, (7S-cis)-), zinostatin (Neocarzinostatin ), vincristine (Vincaleukoblastine, 22-oxo-), tegafur (2,4(1H,3H)-Pyrimidinedione, 5-fluoro-1-(tetrahydro-2-furanyl)-), razoxane (2,6-Piperazinedione, 4,4′-(1-methyl-1,2-ethanediyl)bis-), methotrexate (L-Glutamic acid, N-[4-[[(2,4-diamino-6-pteridinyl)methyl]methylamino]benzoyl]-), raltitrexed (L-glutamic acid, N-[[5-[[(1,4-dihydro-2-methyl-4-oxo-6-quinazolinyl)methyl]methylamino]-2-thienyl]carbonyl]-), oxaliplatin (Platinum, (1,2-cyclohexanediamine-N,N′)[ethanedioato(2-)-O,O′]-, [SP-4-2-(1R-trans)]-), doxifluridine (Uridine, 5′-deoxy-5-fluoro-), mitolactol (Galactitol, 1,6-dibromo-1,6-dideoxy-), piraubicin (5,12-Naphthacenedione, 10-[[3-amino-2,3,6-trideoxy-4-O-(tetrahydro-2H-pyran-2-yl)-Alpha-L-Iyxo-hexopyranosyl]oxy]-7,8,9,10-tetrahydro-6,8,11-trihydroxy-8-(hydroxyacetyl)-1-methoxy-, [8S-[8Alpha,10Alpha(S*)]]-), docetaxel ((2R,3S)-N-Carboxy-3-phenylisoserine, N-tert-butyl ester, 13-ester with 5β,20-epoxy-1,2Alpha,4,7β,10β,13Alpha-hexahydroxytax-11-en-9-one 4-acetate 2-benzoate-), capecitabine (Cytidine, 5-deoxy-5-fluoro-N-[(pentyloxy)carbonyl]-), cytarabine (2(1H)-Pyrimidone, 4-amino-1-β-D-arabino furanosyl-), valrubicin (Pentanoic acid, 2-(1,2,3,4,6,11-hexahydro-2,5,12-trihydroxy-7-methoxy-6,11-dioxo-4-((2,3,6-trideoxy-3-((trifluoroacetyl)amino)-Alpha-L-lyxo-hexopyranosyl)oxy)-2-naphthacenyl)-2-oxoethyl ester (2S-cis)-), trofosfamide (3-2-(chloroethyl)-2-[bis(2-chloroethyl)amino]tetrahydro-2H-1,3,2-oxazaphosphorin 2-oxide), prednimustine (Pregna-1,4-diene-3,20-dione, 21-[4-[4-[bis(2-chloroethyl)amino]phenyl]-1-oxobutoxy]-11,17-dihydroxy-, (11β)-), lomustine (Urea, N-(2-chloroethyl)-N′-cyclohexyl-N-nitroso-), epirubicin (5,12-Naphthacenedione, 10-[(3-amino-2,3,6-trideoxy-Alpha-L-arabino-hexopyranosyl)oxy]-7,8,9,10-tetrahydro-6,8,11-trihydroxy-8-(hydroxyacetyl)-1-methoxy-, (8S-cis)-), or an analogue or derivative thereof.

As mentioned above, the present invention provides that each of the afore-mentioned classes of cell cycle inhibitors may be placed in association with an anastomotic connection device. The following sections identify and describe compounds that, in separate aspects of the invention, are associated with an anastomotic connector device. In some instances, the compounds described below can function as cell cycle inhibitors, and are also discussed in the present section directed to cell cycle inhibitors.

D. Anthracyclines

In one aspect of the present invention, an anastomotic connection device is therapeutically associated with an anthracycline, where anthracyclines have the following general structure, where the R groups may be a variety of organic groups:

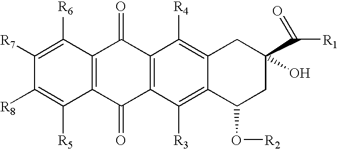

As set forth in U.S. Pat. No. 5,594,158, suitable R groups are as follows: R₁ is CH₃ or CH₂OH; R₂ is daunosamine or H; R₃ and R₄ are independently one of OH, NO₂, NH₂, F, Cl, Br, I, CN, H or groups derived from these; R₅ is hydrogen, hydroxy, or methoxy; and R₆₋₈ are all hydrogen. Alternatively, R₅ and R₆ are hydrogen and R₇ and R₈ are alkyl or halogen, or vice versa.

As set forth in U.S. Pat. No. 5,843,903, R₁ may be a conjugated peptide. Of U.S. Pat. No. 4,296,105, R₅ may be an ether linked alkyl group. Of U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as—CH₂CH(CH₂-X)C(O)-R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure:

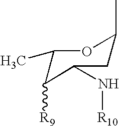

in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxy, mercapto, phenyl, benzyl or methylthio (see U.S. Pat. No. 4,296,105).

Exemplary anthracyclines are Doxorubicin, Daunorubicin, Idarubicin, Epirubicin, Pirarubicin, Zorubicin, and Carubicin. Suitable compounds have the structures:

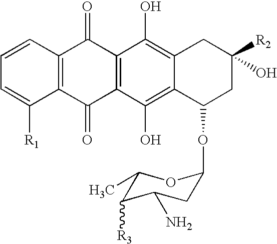

| | R₁ | R₂ | R₃ |
|---|---|---|---|
| Doxorubicin: | OCH₃ | C(O)CH₂OH | OH out of ring plane |
| Epirubicin: | OCH₃ | C(O)CH₂OH | OH in ring plane |
| (4′ epimer of | | | |
| doxorubicin) | | | |
| Daunorubicin: | OCH₃ | C(O)CH₃ | OH out of ring plane |
| Idarubicin: | H | C(O)CH₃ | OH out of ring plane |
| Pirarubicin: | OCH₃ | C(O)CH₂OH | 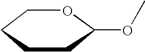 |
| Zorubicin: | OCH₃ | C(CH₃)(═N)NHC(O)C₆H₅ | OH |
| Carubicin: | OH | C(O)CH₃ | OH out of ring plane |

Other suitable anthracyclines are Anthramycin, Mitoxantrone, Menogaril, Nogalamycin, Aclacinomycin A, Olivomycin A, Chromomycin A₃, and Plicamycin having the structures:

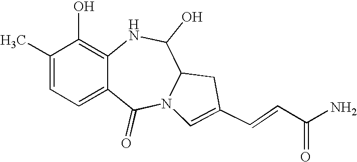

Anthramycin

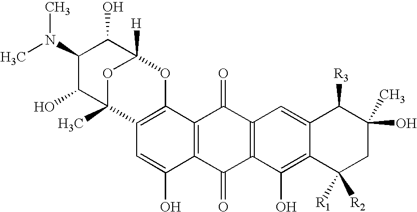

| | R₁ | R₂ | R₃ |
|---|---|---|---|
| Menogaril | H | OCH₃ | H |
| Nogalamycin | O-sugar | H | COOCH₃ |
| sugar: | | 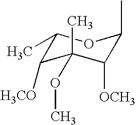 | |

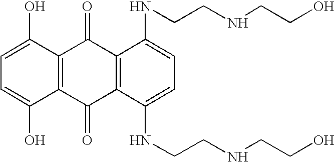

Mitoxantrone

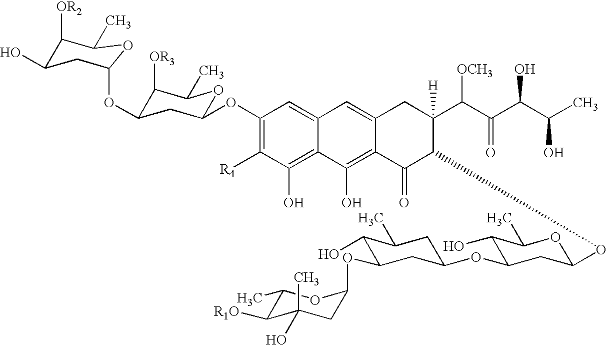

| | R₁ | R₂ | R₃ | R₄ |
|---|---|---|---|---|
| Otivomycin A | COCH(CH₃)₂ | CH₃ | COCH₃ | H |
| Chromomycin A₃ | COCH₃ | CH₃ | COCH₃ | CH₃ |
| Plicamycin | H | H | H | CH₃ |

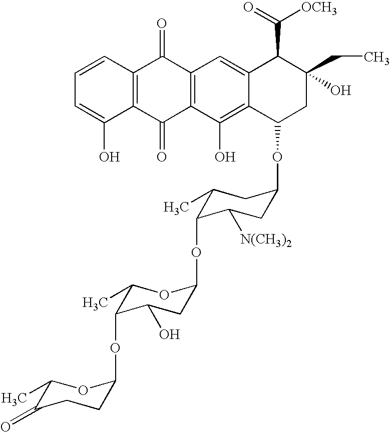

Adacinomycin A

Other representative anthracyclines include, FCE 23762 doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11 ):2833-2839, 1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Nat'l Cancer Inst. 89(16):1217-1223, 1997), 4-demethoxy-7-O-[2,6-dideoxy-4-O-(2,3,6-trideoxy-3-amino- □-L-lyxo-hexopyranosyl)-□-L-lyxo-hexopyranosyl]adriamicinone doxorubicin disaccharide analogue (Monteagudo et al., Carbohydr. Res. 300(1):11-16, 1997), 2-pyrrolinodoxorubicin (Nagy et al., Proc. Nat'l Acad. Sci. U.S.A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int. J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Willner et al., Bioconjugate Chem. 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ., 16(Biol. 1):21-7, 1988), 4′-deoxydoxorubicin (Schoelzel et al., Leuk. Res. 10(12):1455-9, 1986), 4-demethyoxy-4′-o-methyldoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyidoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20, 1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277).

E. Taxanes

In one aspect of the present invention, an anastomotic connection device is therapeutically associated with a taxane, or a derivative or an analogue thereof. Briefly, taxanes such as, for example, paclitaxel, are compounds that disrupt mitosis (M-phase) by binding to tubulin to form abnormal mitotic spindles.

The taxane paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216, 1993). It has been formulated into commercial compositions, including the product TAXOL®. Analogues and derivatives of paclitaxel include, for example, commercial products such as TAXOTERE®, as well as compounds such as docetaxel, 10-desacetyl analogues of paclitaxel and 3′N-desbenzoyl-3′N-t-butoxy carbonyl analogues of paclitaxel) (see generally Schiff et al., Nature 277:665-667, 1979; Long and Fairchild, Cancer Research 54:4355-4361, 1994; Ringel and Horwitz, J. Nat'l Cancer lnst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988). Taxanes can be made utilizing the techniques cited within the references provided herein, or obtained from a variety of commercial sources, including for example, Sigma Chemical Co., St. Louis, Mo. (T7402—from Taxus brevifolia).

Further representative examples of taxanes include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′-and/or 7-O-ester derivatives ), (2′-and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogues with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogues bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the taxane has the formula (C1):

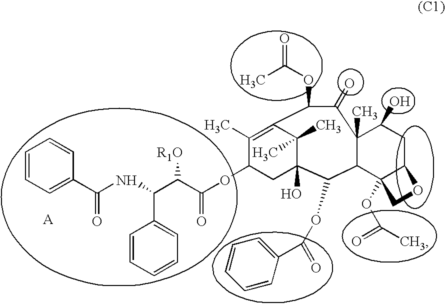

where the gray-highlighted portions may be substituted and the non-highlighted portion is the taxane core. A side-chain (labeled “A” in the diagram) is desirably present in order for the compound to have good activity. Examples of compounds having this structure include paclitaxel (Merck Index entry 7117), docetaxel (Taxotere, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogues and derivatives are disclosed in U.S. Pat. No. 5,440,056 as having the structure (C2):

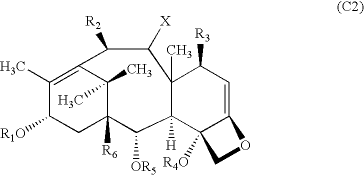

wherein X may be oxygen (paclitaxel), hydrogen (9-deoxy derivatives), thioacyl, or dihydroxyl precursors; R₁ is selected from paclitaxel or taxotere side chains or alkanoyl of the formula (C3)

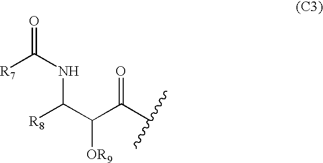

wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogues and derivatives useful in the present invention are disclosed in PCT International Patent Application No. WO 93/10076. As disclosed in this publication, the analogue or derivative should have a side chain attached to the taxane nucleus at C₁₃, as shown in the structure below (formula C4), in order to confer antitumor activity to the taxane.

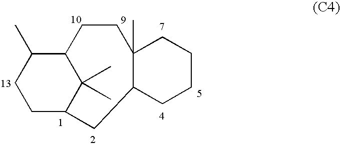

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, taxanes which are useful in the present invention are disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aroyl, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

F. Cyclin Dependent Protein Kinase Inhibitors

In one aspect of the present invention, an anastomotic connection device is therapeutically associated with a cyclin dependent protein kinase inhibitor, where exemplary compounds having this biological activity include: R-roscovitine, CYC-101, CYC-103, CYC-400, MX-7065, alvocidib (4H-1-Benzopyran-4-one, 2-(2-chlorophenyl)-5,7-dihydroxy-8-(3-hyd roxy- 1-methyl-4-piperidinyl)-, cis-(-)-), SU-9516, AG-12275, PD-0166285, CGP-79807, fascaplysin, GW-8510 (Benzenesulfonamide, 4-[[(Z)-(6,7-dihydro-7-oxo-8H-pyrrolo[2,3-g]benzothiazol-8-ylidene)methyl]amino]-N-(3-hydroxy-2,2-dimethylpropyl)-), GW-491619, Indirubin 3′monoxime, GW8510, or an analogue or derivative thereof.

G. EGF (Epidermal Growth Factor) Receptor Kinase Inhibitors

In one aspect of the present invention, an anastomotic connection device is therapeutically associated with an EGF (epidermal growth factor) kinase inhibitor, where exemplary compounds having this biological activity include Erlotinib (4-Quinazolinamine, N-(3-ethynylphenyl)-6,7-bis(2-methoxyethoxy)-, monohydrochloride ), Viatris, erbstatin, BIBX-1382, gefitinib (4-Quinazolinamine, N-(3-chloro-4-fluorophenyl)-7-methoxy-6-(3-(4-morpholinyl)propoxy)), or an analogue or derivative thereof.

H. Elastase Inhibitors

In one aspect of the present invention, an anastomotic connection device is therapeutically associated with an elastase inhibitor, where exemplary compounds having this biological activity include: ONO-6818, sivelestat sodium hydrate (Glycine, N-[2-[[[4-(2,2-dimethyl-1-oxopropoxy)phenyl]sulfonyl]amino]benzoyl]-), erdosteine (Acetic acid, [[2-oxo-2-[(tetrahydro-2-oxo-3-thienyl)amino]ethyl]thio]-), MDL-100948A, MDL-104238 (N-[4-(4-morpholinylcarbonyl)benzoyl]-L-valyl-N′-[3,3,4,4,4-pentafluoro-1-(1-methylethyl)-2-oxobutyl]-L-2-azetamide), MDL-27324 (L-Prolinamide, N-[[5-(dimethylamino)-1-naphthalenyl]sulfonyl]-L-alanyl-L-alanyl-N-[3,3,3-trifluoro-1-(1-methylethyl)-2-oxopropyl]-, (S)-), SR-26831 (Thieno[3,2-c]pyridinium, 5-[(2-chlorophenyl)methyl]-2-(2,2-dimethyl-1-oxopropoxy)-4,5,6,7-tetrahydro-5-hydroxy-), Win-68794, Win-63110, SSR-69071 (2-(9(2-Piperidinoethoxy)-4-oxo-4H-pyrido[1,2-a]pyrimidin-2-yloxymethyl)-4-(1-methylethyl)-6-methyoxy-1,2-benzisothiazol-3(2H)-one-1,1-dioxide), (N(Alpha)-(1-adamantylsulfonyl)N(epsilon)-succinyl-L-lysyl-L-prolyl-L-valinal), Ro-31-3537 (NAlpha-(1-adamantanesulphonyl)-N-(4-carboxybenzoyl)-L-lysyl-alanyl-L-valinal), R-665, FCE-28204, ((6R,7R)-2-(Benzoyloxy)-7-methoxy-3-methyl-4-pivaloyl-3-cephem 1,1-dioxide), 1,2-Benzisothiazol-3(2 H)-one, 2-(2,4-dinitrophenyl)-, 1,1-dioxide [CAS], L-658758 (L-Proline, 1-[[3-[(acetyloxy)methyl]-7-methoxy-8-oxo-5-thia-1-azabicyclo[4.2.0]oct-2-en-2-yl]carbonyl]-, S,S-dioxide, (6R-cis)-), L-659286 (Pyrrolidine, 1-[[7-methoxy-8-oxo-3-[[(1,2,5,6-tetrahydro-2-methyl-5,6-dioxo-1,2,4-triazin-3-yl)thio]methyl]-5-thia-1-azabicyclo[4.2.0]oct-2-en-2-yl]carbonyl]-, S,S-dioxide, (6R-cis)-), L-680833 (Benzeneacetic acid, 4-[[3,3-diethyl-1-[[[1-(4-methylphenyl)butyl]amino]carbonyl]-4-oxo-2-azetidinyl]oxy]-, [S-(R*,S*)]-) , or an analogue or derivative thereof.

I. Factor Xa Inhibitors

In one aspect of the present invention, an anastomotic connection device is therapeutically associated with a factor Xa inhibitor, where exemplary compounds having this biological activity include: CY-222, fondaparinux sodium (Alpha-D-Glucopyranoside, methyl O-2-deoxy-6-O-sulfo-2-(sulfoamino)-Alpha-D-glucopyranosyl-(1-4)-O-β-D-glucopyranuronosyl-(1-4)-O-2-deoxy-3,6-di-O-sulfo-2-(sulfoamino)-Alpha-D-glucopyranosyl-(1-4)-O-2-O-sulfo-Alpha-L-idopyranuronosyl-(1-4)-2-deoxy-2-(sulfoamino)-, 6-(hydrogen sulfate) ), danaparoid sodium, or an analogue or derivative thereof.

J. Farnesyltransferase Inhibitors

In one aspect of the present invention, an anastomotic connection device is therapeutically associated with a farnesyltransferase inhibitor, where exemplary compounds having this biological activity include: dichlorobenzoprim (2,4-diamino-5-[4-(3,4-dichlorobenzylamino)-3-nitrophenyl]-6-ethylpyrimidine), B-581, B-956 (N-[8(R)-Amino-2(S )-benzyl-5(S)-isopropyl-9-sulfanyl-3(Z),6(E)-nonadienoyl]-L-methionine), OSI-754, perillyl alcohol (1-Cyclohexene-1-methanol, 4-(1-methylethenyl)- [CAS], RPR-114334, Ionafarnib (1-Piperidinecarboxamide, 4-[2-[4-[(11R)-3,10-dibromo-8-chloro-6,11-dihydro-5H-benzo[5,6]cyclohepta[1,2-b]pyridin-11-yl]-1-piperidinyl]-2-oxoethyl]-), Sch-48755, Sch-226374, (7,8-Dichloro-5H-dibenzo[b,e][1,4]diazepin-11-yl)-pyridin-3-ylmethylamine, J-104126, L-639749, L-731734 (Pentanamide, 2-[[2-[(2-amino-3-mercaptopropyl)amino]-3-methylpentyl]amino]-3-methyl-N-(tetrahydro-2-oxo-3-furanyl)-, [3S-[3R*[2R*[2R*(S*),3S*],3R*]]]-), L-744832 (Butanoic acid, 2-((2-((2-((2-amino-3-mercaptopropyl)amino)-3-methylpentyl)oxy)-1-oxo-3-phenylpropyl)amino)-4-(methylsulfonyl)-, 1-methylethyl ester, (2S-(1(R*(R*)),2R*(S*),3R*))-), L-745631 (1-Piperazinepropanethiol, β-amino-2-(2-methoxyethyl )-4-(1-naphthalenylcarbonyl)-, (βR,2S)-), N-acetyl-N-naphthylmethyl-2(S)-[(1-(4-cyanobenzyl)-1H-imidazol-5-yl)acetyl]amino-3(S)-methylpentamine, (2Alpha)-2-hyd roxy-24,25-dihyd roxylanost-8-en-3-one, BMS-316810, UCF-1-C (2,4-Decadienamide, N-(5-hydroxy-5-(7-((2-hydroxy-5-oxo-1-cyclopenten-l-yl)amino-oxo-1,3,5-heptatrienyl)-2-oxo-7-oxabicyclo(4,1,0)hept-3-en-3-yl)-2,4,6-trimethyl-, (1 S-(1Alpha,3(2E,4E,6S*),5Alpha,5(1E,3E,5E),6Alpha))-), UCF-116-B, or an analogue or derivative thereof.

K. Fibrinogen Antagonists

In one aspect of the present invention, an anastomotic connection device is therapeutically associated with a fibrinogen antagonist, where exemplary compounds having this biological activity include: 2(S)-[(p-Toluenesulfonyl)amino]-3-[[[5,6,7,8,-tetrahydro-4-oxo-5-[2-(piperid in-4-yl)ethyl]-4H-pyrazolo-[1,5-a][1,4]diazepin-2-yl]carbonyl]-amino]propionic acid, streptokinase (Kinase (enzyme-activating), strepto-), urokinase (Kinase (enzyme-activating), uro-), plasminogen activator, pamiteplase, monteplase, heberkinase, anistreplase, alteplase, pro-urokinase, picotamide (1,3-Benzenedicarboxamide, 4-methoxy-N,N′-bis(3-pyridinylmethyl)-), or an analogue or derivative thereof.

L. Guanylate Cyclase Stimulants

In one aspect of the present invention, an anastomotic connection device is therapeutically associated with a guanylate cyclase stimulant, where exemplary compounds having this biological activity include: isosorbide-5-mononitrate (D-Glucitol, 1,4:3,6-dianhydro-, 5-nitrate ), or an analogue or derivative thereof.

M. Heat Shock Protein 90 Antagonists

In one aspect of the present invention, an anastomotic connection device is therapeutically associated with a heat shock protein 90 antagonist, where exemplary compounds having this biological activity include: geldanamycin; NSC-33050 (17-Allylaminogeldanamycin), rifabutin (Rifamycin XIV, 1′,4-didehydro-1-deoxy-1,4-dihydro-5′-(2-methylpropyl)-1-oxo-), 17AAG, or an analogue or derivative thereof.

N. HMGCoA Reductase Inhibitors

In one aspect of the present invention, an anastomotic connection device is therapeutically associated with an HMGCOA reductase inhibitor, where exemplary compounds having this biological activity include: BCP-671, BB-476, fluvastatin (6-Heptenoic acid, 7-[3-(4-fluorophenyl)-1-(1-methylethyl)-1H-indol-2-yl]-3,5-dihydroxy-, monosodium salt, [R*,S*-(E)]-(±)-), dalvastatin (2H-Pyran-2-one, 6-(2-(2-(2-(4-fluoro-3-methylphenyl)-4,4,6,6-tetramethyl-1-cyclohexen-1-yl)ethenyl)tetrahydro)-4-hydroxy-, (4α,6β(E))-(+/−)-), glenvastatin (2H-Pyran-2-one, 6-[2-[4-(4-fluorophenyl)-2-(1-methylethyl)-6-phenyl-3-pyridinyl]ethenyl]tetrahydro-4-hydroxy-, [4R-[4α,6β(E)]]-), S-2468, N-(1-oxododecyl)-4Alpha,10-dimethyl-8-aza-trans-decal-3β-ol, atorvastatin calcium (1H-Pyrrole-1-heptanoic acid, 2-(4-fluorophenyl)-β,δ-dihydroxy-5-(1-methylethyl)-3-phenyl-4-[(phenylamino)carbonyl]-, calcium salt [R-(R*,R*)]-), CP-83101 (6,8-Nonadienoic acid, 3,5-dihydroxy-9,9-diphenyl-, methyl ester, [R*,S*-(E)]-(+/−)-), pravastatin (1-Naphthaleneheptanoic acid, 1,2,6,7,8,8a-hexahydro-β,delta,6-trihydroxy-2-methyl-8-(2-methyl-1-oxobutoxy)-, monosodium salt, [1S-[1βS*,δS*), 2α,6α,8β(R*),8aα]]-), U-20685, pitavastatin (6-Heptenoic acid, 7-[2-cyclopropyl-4-(4-fluorophenyl)-3-quinolinyl]-3,5-dihydroxy-, calcium salt (2:1), [S-[R*,S*-(E)]]-), N-((1-methylpropyl)carbonyl)-8-[2-(tetrahydro-4-hydroxy-6-oxo-2H-pyran-2-yl)ethyl]-perhydro-isoquinoline, dihydromevinolin (Butanoic acid, 2-methyl-, 1,2,3,4,4a,7,8,8a-octahydro-3,7-dimethyl-8-[2-(tetrahydro-4-hydroxy-6-oxo-2H-pyran-2-yl)ethyl]-1-naphthalenyl ester[1Alpha(R*),3α,4aα,7β,8β(2S*,4S*),8aβ]]-), HBS-107, dihydromevinolin (Butanoic acid, 2-methyl-, 1,2,3,4,4a ,7,8,8a-octahydro-3,7-dimethyl-8-[2-(tetrahydro-4-hydroxy-6-oxo-2H-pyran-2-yl)ethyl]-1-naphthalenyl ester[1Alpha(R*),3α,4aα,7β,8β(2S*,4S*),8β]]-), L-669262 (Butanoic acid, 2,2-dimethyl-, 1,2,6,7,8,8a-hexahydro-3,7-dimethyl-6-oxo-8-[2-(tetrahydro-4-hydroxy-6-oxo-2H-pyran-2-yl)ethyl]-1-naphthalenyl[1S-[1α,7β,8β(2S*,4S*),8aβ]]-), simvastatin (Butanoic acid, 2,2-dimethyl-, 1,2,3,7,8,8a-hexahydro-3,7-dimethyl-8-[2-(tetrahydro-4-hydroxy-6-oxo-2H-pyran-2-yl)ethyl]-1-naphthalenyl ester, [1 S-[1 α,3 α,7β,8β(2S*,4S*),8aβ]]-), rosuvastatin calcium (6-Heptenoic acid, 7-(4-(4-fluorophenyl)-6-(1-methylethyl)-2-(methyl(methylsulfonyl)amino)-5-pyrimd inyl)-3,5-dihydroxy- calcium salt (2:1) (S-(R*, S*-(E))) ), meglutol (2-hydroxy-2-methyl-1,3-propandicarboxylic acid), lovastatin (Butanoic acid, 2-methyl-, 1,2,3,7,8,8a-hexahydro-3,7-dimethyl-8-[2-(tetrahydro-4-hydroxy-6-oxo-2H-pyran-2-yl)ethyl]-1-naphthalenyl ester, [1S-[1 α(R*),3 α,7β,8β(2S*,4S*),8aβ]]-), or an analogue or derivative thereof.

O. Hydroorotate Dehydrogenase Inhibitors

In one aspect of the present invention, an anastomotic connection device is therapeutically associated with a hydroorotate dehydrogenase inhibitor, where exemplary compounds having this biological activity include: leflunomide (4-Isoxazolecarboxamide, 5-methyl-N-[4-(trifluoromethyl)phenyl]-), laflunimus (2-Propenamide, 2-cyano-3-cyclopropyl-3-hydroxy-N-(3-methyl-4(trifluoromethyl)phenyl)-, (Z)-), or an analogue or derivative thereof.

P. IKK2 Inhibitors

In one aspect of the present invention, an anastomotic connection device is therapeutically associated with an IKK2 inhibitor, where exemplary compounds having this biological activity include: MLN-120B, SPC-839, or an analogue or derivative thereof.

Q. IL-1, ICE & IRAK Antagonists

In one aspect of the present invention, an anastomotic connection device is therapeutically associated with an IL-1, ICE & IRAK antagonist, where exemplary compounds having this biological activity include: E-5090 (2-Propenoic acid, 3-(5-ethyl-4-hydroxy-3-methoxy-1-naphthalenyl)-2-methyl-, (Z)-), CH-164, CH-172, CH-490, AMG-719, iguratimod (N-[3-(Formylamino)-4-oxo-6-phenoxy-4H-chromen-7-yl] methanesulfonamide), AV94-88, pralnacasan (6H-Pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-), (2S-cis)-5-[Benzyloxycarbonylamino-1,2,4,5,6,7-hexahydro-4-(oxoazepino[3,2,1-hi]indole-2-carbonyl)-amino]-4-oxobutanoic acid, AVE-9488, Esonarimod (Benzenebutanoic acid, Alpha-[(acetylthio)methyl]-4-methyl-Gamma-oxo-), pralnacasan (6H-Pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-), tranexamic acid (Cyclohexanecarboxylic acid, 4-(aminomethyl)-, trans-), Win-72052, Romazarit (Ro-31-3948) (Propanoic acid, 2-[[2-(4-chlorophenyl)-4-methyl-5-oxazolyl]methoxy]-2-methyl-), PD-163594, SDZ-224-015 (L-Alaninamide N-((phenylmethoxy)carbonyl)-L-valyl-N-((1S)-3-((2,6-dichlorobenzoyl)oxy)- 1-(2-ethoxy-2-oxoethyl)-2-oxopropyl)-), L-709049 (L-Alaninamide, N-acetyl-L-tyrosyl-L-valyl-N-(2-carboxy-1-formylethyl)-, (S)-), TA-383 (1H-Imidazole, 2-(4-chlorophenyl)-4,5-dihydro-4,5-diphenyl-, monohydrochloride, cis-), EI -1507-1 (6a,12a-Epoxybenz[a]anthracen-1,12(2H,7H)-dione, 3,4-dihydro-3,7-dihydroxy-8-methoxy-3-methyl-), Ethyl 4-(3,4-dimethoxyphenyl)-6,7-dimethoxy-2-(1,2,4-triazol-1-yl methyl)quinoline-3-carboxylate, EI-1 941-1, TJ-114, anakinra (Interleukin 1 receptor antagonist (human isoform x reduced), N2-L-methionyl-), or an analogue or derivative thereof.

R. IL-4 Agonists

In one aspect of the present invention, an anastomotic connection device is therapeutically associated with an IL-4 agonist, where exemplary compounds having this biological activity include: glatiramir acetate (L-Glutamic acid, polymer with L-alanine, L-lysine and L-tyrosine, acetate (salt), or an analogue or derivative thereof.

S. Immunomodulatory Agents

In one aspect of the present invention, an anastomotic connection device is therapeutically associated with an immunomodulatory agent, where exemplary compounds having this biological activity include: Biolimus, ABT-578, methylsulfamic acid 3-(2-methoxyphenoxy)-2-[[(methylamino)sulfonyl]oxy]propyl ester, sirolimus, CCI-779 (Rapamycin 42-(3-hydroxy-2-(hydroxymethyl)-2-methylpropanoate)), LF-15-0195, NPC15669 (L-Leucine, N-[[(2,7-dimethyl-9H-fluoren-9-yl)methoxy]carbonyl]-), NPC-15670 (L-Leucine, N-[[(4,5-dimethyl-9H-fluoren-9-yl)methoxy]carbonyl]-), N PC-16570 (4-[2-(Fluoren-9-yl)ethyloxycarbonyl]aminobenzoic acid), sulfosfamide (Ethanol, 2-[[3-(2-chloroethyl)tetrahydro-2H-1,3,2-oxazaphosphorin-2-yl]amino]-, methanesulfonate (ester), P-oxide ), tresperimus (2-[N-[4-(3-Aminopropylamino)butyl] carbamoyloxy]-N-(6-guanidinohexyl)acetamide), 4-[2-(Fluoren-9-yl)ethoxycarbonylamino]-benzohydroxamic acid, laquinimod, PBI -1411, azathioprine (6-[(1-Methyl-4-nitro-1H-imidazol-5-yl)thio]-1H-purine), PBI0032, beclometasone, MDL-28842 (9H-Purin-6-amine, 9-(5-deoxy-5-fluoro-β-D-threo-pent-4-enofuranosyl)-, (Z)-), FK-788, AVE-1726, ZK-90695, ZK-90695, Ro-54864, didemnin-B, Illinois (Didemnin A, N-[1-(2-hydroxy- 1-oxopropyl)-L-prolyl]-, (S)-), SDZ-62-826 (Ethanaminium, 2-[[hydroxy[[1-[(octadecyloxy)carbonyl]-3-piperidinyl]methoxy]phosphinyl]oxy]-N,N,N-trimethyl-, inner salt ), argyrin B ((4S,7S,13R,22R)-13-Ethyl-4-(1H-indol-3-ylmethyl)-7-(4-methoxy-1H-indol-3-ylmethyl)18,22-dimethyl-16-methyl-ene-24-thia-3,6,9,12,15,18,21,26-octaazabicyclo[21,2,1 ]-hexacosa-1 (25),23(26)-diene-2,5,8,11,14,17,20-heptaone ), everolimus (Rapamycin, 42-O-(2-hydroxyethyl)-), SAR-943, L-687795, 6-[(4-Chlorophenyl)sulfinyl]-2,3-dihydro-2-(4-methoxyphenyl)-5-methyl-3-oxo-4-pyridazinecarbonitrile, 91Y78 (1H-Imidazo[4,5-c]pyridin-4-amine, 1-β-D-ribofuranosyl-), auranofin (Gold, (1-thio-β-D-glucopyranose 2,3,4,6-tetraacetato-S)(triethylphosphine)-), 27-0-Demethylrapamycin, tipredane (Androsta-1,4-dien-3-one, 17-(ethylthio)-9-fluoro-11-hydroxy-17-(methylthio)-, (11β, 17Alpha)-), AI-402, LY-1 78002 (4-Thiazolidinone, 5-[[3,5-bis(1,1-dimethylethyl)-4-hydroxyphenyl]methylene]-), SM-8849 (2-Thiazolamine, 4-[1-(2-fluoro[1,1′-biphenyl]-4-yl)ethyl]-N-methyl-), piceatannol, resveratrol, triamcinolone acetonide (Pregna-1,4-diene-3,20-dione, 9-fluoro-11,21-dihydroxy-16,17-[(1-methylethylidene)bis(oxy)]-, (11β,16Alpha)-), ciclosporin (Cyclosporin A-), tacrolimus (15,19-Epoxy-3H-pyrido(2,1-c)(1,4)oxaazacyclotricosine-1,7,20,21(4H,23H)-tetrone, 5,6,8,11,12,13,14,15,16,17,18,19,24,25,26,26a-hexadecahydro-5,19-dihydroxy-3-(2-(4-hydroxy-3-methoxycyclohexyl)-1-methylethenyl)-14,16-dimethoxy-4,10,12,18-tetramethyl-8-(2-propenyl)-, (3S-(3R*(E(1S*,3S*,4S*)),4S*,5R*,8S*,9E,12R*,14R*,15S*,16R*,18S*,19S*,26aR*))-), gusperimus (Heptanamide, 7-[(aminoiminomethyl)amino]-N-[2-[[4-[(3-aminopropyl)amino]butyl]amino]-1-hydroxy-2-oxoethyl]-, (+/−)-), tixocortol pivalate (Pregn-4-ene-3,20-dione, 21-[(2,2-dimethyl-1-oxopropyl)thio]-11,17-dihydroxy-, (11β)-), alefacept (1-92 LFA-3 (Antigen) (human) fusion protein with immunoglobulin G1 (human hinge-CH2-CH3 Gammal-chain), dimmer), halobetasol propionate (Pregna-1,4-diene-3,20-dione, 21-chloro-6,9-difluoro-11-hydroxy-16-methyl-17-(1-oxopropoxy)-, (6Alpha,11 β,16β)-), iloprost trometamol (Pentanoic acid, 5-[hexahydro-5-hydroxy-4-(3-hydroxy-4-methyl-1-octen-6-ynyl)-2(1H)-pentalenylidene]-), beraprost (1H-Cyclopenta[b]benzofuran-5-butanoic acid, 2,3,3a,8b-tetrahydro-2-hydroxy-1-(3-hydroxy-4-methyl-1-octen-6-ynyl)-), rimexolone (Androsta-1,4-dien-3-one,11-hydroxy-16,17-dimethyl-17-(1-oxopropyl)-, (11β,16Alpha,17β)-), dexamethasone (Pregna-1,4-diene-3,20-dione,9-fluoro-11,17,21-trihydroxy-16-methyl-, (11β,16Alpha)-), sulindac (cis-5-fluoro-2-methyl-1-[(p-methylsulfinyl)benzylidene]indene-3-acetic acid), proglumetacin (1H-Indole-3-acetic acid, 1-(4-chlorobenzoyl)-5-methoxy-2-methyl-, 2-(4-(3-((4-(benzoylamino)-5-(dipropylamino)-1,5-dioxopentyl)oxy)propyl)-1-piperazinyl)ethylester, (+/−)-), alclometasone dipropionate (Pregna-1,4-diene-3,20-dione, 7-chloro-11-hydroxy-16-methyl-17,21-bis(1-oxopropoxy)-, (7Alpha,11β,16Alpha)-), pimecrolimus (15,19-Epoxy-3H-pyrido(2,1-c)(1,4)oxaazacyclotricosine-1,7,20,21(4H ,23H)-tetrone, 3-(2-(4-chloro-3-methoxycyclohexyl)-1-methyletheny)-8-ethyl-5,6,8,11,12,13,14,15,16,17,18,19,24,25,26,26a-hexadecahydro-5,19-dihydroxy-14,16-dimethoxy-4,10,12,18-tetramethyl-, (3S-(3R*(E(1S*,3S*,4R*)),4S*,5R*,8S*,9E,12R*,14R*,15S*,16R*,18S*,19S*,26aR*))-), hydrocortisone-17-butyrate (Pregn-4-ene-3,20-dione, 11,21-dihydroxy-17-(1-oxobutoxy)-, (11β)-), mitoxantrone (9,10-Anthracenedione, 1,4-dihydroxy-5,8-bis[[2-[(2-hydroxyethyl)amino]ethyl]amino]-), mizoribine (1H-Imidazole-4-carboxamide, 5-hydroxy-1-β-D-ribofuranosyl-), prednicarbate (Pregna-1,4-diene-3,20-dione, 17-[(ethoxycarbonyl)oxy]-11-hydroxy-21-(1-oxopropoxy)-, (11β)-), lobenzarit (Benzoic acid, 2-[(2-carboxyphenyl)amino]-4-chloro-), glucametacin (D-Glucose, 2-[[[1-(4-chlorobenzoyl)-5-methoxy-2-methyl-1H-indol-3-yl]acetyl]amino]-2-deoxy-), fluocortolone monohydrate ((6Alpha)-fluoro-16Alpha-methylpregna-1,4-dien-11β,21-diol-3,20-dione), fluocortin butyl (Pregna-1,4-dien-21-oic acid, 6-fluoro-11-hydroxy-16-methyl-3,20-dioxo-, butyl ester, (6Alpha,11β,16AIpha)-), difluprednate (Pregna-1,4-diene-3,20-dione, 21-(acetyloxy)-6,9-difluoro-11-hydroxy-17-(1-oxobutoxy)-, (6Alpha,11β)-), diflorasone diacetate (Pregna-1,4-diene-3,20-dione, 17,21-bis(acetyloxy)-6,9-difluoro-11-hydroxy-16-methyl-, (6Alpha,11β,16β)-), dexamethasone valerate (Pregna-1,4-diene-3,20-dione, 9-fluoro-11,21-dihydroxy-16-methyl-17-[(1-oxopentyl)oxy]-, (11β,16α)-), methylprednisolone, deprodone propionate (Pregna-1,4-diene-3,20-dione, 11-hydroxy-17-(1-oxopropoxy)-, (11.beta.)-), bucillamine (L-Cysteine, N-(2-mercapto-2-methyl-1-oxopropyl)-), amcinonide (Benzeneacetic acid, 2-amino-3-benzoyl-, monosodium salt, monohydrate ), acemetacin (1H-Indole-3-acetic acid, 1-(4-chlorobenzoyl)-5-methoxy-2-methyl-, carboxymethyl ester) , or an analogue or derivative thereof. Further, analogues of rapamycin include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives can be found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

The structures of sirolimus, everolimus, and tacrolimus are provided below:

| Name | Code Name | Company | Structure |
|---|---|---|---|
| Everolimus | SAR-943 | Novartis | See below |
| Sirolimus | AY-22989 | Wyeth | See below |
| Rapamune | NSC-226080 | | |
| Rapamycin | | | |
| Tacrolimus | FK506 | Fujisawa | See below |

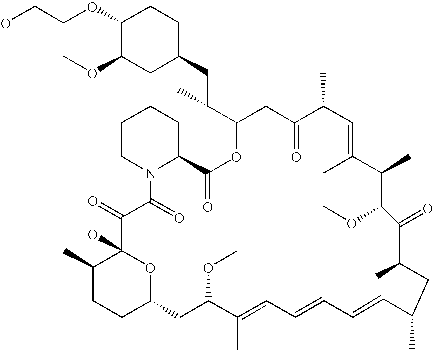

Everolimus

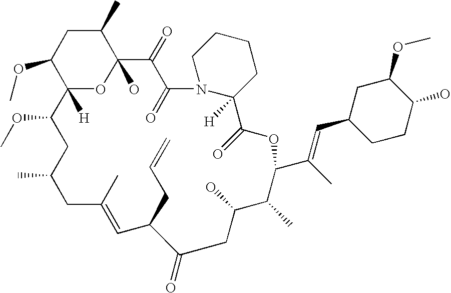

Tacrolimus

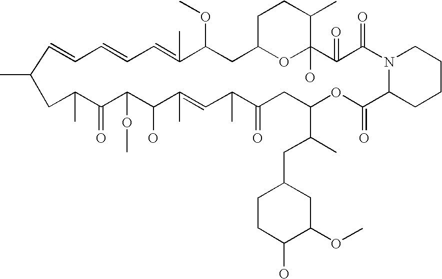

Sirolimus

Further sirolimus analogues and derivatives include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives include ABT-578 and others may be found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO93/11130, WO 93/10122, WO 93/04680, WO 92/14737, and WO 92/05179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241, 5,200,411; 5,198,421; 5,147,877; 5,140,018; 5,116,756; 5,109,112; 5,093,338; and 5,091,389.

T. Inosine Monophosphate Dehydrogenase Inhibitors

In one aspect of the present invention, an anastomotic connection device is therapeutically associated with an inosine monophosphate dehydrogenase inhibitor, where exemplary compounds having this biological activity include: Mycophenolate Mofetil (4-Hexenoic acid, 6-(1,3-dihydro-4-hydroxy-6-methoxy-7-methyl-3-oxo-5-isobenzofuranyl)-4-methyl-, 2-(4-morpholinyl)ethyl ester, (E)-), ribavirin (1H-1,2,4-Triazole-3-carboxamide, 1-β-D-ribofuranosyl-), tiazofurin (4-Thiazolecarboxamide, 2-β-D-ribofuranosyl-), viramidine, aminothiadiazole, thiophenfurin, tiazofurin, or an analogue or derivative thereof. Additional representative examples are included in U.S. Patent and Patent Application Publication Nos. U.S. Pat. No. 5,536,747, U.S. Pat. No. 5,807,876, U.S. Pat. No. 5,932,600, U.S. Pat. No. 6,054,472, U.S. Pat. No. 6,128,582, U.S. Pat. No. 6,344,465, U.S. Pat. No. 6,395,763, U.S. Pat. No. 6,399,773, U.S. Pat. No. 6,420,403, U.S. Pat. No. 6,479,628, U.S. Pat. No. 6,498,178, U.S. Pat. No. 6,514,979, U.S. Pat. No. 6,518,291, U.S. Pat. No. 6,541,496, U.S. Pat. No. 6,596,747, U.S. Pat. No. 6,617,323, U.S. Pat. No. 6,624,184, 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, 2003/0195202A1, and PCT Publication Nos. WO0024725A1, WO0025780A1, WO 0026197A1, WO0051615A1, WO0056331A1, WO0073288A1, WO0100622A1, WO 0166706A1, WO0179246A2, WO0181340A2, WO01 85952A2, WO0216382A1, WO0218369A2, WO2051814A1, WO2057287A2, WO2057425A2, WO2060875A1, WO2060896A1, WO2060898A1, WO2068058A2, WO3020298A1, WO3037349A1, WO3039548A1, WO3045901A2, WO3047512A2, WO3053958A1, WO3055447A2, WO3059269A2, WO3063573A2, WO3087071A1, WO9001545A1, WO9740028A1, WO9741211A1, WO9840381A1, and WO 9955663A1.

U. Leukotriene Inhibitors

In one aspect of the present invention, an anastomotic connection device is therapeutically associated with a leukotreine inhibitor, where exemplary compounds having this biological activity include: ONO-4057(Benzenepropanoic acid, 2-(4-carboxybutoxy)-6-[[6-(4-methoxyphenyl)-5-hexenyl]oxy]-, (E)-), ONO-LB-448, pirodomast 1,8-Naphthyridin-2(1H )-one, 4-hyd roxy-1-phenyl-3-(1-pyrrolidinyl)-[CAS], Sch-40120 (Benzo[b][1,8]naphthyridin-5(7H)-one, 10-(3-chlorophenyl)-6,8,9,10-tetrahydro-), L-656224 (4-Benzofuranol, 7-chloro-2-[(4-methoxyphenyl)methyl]-3-methyl-5-propyl-), MAFP (methyl arachidonyl fluorophosphonate), ontazolast (2-Benzoxazolamine, N-[2-cyclohexyl-1-(2-pyridinyl)ethyl]-5-methyl-, (S)-), amelubant (Carbamic acid, ((4-((3-((4-(1-(4-hydroxyphenyl)-1-methylethyl )phenoxy)methyl)phenyl) methoxy)phenyl)iminomethyl)-ethyl ester), SB-201993 (Benzoic acid, 3-[[[[6-[(1E)-2-carboxyethenyl]-5-[[8-(4-methoxyphenyl)octyl]oxy]-2-pyridinyl]methyl]thio]methyl]-), LY-203647 (Ethanone,1-[2-hydroxy-3-propyl-4-[4-[2-[4-(1H-tetrazol-5-yl)butyl]-2H-tetrazol-5-yl]butoxy]phenyl]-), LY-210073, LY-223982 (Benzenepropanoic acid, 5-(3-carboxybenzoyl)-2-[[6-(4-methoxyphenyl)-5-hexenyl]oxy]-, (E)-), LY-293111 (Benzoic acid, 2-[3-[3-[(5-ethyl-4′-fluoro-2-hydroxy[1,1′-biphenyl]-4-yl)oxy]propoxy]-2-propylphenoxy]-), SM-9064 (Pyrrolidine, 1-[4,11-dihydroxy-13-(4-methoxyphenyl)-1-oxo-5,7,9-tridecatrienyl]-, (E,E,E)-), T-0757 (2,6-Octadienamide, N-(4-hydroxy-3,5-dimethylphenyl)-3,7-dimethyl-, (2 E)-), or an analogue or derivative thereof.

V. MCP-1 Antagonists

In one aspect of the present invention, an anastomotic connection device is therapeutically associated with a MCP-1 antagonist, where exemplary compounds having this biological activity include: nitronaproxen (2-Napthaleneacetic acid, 6-methoxy-Alpha-methyl 4-(nitrooxy)butyl ester (AlphaS)-), Bindarit (2-(1-benzylindazol-3-ylmethoxy)-2-methylpropanoic acid), 1-alpha-25 dihydroxy vitamin D₃, or an analogue or derivative thereof.

W. MMP Inhibitors

In one aspect of the present invention, an anastomotic connection device is therapeutically associated with a MMP inhibitor, where exemplary compounds having this biological activity include: D-9120, doxycycline (2-Naphthacenecarboxamide, 4-(dimethylamino)-1,4,4a,5,5a,6,11,12a-octahydro-3,5,10,12,12a-pentahydroxy-6-methyl-1,11-dioxo- [4S-(4α,4aα,5α,5aα,6α,12aα)]-), BB-2827, BB-1101 (2S-allyl-N1-hydroxy-3R-isobutyl-N4-(1S-methylcarbamoyl-2-phenylethyl)-succinamide), BB-2983, solimastat (N′-[2,2-Dimethyl-1(S)-[N-(2-pyridyl)carbamoyl]propyl]-N4-hydroxy-2(R)-isobutyl-3(S)-methoxysuccinamide), Batimastat (Butaned iamide, N4-hydroxy-N1-[2-(methylamino)-2-oxo-1-(phenylmethyl)ethyl]-2-(2-methylpropyl)-3-[(2-thienylthio)methyl]-, [2R-[1(S*),2R*,3S*]]-), CH-138, CH-5902, D-1927, D-5410, EF-13 (y-linolenic acid lithium salt),CMT-3 (2-Naphthacenecarboxamide, 1,4,4a,5,5a,6,11,12a-octahydro-3,10,12,12a-tetrahydroxy-1,11-dioxo-, (4aS,5aR,12aS)-), Marimastat (N-[2,2-Dimethyl-1(S)-(N-methylcarbamoyl)propyl]-N,3(S)-dihydroxy-2(R)-isobutylsuccinamide), TIMP'S,ONO-4817, rebimastat (L-Valinamide, N-((2S)-2-mercapto-1-oxo-4-(3,4,4-trimethyl-2,5-dioxo-1-imidazolidinyl)butyl)-L-leucyl-N,3-dimethyl-), PS-508, CH-715, nimesulide (Methanesulfonamide, N-(4-nitro-2-phenoxyphenyl)-), hexahydro-2-[2(R)-[1(RS)-(hydroxycarbamoyl)-4-phenylbutyl]nonanoyl]-N-(2,2,6,6-etramethyl-4-piperidinyl)-3(S)-pyridazine carboxamide, Rs-113-080, Ro-1130830, Cipemastat (1-Piperidinebutanamide, β-(cyclopentylmethyl)-N-hydroxy-y-oxo-Alpha-[(3,4,4-trimethyl-2,5-dioxo-1-imidazolidinyl)methyl]-,(AlphaR,βR)-), 5-(4′-biphenyl)-5-[N-(4-nitrophenyl)piperazinyl]barbituric acid, 6-methoxy-1,2,3,4-tetrahydro-norharman-1-carboxylic acid, Ro-31-4724 (L-Alanine, N-[2-[2-(hydroxyamino)-2-oxoethyl]-4-methyl-1-oxopentyl]-L-leucyl-, ethyl ester), prinomastat (3-Thiomorpholinecarboxamide, N-hydroxy-2,2-dimethyl-4-((4-(4-pyridinyloxy) phenyl)sulfonyl)-, (3R)-), AG-3433 (1H-Pyrrole-3-propanic acid, 1-(4′-cyano[1,1′-biphenyl]-4-yl)-b-[[[(3S )-tetrahydro-4,4-dimethyl-2-oxo-3-furanyl]amino]carbonyl]-, phenylmethyl ester, (bS)-), PNU-142769 (2H-Isoindole-2-butanamide, 1,3-dihydro-N-hydroxy-Alpha-[(3S)-3-(2-methylpropyl)-2-oxo-1-(2-phenylethyl)-3-pyrrolidinyl]-1,3-dioxo-, (AlphaR)-), (S)-1-[2-[[[(4,5-Dihydro-5-thioxo-1,3,4-thiadiazol-2-yl)amino]-carbonyl]amino]-1-oxo-3-(pentafluorophenyl)propyl]-4-(2-pyridinyl)piperazine, SU-5402 (1H-Pyrrole-3-propanoic acid, 2-[(1,2-dihydro-2-oxo-3H-indol-3-ylidene)methyl]-4-methyl-), SC-77964, PNU-171829, CGS-27023A, N-hydroxy-2(R)-[(4-methoxybenzene-sulfonyl)(4-picolyl)amino]-2-(2-tetrahydrofuranyl)-acetamide, L-758354 ((1,1′-Biphenyl)-4-hexanoic acid, Alpha-butyl-Gamma-(((2,2-dimethyl-1-((methylamino)carbonyl)propyl)amino)carbonyl)-4′-fluoro-, (αS-(αR*,GammaS*(R*)))-), GI-155704A, CPA-926 or an analogue or derivative thereof. Additional representative examples are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,691,381; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359.

X. NF Kappa B Inhibitors

In one aspect of the present invention, an anastomotic connection device is therapeutically associated with a NF kappa B inhibitor, where exemplary compounds having this biological activity include: AVE-0545, Oxi-104 (Benzamide, 4-amino-3-chloro-N-(2-(diethylamino)ethyl)-), dexlipotam, INDRA, R-flurbiprofen ([1,1′-Biphenyl]-4-acetic acid, 2-fluoro-Alpha-methyl), SP100030 (2-chloro-N-[3,5-di(trifluoromethyl)phenyl]-4-(trifluoromethyl)pyrimidine-5-carboxamide), AVE-0545, Viatris, AVE-0547, Bay 11-7082, Bay 11-7085, 15 deoxy-prostaylandin J2, bortezomib (Boronic acid, [(1R)-3-methyl-1-[[(2S)-1-oxo-3-phenyl-2-[(pyrazinylcarbonyl)amino]propyl]amino]butyl]-, or an analogue or derivative thereof.

Y. NO Agonists

In one aspect of the present invention, an anastomotic connection device is therapeutically associated with a NO antagonist, where exemplary compounds having this biological activity include: NCX-4016 (Benzoic acid, 2-(acetyloxy)-, 3-((nitrooxy)methyl)phenyl ester), NCX-2216, L-arginine, or an analogue or derivative thereof.

Z. P38 MAP Kinase Inhibitors

In one aspect of the present invention, an anastomotic connection device is therapeutically associated with a P38 MAP kinase inhibitor, where exemplary compounds having this biological activity include: GW-2286, CGP-52411, BIRB-798, SB220025, RO-320-1195, RWJ-67657, RWJ-68354, SCIO-469, SCIO-323, AMG-548, CMC-146, SD-31145, CC-8866, Ro-320-1195, PD-98059 (4H-1-Benzopyran-4-one, 2-(2-amino-3-methoxyphenyl)-), CGH-2466, doramapimod, SB-203580 (Pyridine, 4-[5-(4-fluorophenyl)-2-[4-(methylsulfinyl)phenyl]-1H-imidazol-4-yl]-), SB-220025 ((5-(2-Amino-4-pyrimidinyl)-4-(4-fluorophenyl)-1-(4-piperidinyl)imidazole)), SB-281832, PD169316, SB202190 or an analogue or derivative thereof. Additional representative examples are included in U.S. Patent Nos., U.S. Patent Application and PCT Publication Nos. U.S. Pat. No. 6,300,347; U.S. Pat. No. 6,316,464; U.S. Pat. No. 6,316,466; U.S. Pat. No. 6,376,527; U.S. Pat. No. 6,444,696; U.S. Pat. No. 6,479,507; U.S. Pat. No. 6,509,361; U.S. Pat. No. 6,579,874; U.S. Pat. No. 6,630,485; 2001/0044538A1; US2002/0013354A1; US2002/0049220A1; 2002/0103245A1; 2002/0151491A1; 2002/0156114A1; 2003/0018051A1; 2003/0073832A1; 2003/0130257A1; 2003/0130273A1; 2003/0130319A1; 2003/0139388A1; 2003/0139462A1; 2003/0149031A1; 2003/0166647A1; 2003/0181411A1; WO0063204A2; WO0121591A1; WO0135959A1; WO0174811 A2; WO0218379A2; WO2064594A2; WO2083622A2; WO2094842A2; WO2096426A1; WO2101015A2; WO2103000A2; WO3008413A1; WO3016248A2; WO3020715A1; WO3024899A2; WO3031431A1; WO3040103A1; WO3053940A1; WO3053941A2; WO3063799A2; WO3079986A2; WO3080024A2; WO3082287A1; WO9744467A1; WO9901449A1; and WO9958523A1.

AA. Phosphodiesterase Inhibitors

In one aspect of the present invention, an anastomotic connection device is therapeutically associated with a phosphodiesterase inhibitor, where exemplary compounds having this biological activity include: CDP-840 (Pyridine, 4-[(2R)-2-[3-(cyclopentyloxy)-4-methoxyphenyl]-2-phenylethyl]-), CH-3697, CT-2820, D-22888 (Imidazo[1,5-a]pyrido[3,2-e]pyrazin-6(5H)-one, 9-ethyl-2-methoxy-7-methyl-5-propyl-), D-4418 (8-Methoxyquinoline-5-[N-(2,5-dichloropyridin-3-yl)]carboxamide),1-(3-cyclopentyloxy-4-methoxyphenyl)-2-(2,6-dichloro-4-pyridyl) ethanone oxime, D-4396, ONO-6126, CDC-998, CDC-801, V-11294A (3-[3-(Cyclopentyloxy)-4-methoxybenzyl]-6-(ethylamino)-8-isopropyl-3H-purine hydrochloride), S,S′-methylene-bis(2-(8-cyclopropyl-3-propyl-6-(4-pyridylmethylamino)-2-thio-3H-purine)) tetrahydrochloride, Rolipram (2-Pyrrolidinone, 4-[3-(cyclopentyloxy)-4-methoxyphenyl]-), CP-293121, CP-353164 (5-(3-Cyclopentyloxy-4-methoxyphenyl)pyridine-2-carboxamide), oxagrelate (6-Phthalazinecarboxylic acid, 3,4-dihydro-1-(hydroxymethyl)-5,7-dimethyl-4-oxo-, ethyl ester ), PD-1 68787, ibudilast (1-Propanone, 2-methyl-1-[2-(1-methylethyl)pyrazolo[1,5-a]pyridin-3-yl]-), oxagrelate (6-Phthalazinecarboxylic acid, 3,4-dihydro-1-(hydroxymethyl)-5,7-dimethyl-4-oxo-, ethyl ester ), griseolic acid (Alpha-L-talo-Oct-4-enofuranuronic acid,1-(6-amino-9H-purin-9-yl)-3,6-anhydro-6-C-carboxy-1,5-dideoxy-), KW-4490, KS-506, T-440, roflumilast (Benzamide, 3-(cyclopropylmethoxy)-N-(3,5-dichloro-4-pyridinyl)-4-(difluoromethoxy)-), rolipram, milrinone, triflusinal (Benzoic acid, 2-(acetyloxy)-4-(trifluoromethyl)-), anagrelide hydrochloride (Imidazo[2,1-b]quinazolin-2(3H)-one, 6,7-dichloro-1,5-dihydro-, monohydrochloride), cilostazol (2(1H)-Quinolinone, 6-[4-(1-cyclohexyl-1H-tetrazol-5-yl)butoxy]-3,4-dihydro-), propentofylline (1H-Purine-2,6-dione, 3,7-dihydro-3-methyl-1-(5-oxohexyl)-7-propyl-), sildenafil citrate (Piperazine,1-((3-(4,7-dihydro-1-methyl-7-oxo-3- propyl-1H-pyrazolo(4,3-d)pyrimidin-5-yl)-4-ethoxyphenyl)sulfonyl)-4-methyl, 2-hydroxy-1,2,3-propanetricarboxylate- (1:1) ), tadalafil (Pyrazino(1′,2′:1,6)pyrido(3,4-b)indole1,4-dione, 6-(1,3-benzodioxol-5-yl)-2,3,6,7,12,12a-hexahydro-2-methyl-, (6R-trans) ), vardenafil (Piperazine, 1-(3-(1,4-dihydro-5-methyl(-4-oxo-7-propylimidazo(5,1-f)(1,2,4)-triazin-2-yl)-4-ethoxyphenyl)sulfonyl)-4-ethyl-), milrinone ([3,4′-Bipyridine]-5-carbonitrile, 1,6-dihydro-2-methyl-6-oxo-), enoximone (2H-Imidazol-2-one, 1,3-dihydro-4-methyl-5-[4-(methylthio)benzoyl]-), theophylline (1H-Purine-2,6-dione, 3,7-dihydro-1,3-dimethyl-), ibudilast (1-Propanone, 2-methyl-1-[2-(1-methylethyl)pyrazolo[1,5-a]pyridin-3-yl]-), aminophylline (1H-Purine-2,6-dione, 3,7-dihydro-1,3-dimethyl-, compd. with 1,2-ethanediamine (2:1)-), acebrophylline (7H-Purine-7-acetic acid, 1,2,3,6-tetrahydro-1,3-dimethyl-2,6-dioxo-, compd. with trans-4-[[(2-amino-3,5-dibromophenyl)methyl]amino]cyclohexanol (1:1) ), plafibride (Propanamide, 2-(4-chlorophenoxy)-2-methyl-N-[[(4-morpholinylmethyl)amino]carbonyl]-), loprinone hydrochloride (3-Pyridinecarbonitrile, 1,2-dihydro-5-imidazo[1,2-a]pyridin-6-yl-6-methyl-2-oxo-, monohydrochloride-), fosfosal (Benzoic acid, 2-(phosphonooxy)-), amrinone ([3,4′-Bipyridin]-6(1H)-one, 5-amino-, or an analogue or derivative thereof.

BB. TGF Beta Inhibitors

In one aspect of the present invention, an anastomotic connection device is therapeutically associated with a TGF beta Inhibitor, where exemplary compounds having this biological activity include: mannose-6-phosphate, LF-984, tamoxifen (Etha namine, 2-[4-(1,2-diphenyl-1-butenyl)phenoxy]-N,N-dimethyl-, (Z)-), tranilast, or an analogue or derivative thereof.

CC. Thromboxane A2 Antagonists

In one aspect of the present invention, an anastomotic connection device is therapeutically associated with a thromboxane A2 antagonist, where exemplary compounds having this biological activity include: CGS-22652 (3-Pyridineheptanoic acid, gamma .-[4-[[(4-chlorophenyl)sulfonyl]amino]butyl]-, (.+−. )-), ozagrel (2-Propenoic acid, 3-[4-(1H-imidazol-1-ylmethyl)phenyl]-, (E)-), argatroban (2-Piperidinecarboxylic acid, 1-[5-[(aminoiminomethyl)amino]-1-oxo-2-[[(1,2,3,4-tetrahydro-3-methyl-8-quinolinyl)sulfonyl]amino]pentyl]-4-methyl-), ramatroban (9H-Carbazole-9-propanoic acid, 3-[[(4-fluorophenyl)sulfonyl]amino]-1,2,3,4-tetrahydro-, (R)-), torasemide (3-Pyridinesulfonamide, N-[[(1-methylethyl)amino]carbonyl]-4-[(3-methylphenyl)amino]-), gamma linoleic acid ((Z,Z,Z)-6,9,12-Octadecatrienoic acid ), seratrodast (Benzeneheptanoic acid, zeta-(2,4,5-trimethyl-3,6-dioxo-1,4-cyclohexadien-1-yl)-, (+/−)-, or an analogue or derivative thereof.

DD. TNFa Antagonists/TACE Inhibitors

In one aspect of the present invention, an anastomotic connection device is therapeutically associated with a TNFa Antagonist/TACE Inhibitor, where exemplary compounds having this biological activity include: E-5531 (2-Deoxy-6-0-[2-deoxy-3-0-[3(R)-[5(Z)-dodecenoyloxy]-decyl]-6-0-methyl-2-(3-oxotetradecanamido)-4-O-phosphono-β-D-glucopyranosyl]-3-0-[3(R)-hydroxydecyl]-2-(3-oxotetradecanamido)-Alpha-D-glucopyranose-1-O-phosphate), AZD-4717, glycophosphopeptical, UR-12715 (Benzoic acid, 2-hydroxy-5-[[4-[3-[4-(2-methyl-1H-imidazol[4,5-c]pyridin-1-yl]methyl]-1-piperid inyl]-3-oxo-1-phenyl-1-propenyl]phenyl]azo] (Z) ), PMS-601, AM-87, xyloadenosine (9H-Purin-6-amine, 9-β-D-xylofuranosyl-), RDP-58, RDP-59, BB2275, benzydamine, E-3330 (Undecanoic acid, 2-[(4,5-dimethoxy-2-methyl-3,6-dioxo-1,4-cyclohexadien-1-yl)methylene]-, (E)-), N-[D, L-2-(hydroxyaminocarbonyl)methyl-4-methylpentanoyl]-L-3-(2′-naphthyl)alanyl-L-alanine, 2-aminoethyl amide, CP-564959, MLN-608, SPC-839, ENMD-0997, Sch-23863 ((2-[10,11-Dihydro-5-ethoxy-5H-dibenzo [a,d] cyclohepten-S-yl]-N,N-dimethyl-ethanamine), SH-636, PKF-241-466, PKF-242-484, TNF-484A, cilomilast (Cis-4-cyano-4-[3-(cyclopentyloxy)-4-methoxyphenyl]cyclohexane-1-carboxylic acid), GW-3333, GW-4459, BMS-561392, AM-87, cloricromene (Acetic acid, [[8-chloro-3-[2-(diethylamino)ethyl]-4-methyl-2-oxo-2H-1-benzopyran-7-yl]oxy]-, ethyl ester), thalidomide (1H-Isoindole-1,3(2H)-dione, 2-(2,6-dioxo-3-piperidinyl)-), vesnarinone (Piperazine, 1-(3,4-dimethoxybenzoyl)-4-(1,2,3,4-tetrahydro-2-oxo-6-quinolinyl)-), infliximab, lentinan, etanercept (1-235-Tumor necrosis factor receptor (human) fusion protein with 236-467-immunoglobulin G1 (human gammal-chain Fc fragment) ), diacerein (2-Anthracenecarboxylic acid, 4,5-bis(acetyloxy)-9,10-dihydro-9,10-dioxo-, or an analogue or derivative thereof.

EE. Tyrosine Kinase Inhibitors

In one aspect of the present invention, an anastomotic connection device is therapeutically associated with a tyrosine kinase inhibitor, where exemplary compounds having this biological activity include: SKI-606, ER-068224, SD-208, N-(6-Benzothiazolyl)-4-(2-(1-piperazinyl)pyrid-5-yl)-2-pyrimidineamine, celastrol (24,25,26-Trinoroleana-1(10),3,5,7-tetraen-29-oic acid, 3-hydroxy-9,13-dimethyl-2-oxo-, (9.beta.,13Alpha,14β,20Alpha)-), CP-127374 (Geldanamycin, 17-demethoxy-17-(2-propenylamino)-), CP-564959, PD-171026, CGP-52411(1H-Isoindole-1,3(2H)-dione, 4,5-bis(phenylamino)-), CGP-53716 (Benzamide, N-[4-methyl-3-[[4-(3-pyridinyl)-2-pyrimidinyl]amino]phenyl]-), imatinib (4-((Methyl-1-piperazinyl)methyl)-N-[4-methyl-3-[[4-(3-pyridinyl)-2-pyrimidinyl]amino]-phenyl]benzamide methanesulfonate), NVP-MK980-NX, KF-250706 (13-Chloro,5(R),6(S)-epoxy-14,16-dihydroxy-11-(hydroyimino)-3(R)-methyl-3,4,5,6,11,12-hexahydro-1H-2-benzoxacyclotetradecin-1-one), 5-[3-[3-methoxy-4-[2-[(E)-2-phenylethenyl]-4-oxazolylmethoxy]phenyl]propyl]-3-[2-[(E)-2-phenylethenyl]-4-oxazolylmethyl]-2,4-oxazolidined ione, genistein, or an analogue or derivative thereof.

FF. Vitronectin Inhibitors

In one aspect of the present invention, an anastomotic connection device is therapeutically associated with a vitronectin inhibitor, where exemplary compounds having this biological activity include: O-[9,10-dimethoxy-1,2,3,4,5,6-hexahydro-4-[(1,4,5,6-tetrahydro-2-pyrimidinyl)hydrazono]-8-benz(e)azulenyl]-N-[(phenylmethoxy)carbonyl]-DL-homoserine 2,3-dihydroxypropyl ester, (2S)-Benzoylcarbonylamino-3-[2-((4S)-(3-(4,5-dihydro-1H-imidazol-2-ylamino)propyl)-2,5-dioxo-imidazolidin-1-yl)-acetylamino]-propionate, Sch-221153, S-836, SC-68448 (β-[[2-2-[[[3-[(aminoiminomethyl)amino]phenyl]carbonyl]amino]acetyl]amino]-3,5-dichlorobenzenepropanoic acid), SD-7784, S-247, or an analogue or derivative thereof.

GG. Fibroblast Growth Factor Inhibitors

In one aspect of the present invention, an anastomotic connection device is therapeutically associated with a fibroblast growth factor inhibitor, where exemplary compounds having this biological activity include: CT-052923 ([(2H-benzo[d]1,3-dioxalan-5-methyl)amino][4-(6,7-dimethoxyquinazolin-4-yl)piperazinyl]methane-1-thione, or an analogue or derivative thereof.

HH. Protein Kinase Inhibitors

In one aspect of the present invention, an anastomotic connection device is therapeutically associated with a protein kinase inhibitor, where exemplary compounds having this biological activity include: KP-0201448, NPC 15437 (Hexanamide, 2,6-diamino-N-[[1-(1-oxotridecyl)-2-piperidinyl]methyl]-), fasudil (1H-1,4-Diazepine, hexahydro-1-(5-isoquinolinylsulfonyl)-), midostaurin (Benzamide, N-(2,3,10,11,12,13-hexahydro-10-methoxy-9-methyl-1-oxo-9,13-epoxy-1H,9H-diindolo[1,2,3-gh:3′,2′,1′-Im]pyrrolo[3,4-j][1,7]benzodiazonin-11-yl)-N-methyl-, (9Alpha,10β,11β,13Alpha)-),fasudil (1H-1,4-Diazepine, hexahydro-1-(5-isoquinolinylsulfonyl)-, or an analogue or derivative thereof.

II. PDGF Receptor Kinase Inhibitors

In one aspect of the present invention, an anastomotic connection device is therapeutically associated with a PDGF receptor kinase inhibitor, where exemplary compounds having this biological activity include: RPR-127963E and analogues and derivatives thereof.

JJ. Endothelial Growth Factor Receptor Kinase Inhibitors

In one aspect of the present invention, an anastomotic connection device is therapeutically associated with an endothelial growth factor receptor kinase inhibitor, where exemplary compounds having this biological activity include: CEP-7055, SU-0879 ((E)-3-(3,5-di-tert-Butyl-4-hydroxyphenyl)-2-(aminothiocarbonyl)acrylonitrile), BIBF-1000, or an analogue or derivative thereof.

KK. Retinoic Acid Receptor Antagonists

In one aspect of the present invention, an anastomotic connection device is therapeutically associated with a retinoic acid receptor antagonist, where exemplary compounds having this biological activity include: etarotene (Ro-15-1570) (Naphthalene, 6-[2-[4-(ethylsulfonyl)phenyl]-1-methylethenyl]-1,2,3,4-tetrahydro-1,1,4,4-tetramethyl-, (E)-), (2E,4E)-3-Methyl-5-(2-((E)-2-(2,6,6-trimethyl-1-cyclohexen-1-yl)ethenyl)-1-cyclohexen-1-yl)-2,4-pentadienoic acid, tocoretinate (Retinoic acid, 3,4-dihydro-2,5,7,8-tetramethyl-2-(4,8,12-trimethyltridecyl)-2H-1-benzopyran-6-yl ester, [2R*(4R*,8R*)]-(±)-), aliretinoin (Retinoic acid, cis-9, trans-13-), bexarotene (Benzoic acid, 4-(1-(5,6,7,8-tetrahydro-3,5,5,8,8-pentamethyl-2-naphthalenyl)ethenyl)-, or an analogue or derivative thereof.

LL. Platelet Derived Growth Factor Receptor Kinase Inhibitors

In one aspect of the present invention, an anastomotic connection device is therapeutically associated with a platelet derived growth factor receptor kinase inhibitor, where exemplary compounds having this biological activity include: leflunomide (4-lsoxazolecarboxamide, 5-methyl-N-[4-(trifluoromethyl)phenyl]-, or an analogue or derivative thereof.

MM. Fibrinogin Antagonists

In one aspect of the present invention, an anastomotic connection device is therapeutically associated with a fibrinogin antagonist, where exemplary compounds having this biological activity include: picotamide (1,3-Benzenedicarboxamide, 4-methoxy-N,N′-bis(3-pyridinylmethyl)-, or an analogue or derivative thereof.

NN. Antimycotic Agents

In one aspect of the present invention, an anastomotic connection device is therapeutically associated with an antimycotic agent, where exemplary compounds having this biological activity include: miconazole, sulconizole, parthenolide, rosconitine, nystatin, isoconazole, fluconazole, ketoconasole, imidazole, itraconazole, terpinafine, elonazole, bifonazole, clotrimazole, conazole, terconazole (Piperazine, 1-[4-[[2-(2,4-dichlorophenyl)-2-(1H-1,2,4-triazol-1-ylmethyl)-1,3-dioxolan-4-yl]methoxy]phenyl]-4-(1-methylethyl)-, cis-), isoconazole (1-[2-(2-6-dichlorobenzyloxy)-2-(2-,4-dichlorophenyl)ethyl]), griseofulvin (Spiro[benzofuran-2(3H),1′-[2]cyclohexane]-3,4′-dione, 7-chloro-2′,4,6-trimeth-oxy-6′methyl-, (1′S-trans)-), bifonazole (1H-lmidazole, 1-([1,1′-biphenyl]-4-ylphenylmethyl)-), econazole nitrate (1-[2-[(4-chlorophenyl)methoxy]-2-(2,4-dichlorophenyl)ethyl]-1H-imidazole nitrate), croconazole (1H-Imidazole, 1-[1-[2-[(3-chlorophenyl)methoxy]phenyl]ethenyl]-), sertaconazole (1H-Imidazole, 1-[2-[(7-chlorobenzo[b]thien-3-yl)methoxy]-2-(2,4-dichlorophenyl)ethyl]-), omoconazole (1H-Imidazole, 1-[2-[2-(4-chlorophenoxy)ethoxy]-2-(2 ,4-dichlorophenyl)-1-methylethenyl]-, (Z)-), flutrimazole (1H-Imidazole, 1-[(2-fluorophenyl)(4-fluorophenyl)phenylmethyl]-), fluconazole (1H-1,2,4-Triazole-1-ethanol, Alpha-(2,4-difluorophenyl)-Alpha-(1H-1,2,4-triazol-1-ylmethyl)-), neticonazole (1H-Imidazole, 1-[2-(methylthio)-1-[2-(pentyloxy)phenyl]ethenyl]-, monohydrochloride, (E)-), butoconazole (1H-Imidazole, 1-[4-(4- chlorophenyl)-2-[(2,6-dichlorophenyl)thio]butyl]-, (+/−)-), clotrimazole (1-[(2-chlorophenyl)diphenylmethyl]- 1H-imidazole, or an analogue or derivative thereof.

OO. Bisphosphonates

In one aspect of the present invention, an anastomotic connection device is therapeutically associated with a bisphosphonate, where exemplary compounds of this class include: Clodronate, Alendronate, pamidronate, zoledronate, or an analogue or derivative thereof.

PP. Phospholipase A1 Inhibitors

In one aspect of the present invention, an anastomotic connection device is therapeutically associated with a phospholipase A1 inhibitor, where exemplary compounds having this biological activity include: loteprednol etabonate (Androsta-1,4-diene-17-carboxylic acid, 17-[(ethoxycarbonyl)oxy]-11-hydroxy-3-oxo-, chloromethyl ester, (11β,17Alpha)-, or an analogue or derivative thereof.

QQ. Histamine H1/H2/H3 Receptor Antagonists

In one aspect of the present invention, an anastomotic connection device is therapeutically associated with a histamine H1/H2/H3 receptor antagonist, where exemplary compounds having this biological activity include: ranitidine (1,1-Ethenediamine, N-[2-[[[5-[(dimethylamino)methyl]-2-furanyl]methyl]thio]ethyl]-N′-methyl-2-nitro-), niperotid ine (N-[2-[[5-[(dimethylamino)methyl]furfuryl]thio]ethyl]-2-nitro-N′-piperonyl-1,1-ethenediamine), famotidine (Propanimidamide, 3-[[[2-[(aminoiminomethyl)amino]-4-thiazolyl]methyl]thio]-N-(aminosulfonyl)-), roxitadine acetate HCI (Acetamide, 2-(acetyloxy)-N-[3-[3-(1-piperidinylmethyl)phenoxy]propyl]-, monohydrochloride ), lafutidine (Acetamide, 2-[(2-furanylmethyl)sulfinyl]-N-[4-[[4-(1-piperidinylmethyl)-2-pyridinyl]oxy]-2-butenyl]-, (Z)-), nizatadine (1,1-Ethenediamine, N-[2-[[[2-[(dimethylamino)methyl]-4-thiazolyl]methyl]thio]ethyl]-N′-methyl-2-nitro-), ebrotidine (Benzenesulfonamide, N-[[[2-[[[2-[(aminoiminomethyl)amino]-4-thiazoly]methyl]thio]ethyl]amino]methylene]-4-bromo-), rupatadine (5H-Benzo[5,6]cyclohepta[1,2-b]pyridine, 8-chloro-6,11-dihyd ro-11-[1-[(5-methyl-3-pyridinyl)methyl]-4-piperidinylidene]-, trihydrochloride-), fexofenadine HCl (Benzeneacetic acid, 4-[1-hydroxy-4-[4(hydroxydiphenylmethyl)-1-piperidinyl]butyl]-Alpha,Alpha-dimethyl-, hydrochloride, or an analogue or derivative thereof.

RR. Macrolide Antibiotics

In one aspect of the present invention, an anastomotic connection device is therapeutically associated with a macrolide antibiotic, where exemplary compounds of this class include: dirithromycin, (Erythromycin, 9-deoxo-11-deoxy-9,11-[imino[2-(2-methoxyethoxy)ethylidene]oxy]-, [9S(R)]-), flurithromycin ethylsuccinate (Erythromycin, 8-fluoro-mono(ethyl butanedioate) (ester)-), erythromycin stinoprate (Erythromycin, 2′-propanoate, compd. with N-acetyl-L-cysteine (1:1) ), clarithromycin (Erythromycin, 6-O-methyl-), azithromycin (9-deoxo-9a-aza-9a-methyl-9a-homoerythromycin-A), telithromycin (3-De((2,6-dideoxy-3-C-methyl-3-O-methyl-Alpha-L-ribo-hexopyranosyl)oxy)- 11,12-dideoxy-6-O-methyl-3-oxo-12,11-(oxycarbonyl((4-(4-(3-pyridinyl)-1H-imidazol-1-yl)butyl)imino))-), roxithromycin (Erythromycin, 9-[O-[(2-methoxyethoxy)methyl]oxime] ), rokitamycin (Leucomycin V, 4B-butanoate 3B-propanoate ), RV-11 (erythromycin monopropionate mercaptosuccinate), midecamycin acetate (Leucomycin V, 3B,9-diacetate 3,4B-dipropanoate ), midecamycin (Leucomycin V, 3,4B-dipropanoate), josamycin (Leucomycin V, 3-acetate 4B-(3-methylbutanoate), or an analogue or derivative thereof.

SS. GPIIb IIIa Receptor Antagonists

In one aspect of the present invention, an anastomotic connection device is therapeutically associated with an GPIIb IIIa receptor antagonist, where exemplary compounds having this biological activity include: tirofiban hydrochloride (L-Tyrosine, N-(butylsulfonyl)-O-[4-(4-piperidinyl)butyl]-, monohydrochloride-), eptifibatide (L-Cysteinamide, N6-(aminoiminomethyl)-N2-(3-mercapto-1-oxopropyl)-L-lysylglycyl-L-Alpha-aspartyl-L-tryptophyl-L-prolyl-, cyclic(1→6)-disulfide, or an analogue or derivative thereof.

TT. Endothelin Receptor Antagonists

In one aspect of the present invention, an anastomotic connection device is therapeutically associated with an endothelin receptor antagonist, where exemplary compounds having this biological activity include: bosentan (Benzenesulfonamide, 4-(1,1-dimethylethyl)-N-[6-(2-hyd roxyethoxy)-5-(2-methoxyphenoxy)[2,2′-bipyrimidin]-4-yl], or an analogue or derivative thereof.

UU. Peroxisome Proliferator-Activated Receptor Agonists

In one aspect of the present invention, an anastomotic connection device is therapeutically associated with a peroxisome proliferators-activated receptor agonist, where exemplary compounds having this biological activity include: gemfibrozil (Pentanoic acid, 5-(2,5-dimethylphenoxy)-2,2-dimethyl-), fenofibrate (Propanoic acid, 2-[4-(4-chlorobenzoyl)phenoxy]-2-methyl-, 1-methylethyl ester ), ciprofibrate (Propanoic acid, 2-[4-(2,2-dichlorocyclopropyl)phenoxy]-2-methyl-), rosiglitazone maleate (2,4-Thiazolidinedione, 5-((4-(2-(methyl-2-pyridinylamino)ethoxy)phenyl)methyl)-, (Z)-2-butenedioate (1:1) ), pioglitazone hydrochloride (2,4-Thiazolidinedione, 5-[[4-[2-(5-ethyl-2-pyridinyl)ethoxy]phenyl]methyl]-, monohydrochloride (+/−)-), etofylline clofibrate (Propanoic acid, 2-(4-chlorophenoxy)-2-methyl-, 2-(1,2,3,6-tetrahydro-1,3-dimethyl-2,6-dioxo-7H-purin-7-yl)ethyl ester ), etofibrate (3-Pyridinecarboxylic acid, 2-[2-(4-chlorophenoxy)-2-methyl-1-oxopropoxy]ethyl ester), clinofibrate (Butanoic acid, 2,2′-[cyclohexylidenebis(4,1-phenyleneoxy)]bis[2-methyl-]), bezafibrate (Propanoic acid, 2-[4-[2-[(4-chlorobenzoyl)amino]ethyl]phenoxy]-2-methyl-), binifibrate (3-Pyridinecarboxylic acid, 2-[2-(4-chlorophenoxy)-2-methyl-1-oxopropoxy]-1,3-propanediyl ester, or an analogue or derivative thereof.

VV. Estrogen Receptor Agents

In one aspect of the present invention, an anastomotic connection device is therapeutically associated with an estrogen receptor agent, where exemplary compounds having this biological activity include: estradiol, 17-β-estradiol, or an analogue or derivative thereof.

WW. Somatostatin Analogues

In one aspect of the present invention, an anastomotic connection device is therapeutically associated with a somatostatin analogue, where exemplary compounds of this class include: angiopeptin, or an analogue or derivative thereof.

XX. Sirolimus and Sirolimus analogues

In one aspect of the present invention, an anastomotic connection device is therapeutically associated with an immunosuppressant such as sirolimus, or a derivative or an analogue thereof. Briefly, sirolimus (also referred to as “rapamycin”) is a macrolide antibiotic. Therapeutically the drug is classified as an immunosuppressant. Its mechanistic classification is as a cell cycle inhibitor and an mTORR (mammalian target of rapamycin) inhibitor. The structure of sirolimus, everolimus, and tacrolimus is provided below:

| Name | Code Name | Company | Structure |
|---|---|---|---|
| Everolimus | SAR-943 | Novartis | See below |
| Sirolimus | AY-22989 | Wyeth | See below |
| Rapamune | NSC-226080 | | |
| Rapamycin | | | |
| Tacrolimus | FK506 | Fujisawa | See below |

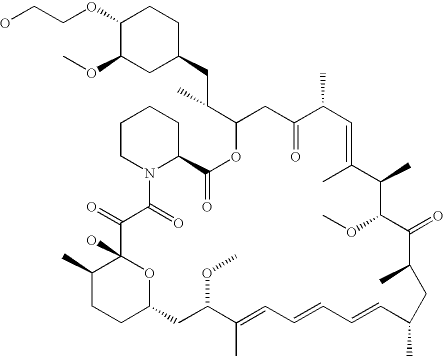

Everolimus 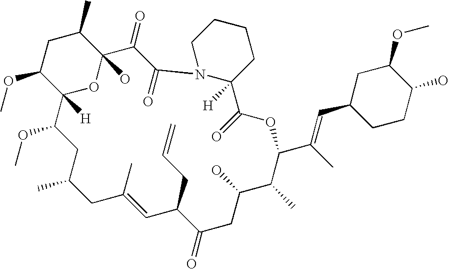
Tacrolimus 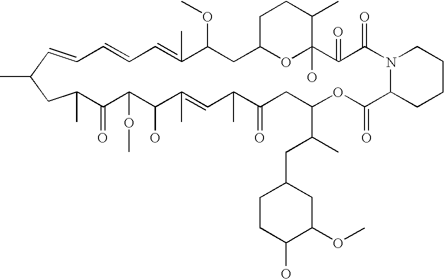
Sirolimus

Further sirolimus analogues and derivatives include tacrolimus and derivatives thereof (e.g., EP0184162B1 and U.S. Pat. No.6,258,823) everolimus and derivatives thereof (e.g., U.S. Pat. No.5,665,772). Further representative examples of sirolimus analogues and derivatives can be found in PCT Publication Nos. WO9710502, WO9641807, WO9635423, WO9603430, WO9600282, WO9516691, WO9515328, WO9507468, WO9504738, WO9504060, WO9425022, WO9421644, WO9418207, WO9410843, WO9409010, WO9404540, WO9402485, WO9402137, WO9402136, WO9325533, WO9318043, WO9313663, WO9311130, WO9310122, WO9304680, WO9214737, and WO9205179. Representative U.S. patents include U.S. Pat. Nos. 6,342,507, 5,985,890, 5,604,234, 5,597,715, 5,583,139, 5,563,172, 5,561,228, 5,561,137, 5,541,193, 5,541,189, 5,534,632, 5,527,907, 5,484,799, 5,457,194, 5,457,182, 5,362,735, 5,324,644, 5,318,895, 5,310,903, 5,310,901, 5,258,389, 5,252,732, 5,247,076, 5,225,403, 5,221,625, 5,210,030, 5,208,241, 5,200,411, 5,198,421, 5,147,877, 5,140,018, 5,116,756, 5,109,112, 5,093,338, and 5,091,389.

YY. Podophyllotoxins

In one aspect of the present invention, an anastomotic connection device is therapeutically associated with a Podophyllotoxin, or a derivative or an analogue thereof. Exemplary compounds of this type are Etoposide or Teniposide, which have the following structures:

| | | |
|---|---|---|
| Eto- poside Teni- poside | R CH₃ 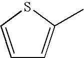 | 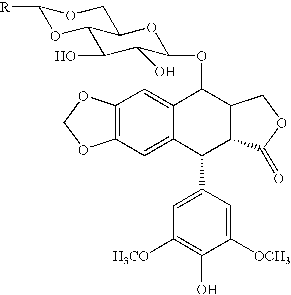 |

ZZ. Angiogenesis Inhibitors

In one aspect of the present invention, an anastomotic connection device is therapeutically associated with an angiogenesis inhibitor, where exemplary compounds having this biological activity include: 2-ME (NSC-659853), PI-88 (D-Mannose, O-6-O-phosphono-Alpha-D-mannopyranosyl-(1-3)-O-Alpha-D-mannopyranosyl-(1-3)-O-Alpha-D-mannopyranosyl-(1-3)-O-Alpha-D-mannopyranosyl-(1-2)- hydrogen sulphate), thalidomide (1H-Isoindole-1,3(2H)-dione, 2-(2,6-dioxo-3-piperidinyl)-), CDC-394, CC-5079, ENMD-0995 (S-3-amino-phthalidoglutarimide), AVE-8062A, Vatalanib, SH-268, Halofuginone hydrobromide), or an analogue or derivative thereof.

AAA. Pyrolidine Antibiotics

In one aspect of the present invention, an anastomotic connection device is therapeutically associated with a pyrolidine antibiotic. A representative example of a pyrolidine antibiotic is anisomycin.

### II. COMPOSITIONS AND FORMULATIONS

Therapeutic agents that are associated with an anastomotic connector device according to the present invention may be formulated with other components in order to provide desired effect. For example, the therapeutic agent may be formulated with a carrier, where the carrier functions to adhere the agent to the anastomotic connector, and/or to affect the rate at which the agent is released from the anastomotic connector. In this regard, a wide variety of carriers may be selected and have either a polymeric or a non-polymeric origin. The polymers and non-polymer based carriers and formulations which are discussed in more detail below, are provided merely by way of example and not by way of limitation.

Within one embodiment of the invention a wide variety of polymers can be utilized to contain and/or deliver one or more of the agents discussed above, including for example both biodegradable and non-biodegradable compositions. Representative examples of biodegradable compositions include albumin, collagen, gelatin, chitosan, hyaluronic acid, starch, cellulose and derivatives thereof (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), alginates, casein, dextrans, polysaccharides, fibrinogen, poly(L-lactide), poly(D,L lactide), poly(L-lactide-co-glycolide), poly(D,L-lactide-co-glycolide), poly(glycolide), poly(trimethylene carbonate), poly(hydroxyva lerate), poly(hydroxybutyrate), poly(caprolactone), poly(alkylcarbonate) and poly(orthoesters), polyesters, poly(hydroxyvaleric acid), polydioxanone, poly(malic acid), poly(tartronic acid), polyanhydrides, polyphosphazenes, poly(amino acids) (e.g., poly(glutamic acid), copolymers of such polymers and blends of such polymers (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986).

In another embodiment, the carrier can be a polyester. Polyesters that can be used include the poly(hydroxyesters). In another embodiment, the polyester can comprise the residues of one or more of the monomers selected from lactide, lactic acid , glycolide, glycolic acid, ε-caprolactone, gamma-caprolactone, hydroxyvaleric acid, hydroxybutyric acid, beta-butyrolactone, gamma-butyrolactone, gamma-valerolactone, Γ-decanolactone, δ-decanolactone, trimethylene carbonate, 1,4-dioxane-2-one or 1,5-dioxepan-2one. These polyesters can be linear or branched materials. The branched materials can be prepared using an initiator that has three or more functional groups that are capable of initiating the ring-opening polymerization process. An example of this type of initiator includes triethanolamine and pentaerythritol.

In another embodiment, the carrier can be poly(alkylene oxide)poly(ester) block copolymers (e.g. X-Y, X-Y-X or Y-X-Y, where X is a polyalkylene oxide [e.g. poly(ethylene glycol), poly(propylene glycol), poly(ethylene oxide), poly(propylene oxide), diblock and triblock copolymers of ethylene oxide and propylene oxide (e.g. Pluronic and Pluronic R polymers by BASF) and Y is a polyester where the polyester can comprise the residues of one or more of the monomers selected from lactide, lactic acid , glycolide, glycolic acid, Γ-caprolactone, gamma-caprolactone, hydroxyvaleric acid, hydroxybutyric acid, beta-butyrolactone, gamma-butyrolactone, gamma-valerolactone, Γ-decanolactone, δ-decanolactone, trimethylene carbonate, 1,4-dioxane-2-one or 1,5-dioxepan-2one [e.g. PLGA, PLA, PCL, polydioxanone and copolymers thereof).

Representative examples of nondegradable polymers include poly(ethylene-co-vinyl acetate) (“EVA”) copolymers, silicone rubber, acrylic polymers (e.g., polyacrylic acid, polymethylacrylic acid, poly(hydroxyethylmethacrylate), polymethylmethacrylate, polyalkylcyanoacrylate), polyethylene, polypropylene, polyamides (e.g., nylon 6,6), poly(styrene-block-isobutylene-block-styrene), polyurethane (e.g., poly(ester urethanes), poly(ether urethanes), poly(ester-urea), poly(carbonate urethanes)), polyethers (e.g., poly(ethylene oxide), poly(propylene oxide), Pluronics and poly(tetramethylene glycol)) and vinyl polymers [e.g., polyvinylpyrrolidone, poly(vinyl alcohol), poly(vinyl acetate phthalate), poly(styrene)] as well as copolymers and blends thereof. Polymers may also be developed which are either anionic (e.g., alginate, carrageenin, carboxymethyl cellulose and poly(acrylic acid), or cationic (e.g., chitosan, poly-L-lysine, polyethylenimine, and poly (allyl amine)) (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995). Exemplary polymeric carriers include poly(ethylene-co-vinyl acetate), polyurethane, hydrophobic cellulose derivatives, poly(caprolactone), poly(valerolactone), polyanhydrides, copolymers of poly(caprolactone) or poly(lactic acid) with a polyethylene glycol (e.g., MePEG), and blends thereof.

Other representative polymers include carboxylic polymers, polyacetates, polyacrylamides, polycarbonates, polyethers, polyesters, polyethylenes, polyvinylbutyrals, polysilanes, polyureas, polyurethanes, poly(ester-amides), poly(ester-imides), poly(ester-ureas), poly(ester-urethane-ureas), poly(anhydride-esters), poly(anhydride-imides) polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxy, melamine, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, polyurethane, polyacrylate, natural and synthetic elastomers, rubber, acetal, nylon, polyester, styrene polybutadiene, acrylic resin, polyvinylidene chloride, polycarbonate, homopolymers and copolymers of vinyl compounds, polyvinylchloride, polyvinylchloride acetate.

Representative examples of patents relating to polymers and their preparation include PCT Publication Nos. WO97/2827, 98/12243, 98/19713, 98/41154, 99/07417, 00/33764, 00/21842, 00/09190, 00/09088, 00/09087, 2001/17575 and 2001/15526 (as well as their corresponding U.S. applications), and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,099,563, 6,106,473, 6,110,483, 6,121,027, 6,156,345, 6,179,817, 6,197,051, 6,214,901, 6,335,029, 6,344,035.

Polymers can be fashioned in a variety of forms, with desired release characteristics and/or with specific desired properties. For example, polymers can be fashioned to release a therapeutic agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp.175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid)-based polymers and derivatives (including, for example, homopolymers such as poly(aminocarboxylic acid), poly(acrylic acid), poly(methyl acrylic acid), copolymers of such homopolymers, and copolymers of poly(acrylic acid) and acrylmonomers such as those discussed above). Other pH sensitive polymers include polysaccharides such as carboxymethyl cellulose, hydroxypropyl-methylcellulose phthalate, hydroxypropyl-methylcellulose acetate succinate, cellulose acetate trimellilate, chitosan and alginates. Yet other pH sensitive polymers include any mixture of a pH sensitive polymer and a water soluble polymer or a water-insoluble polymer.

Likewise, polymers can be fashioned to be temperature sensitive (see, e.g., Chen et al., “Novel Hydrogels of a Temperature-Sensitive Pluronic Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al.L (eds.), Polymers in Medicine III, Elsevier Science Publishers B.V., Amsterdam, 1988, pp.161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers include homopolymers such as poly(N-methyl-N-n-propylacrylamide), poly(N-n-propylacrylamide), poly(N-methyl-N-isopropylacrylamide), poly(N-n-propylmethacrylamide), poly(N-isopropylacrylamide), poly(N, n-diethylacrylamide), poly(N-isopropylmethacrylamide), poly(N-cyclopropylacrylamide), poly(N-ethylmethyacrylamide), poly(N-methyl-N-ethylacrylamide), poly(N-cyclopropylmethacrylamide) and poly(N-ethylacrylamide). Moreover thermogelling polymers may be made by preparing copolymers between (among) monomers of the above, or by combining such homopolymers with other water soluble polymers such as acrylmonomers (e.g., acrylic acid and derivatives thereof such as methylacrylic acid, acrylate and derivatives thereof such as butyl methacrylate, acrylamide, and N-n-butyl acrylamide).

Other representative examples of thermogelling cellulose ether derivatives can be used, such as hydroxypropyl cellulose, methyl cellulose, hydroxypropylmethyl cellulose, ethylhydroxyethyl cellulose, and Pluronics, such as F-127.

A wide variety of forms may be fashioned with the polymers of the present invention, including, for example, rod-shaped devices, pellets, slabs, particulates, micelles, films, molds, sutures, threads, gels, creams, ointments, sprays or capsules (see, e.g., Goodell et al., Am. J. Hosp. Pharm. 43:1454-1461, 1986; Langer et al., “Controlled release of macromolecules from polymers”, in Biomedical Polymers, Polymeric Materials and Pharmaceuticals for Biomedical Use, Goldberg, E. P., Nakagim, A. (eds.) Academic Press, pp.113-137, 1980; Rhine et al., J. Pharm. Sci. 69:265-270, 1980; Brown et al., J. Pharm. Sci. 72:1181-1185, 1983; and Bawa et al., J. Controlled Release 1:259-267, 1985). Agents may be incorporated by dissolution in the polymer, occlusion in the matrices of the polymer, bound by covalent linkages, or encapsulated in microcapsules. Within certain preferred embodiments of the invention, therapeutic compositions are provided in non-capsular formulations, such as coatings microspheres (ranging from nanometers to micrometers in size), pastes, threads or sutures of various size, films, and sprays.

In one embodiment, the carrier can be non-polymeric. These non-polymeric agents can include sucrose derivatives (e.g. sucrose acetate isobutyrate, sucrose oleate), sterols such as cholesterol, stigmasterol, beta-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydoxyapatite, zeolites; and combinations and mixtures thereof.

Representative examples of patents relating to non-polymeric delivery systems and their preparation include U.S. Pat. Nos. 5,736,152; 5,888,533; 6,120,789; 5,968,542; and 5,747,058. These delivery systems may be used to formulate an agent that is associated with an anastomotic connector according to the present invention.

Other compounds which can be utilized to carry and/or deliver the agents provided herein include vitamin-based compositions (e.g., based on vitamins A, D, E and/or K, see, e.g., PCT publication Nos. WO 98/30205 and WO 00/71163) and liposomes (see, U.S. Pat. Nos. 5,534,499, 5,683,715, 5,776,485, 5,882,679, 6,143,321, 6,146,659, 6,200,598, and PCT Publication Nos. WO 98/34597, WO 99/65466, WO 00/01366, WO 00/53231, WO 99/35162, WO 00/117508, WO 00/125223, WO 00/149,268, WO 00/1565438, and WO 00/158455).

Preferably, therapeutic compositions of the present invention are fashioned in a manner appropriate to the intended use. Within certain aspects of the present invention, the therapeutic composition should be biocompatible, and release one or more agents over a period of several days to months. Further, therapeutic compositions of the present invention should preferably be stable for several months and capable of being produced, and maintained under sterile conditions.

Within certain aspects of the present invention, therapeutic compositions may be fashioned in any size ranging from 30 nm to 500 μm, depending upon the particular use. Alternatively, such compositions may also be readily applied as a “spray” which solidifies into a film or coating. Such sprays may be prepared from microspheres or microparticles of a wide array of sizes, including for example, from 0.1 μm to 9 μm, from 10 μm to 30 μm and from 30 μm to 100 μm.

Therapeutic compositions of the present invention may also be prepared in a variety of “paste” or gel forms. For example, within one embodiment of the invention, therapeutic compositions are provided which are liquid at one temperature (e.g., temperature greater than 37° C.) and solid or semi-solid at another temperature (e.g., ambient body temperature, or any temperature lower than 37° C.). Also included are polymers, such as Pluronic F-127, which are liquid at a low temperature (e.g., 4° C.) and a gel at body temperature.

Within yet other aspects of the invention, the therapeutic compositions of the present invention may be formed as a film. Preferably, such films are generally less than 5, 4, 3, 2 or 1 mm thick, more preferably less than 0.75 mm or 0.5 mm thick, and most preferably less than 500 μm. Such films are preferably flexible with a good tensile strength (e.g., greater than 50, preferably greater than 100, and more preferably greater than 150 or 200 N/cm²), good adhesive properties (i.e., readily adheres to moist or wet surfaces), and have controlled permeability.

Within certain embodiments of the invention, the therapeutic compositions can also comprise additional ingredients such as surfactants (e.g., Pluronics such as F-127, L-122, L-92, L-81, and L-61), plasticizers (for example, triacetin, trietyl citrate, glycerin, diethyl phthalate, poly(ethylene glycol), agents to reduce tackiness and leveling agents.

Within certain embodiments of the invention, the therapeutic agent or carrier can also comprise radio-opaque, echogenic materials and magnetic resonance imaging (MRI) responsive materials (i.e., MRI contrast agents) to aid in visualization of the device under ultrasound, fluoroscopy and/or MRI. For example, a device may be made with or coated with a composition which is echogenic or radiopaque (e.g., made with echogenic or radiopaque with materials such as powdered tantalum, tungsten, barium carbonate, bismuth oxide, barium sulfate, Metrazimide, lopamidol, lohexol, lopromide, lobitridol, lomeprol, lopentol, loversol, loxilan, Iodixanol, lotrolan, Acetrizoic Acid derivatives, Diatrizoic Acid derivatives, lothalamic Acid derivatives, loxithalamic Acid derivatives, Metrizoic Acid derivatives, lodamide, lypophylic agents, lodipamide and loglycamic Acid or, by the addition of microspheres or bubbles which present an acoustic interface). For visualization under MRI, contrast agents (e.g., Gadolinium (III) chelates or iron oxide compounds) may be incorporated into or onto the device, such as, for example, as a component in a coating or within the void volume of the device (e.g., within a lumen, reservoir, or within the structural material used to form the device).

Within further aspects of the present invention, polymers are provided which are adapted to contain and release a hydrophobic compound, the carrier containing the hydrophobic compound in combination with a carbohydrate, protein or polypeptide. Within certain embodiments, the polymeric carrier contains or comprises regions, pockets or granules of one or more hydrophobic compounds. For example, within one embodiment of the invention, hydrophobic compounds may be incorporated within a matrix which contains the hydrophobic compound, followed by incorporation of the matrix within the polymeric carrier. A variety of matrices can be utilized in this regard, including for example, carbohydrates and polysaccharides, such as starch, cellulose, dextran, methylcellulose, and hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver the agents described herein include: hydroxypropyl β-cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), devices (Jampel et al., Invest. Ophthalm. Vis. Science 34(11): 3076-3083, 1993; Walter et al., Cancer Res. 54:22017-2212, 1994), nanoparticles (Violante and Lanzafame PAACR), nanoparticles—modified (U.S. Pat. No.5,145,684), nanoparticles (surface modified) (U.S. Pat. No. 5,399,363), taxol emulsion/solution (U.S. Pat. No. 5,407,683), micelle (surfactant) (U.S. Pat. No. 5,403,858), synthetic phospholipid compounds (U.S. Pat. No. 4,534,899), gas borne dispersion (U.S. Pat. No. 5,301,664), foam, spray, gel, lotion, cream, ointment, dispersed vesicles, particles or droplets, solid- or liquid-aerosols, microemulsions (U.S. Pat. No. 5,330,756), polymeric shell (nano- and micro-capsule) (U.S. Pat. No. 5,439,686), taxoid-based compositions in a surface-active agent (U.S. Pat. No. 5,438,072), liquid emulsions (Tarr et al., Pharm Res. 4:62-165, 1987), nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994) and devices (U.S. Pat. No. 4,882,168).

Within certain embodiments of the invention, the therapeutic agent may be chemically modified to form a prodrug. This prodrug can then be incorporated directly into or onto the device or this prodrug may further comprise a carrier, as described above, and this combination can be incorporated into or onto the device. For example a therapeutic agent comprising a hydroxyl group, may be covalently bound to a carrier that comprised a carboxylic acid functional group. Paclitaxel, for example, may be covalently bound to a poly(glutamic acid) or an acrylic acid polymer of copolymer.

Within certain embodiments of the invention, a carrier that comprises functional groups can be applied or incorporated into or onto the device. The therapeutic agent that has the ability to covalently bind to these functional groups on the carrier can then be covalently bound to the carrier. A linker or spacer group can also be used to attached the therapeutic agent to the carrier. In the preferred embodiment, the therapeutic agent is covalently bound through a bond or linker that can undergo hydrolysis, enzymatic degradation or a combination thereof.

The agents provided herein can also be formulated as a sterile composition (e.g., by treating the composition with ethylene oxide or by irradiation (e.g. ionizing radiation such as gamma radiation or electron-beam radiation), packaged with preservatives or other suitable excipients suitable for administration to humans. Similarly, the devices provided herein (e.g., coated catheter) may be sterilized and prepared suitable for deviceation (e.g., insertion, implantation, and the like) into humans.

In various aspects of the invention, the agent is formulated into a therapeutic coating, where the coating is placed on the anastomotic connector. In a preferred embodiment, the therapeutic coating has one or more of the following characteristics: (a) the ability to reduce, inhibit or prevent SMC proliferation; (b) the ability to reduce, inhibit or prevent SMC migration (c) the ability to reduce, inhibit or prevent the production of extracellular matrix (d) the ability to reduce, inhibit or prevent the inflammatory response of white blood cells to an implanted foreign body and (e) the ability to reduce, inhibit or prevent the development of thrombus at the anastomotic site.

### III. ANASTOMOTIC CONNECTOR DEVICES

As noted above, the present invention provides anastomotic connector devices which release a desired therapeutic agent. Within preferred embodiments such devices are capable of reducing the incidence of stenosis/restenosis at the proximal and/or distal anastomosis. Since it is difficult to predict in advance which anastomoses will develop clinically significant stenosis/restenosis, any anastomotic connector device can benefit from a therapeutic coating capable of reducing the incidence of neointimal hyperplasia. Provided below are (A) general methods for making anastomotic devices which release one or more desired therapeutic agents, and (B) illustrative embodiments of anastomotic connector devices that release a desired therapeutic agent.

A. General Methods For Making Anastomotic Devices Which Release One Or More desired Therapeutic Agents

The anti-scarring agent or composition that comprises the anti-scarring agent may be associated with the device in a variety of manners. For example, the agent, or composition comprising the agent, may be impregnated into, affixed (e.g., grafted) to, coupled to, connected to, disposed on or within, attached to, adhered to, bonded to, adjacent to, entrapped in, absorbed in, or adsorbed on any portion of the device or the entire device. In a preferred aspect, the agent is releasable from the device, and is released from the device after the device has been inserted into the host.

Thus, in one embodiment of the invention a desired therapeutic agent such as an anthracycline (e.g., doxorubicin, mitoxantrone and analogues or derivatives thereof), a taxane (e.g., paclitaxel and analogues or derivatives thereof), sirolimus (also known as Rapamycin or Rapamune), as well as analogues and derivatives of Sirolimus such as, but not restricted to, everolimus and tacrolimus (also known as FK506), and/or a podophyllotoxin are formulated into a coating applied to the surface of the anastomotic connector device. The drug(s) can be applied to all or a portion of the anastomotic connector device in several manners: (a) as a polymeric and/or non-polymeric coating applied to the surface of the intravascular portion of the anastomotic connector device; (b) as a polymeric and/or non-polymeric coating applied to the extravascular (adventitial or abluminal) and/or intravascular surface of the anastomotic connector device; (c) incorporated into the constituent materials which comprise the anastomotic connector devices (e.g., metals, polymers, ceramics); (d) applied to the adventitial surface of the anastomosis (e.g., as an injectable, paste, gel or mesh applied during the procedure); (e) applied to the endoluminal surface of the anastomosis (e.g., as an injectable, paste, gel or mesh applied during the procedure—also known as “endoluminal paving”); (f) injected into the lumen of the vessels (locally or systemically) in solution as an infusate; (g) incorporated into, or applied as a coating, to a synthetic vascular graft; (h) injected into the pericardial sac in solution as an infusate or as a sustained release preparation (i) injected into the walls of vessels (graft or artery) in solution and/or as a sustained release preparation; (j) directly onto the surface of the device or absorbed into the device, and (j) any combination of the aforementioned.

In one aspect, an anastomotic connector may include a plurality of reservoirs within its structure, each reservoir configured to house and protect a therapeutic drug. The reservoirs may be formed from divets in the device surface or micropores or channels in the device body. In one aspect, the reservoirs are formed from voids in the structure of the device. The reservoirs may house a single type of drug or more than one type of drug. The drug(s) may be formulated with a carrier (e.g., a polymeric or non-polymeric material) that is loaded into the reservoirs. The filled reservoir can function as a drug delivery depot which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount of drug that is released from the substrate. The multi-layered carrier may further include a barrier layer that prevents release of the drug(s). The barrier layer can be used, for example, to control the direction that the drug elutes from the void.

In certain embodiments, the device may be sealed to the target vessel to prevent fluid leaks using a surgical sealant, such as COSEAL (crosslinked material produced by the reaction of pentaerythritol poly(ethylene glycol)ether tetra-sulfhydryl] (4-armed thiol PEG) and pentaerythritol poly(ethylene glycol)ether tetra-succinimidyl glutarate] (4-armed NHS PEG ) (from Cohesion Technologies, Palo Alto, Calif.) or a tissue adhesive, such as a cyanoacrylate (octyl cyanoacrylate, n-butyl cyanoacrylate, methoxypropyl cyanoacrylate, ethyl cyanoacrylate) or crosslinked methylated collagen-poly(ethylene glycol) material (see, e.g., U.S. Pat. Nos., 5,874,500; 5,936,035; 6,273,114; 6,312,725; 6,495,127 and PCT Publication Nos. WO 2004/028547.

Particularly preferred agents which are utilized within the context of the present invention should be used at concentrations less than that 10%, 5%, or even 1% of the concentration typically used in chemotherapeutic (i.e., systemic) applications (see Goodman and Gilman's The Pharmacological Basis of Therapeutics. Editors J. G. Hardman, L. L. Limbird. Consulting editor A. Goodman Gilman Tenth Edition. McGraw-Hill Medical publishing division. 10th edition, 2001).

In addition to the above-noted therapeutic agents, one or more of the desired therapeutic agents can be combined with, or alternatively, coated or otherwise released separately from all or a portion of the anastomotic device. In another embodiment, the therapeutic agent and/or the carrier may further comprise agents that are anti-inflammatory, antiplatelet, anti-thrombotic, antimicrobial and/or antibacterial. Representative examples of anti-thrombotic and/or antiplatelet agents include heparin, heparin fragments, heparin complexes (e.g., benzalkonium heparinate, tridodecylammonium heparinate), dextran sulphate, danaparoid, lepirudin, hirudin, AMP, adenosine, 2-chloroadenosine, aspirin, phenylbutazone, indomethacin, meclofenamate, hydrochloroquine, dipyridamole, iloprost, ticlopidine, clopidogrel, abcixamab, eptifibatide, tirofiban, streptokinase, and/or tissue plasminogen activator), to further enhance efficacy.

Finally, it should be noted that the devices should preferably be provided in sterile form, and suitable for use in humans.

Drug-coating of, or drug incorporation into, the anastomotic connector device allows sufficient levels of the desired drug(s) or agent(s) to be achieved locally, thus reducing the incidence of stenosis/restenosis at the anastomotic site, while producing negligible systemic exposure to the drugs. Although for some agents polymeric carriers are not required for attachment of the drug to the anastomotic connector device, several polymeric carriers are particularly suitable for use in this embodiment. Exemplary are polymeric carriers such as polyurethanes (e.g., CHRONOFLEX AL and CHRONOFLEX AR (CT Biomaterials), HYDROMED640 (CT Biomaterials), HYDROSLIP C (CT Biomaterials), HYDROTHANE AL (CT Biomaterials), Bionate 80A (PTG Medical LLC)), acrylic or methacrylic copolymers (e.g., poly(ethylene-co-acrylic acid), cellulose-derived polymers (e.g., nitrocellulose RS, SS nitrocellulose, cellulose acetate butyrate, cellulose acetate propionate), acrylate and methacrylate copolymers (e.g., poly(hydroxymetharylate), poly(ethylene-co-vinyl acetate) as well as blends thereof.

In one embodiment, all or a portion of the anastomotic connector device, e.g., that portion of the device that is in contact with the tissue at the anastomotic site or that resides within the lumen of the device, is coated with a primer (bonding) layer and a drug release layer, as described in U.S. patent application entitled, “Stent with Medicated Multi-Layer Hybrid Polymer Coating,” filed Sep. 16, 2003 (U.S. Ser. No.10/662,877).

In order to develop a hybrid polymer delivery system for targeted therapy, it is desirable to be able to control and manipulate the properties of the system both in terms of physical and drug release characteristics. The active agents can be imbibed into a surface hybrid polymer layer, or incorporated directly into the hybrid polymer coating solutions. Imbibing drugs into surface polymer layers is an efficient method for evaluating polymer-drug performance in the laboratory, but for commercial production it may be preferred for the polymer and drug to be premixed in the casting mixture. Greater efficacy can be achieved by combining the two elements in the coating mixtures in order to control the ratio of active agent to polymer in the coatings. Such ratios are important parameters to the final properties of the medicated layers, i.e., they allow for better control of active agent concentration and duration of pharmacological activity.

Typical polymers used in the drug-release system can include water-insoluble cellulose esters, various polyurethane polymers including hydrophilic and hydrophobic versions, hydrophilic polymers such as polyethylene glycol (PEG), polyethylene oxide (PEO), polyvinylpyrrolidone (PVP), PVP copolymers such as vinyl acetate, hydroxyethyl methacrylate (HEMA) and copolymers such as methylmethacrylate (PMMA-HEMA), and other hydrophilic and hydrophobic acrylate polymers and copolymers containing functional groups such as carboxyl and/or hydroxyl.

Cellulose esters such as cellulose acetate, cellulose acetate propionate, cellulose acetate butyrate, cellulose acetate phthalate, and cellulose nitrate may be used. In one aspect of the invention, the therapeutic agent is formulated with a cellulose ester. Cellulose nitrate is a preferred cellulose ester because of its compatibility with the active agents and its ability to impart non-tackiness and cohesiveness to the coatings. Cellulose nitrate has been shown to stabilize entrapped drugs in ambient and processing conditions. Various grades of cellulose nitrate are available and may be used in a coating on an anastomotic connector, including cellulose nitrate having a nitrogen content=11.8-12.2%. Various viscosity grades, including 3.5, 0.5 or 0.25 seconds, may be used in order to provide proper rheological properties when combined with the coating solids used in these formulations. Higher or lower viscosity grades could be used. However, the higher viscosity grades can be more difficult to use because of their higher viscosities. Thus, the lower viscosity grades, such as 3.5, 0.5 or 0.25 seconds, are generally preferred. Physical properties such as tensile strength, elongation, flexibility, and softening point are related to viscosity (molecular weight) and can decrease with the lower molecular weight species, especially below the 0.25 second grades.

The cellulose derivatives comprise hydroglucose structures. Cellulose nitrate is a hydrophobic, water-insoluble polymer, and has high water resistance properties. This structure leads to high compatibility with many active agents, accounting for the high degree of stabilization provided to drugs entrapped in cellulose nitrate. The structure of nitrocellulose is given below:

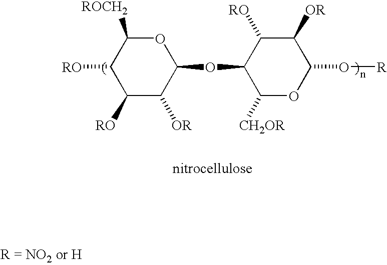

Cellulose nitrate is a hard, relatively inflexible polymer, and has limited adhesion to many polymers that are typically used to make medical devices. Also, control of drug elution dynamics is limited if only one polymer is used in the binding matrix. Accordingly, in one embodiment of the invention, the therapeutic agent is formulated with two or more polymers before being associated with the anastomotic connector. In one aspect, the agent is formulated with both polyurethane and cellulose nitrate to provide a hybrid polymer drug loaded matrix. Polyurethanes provide the hybrid polymer matrix with greater flexibility and adhesion to the anastomotic connector, particularly when the connector has been pre-coated with a primer. Polyurethanes can also be used to slow or hasten the drug elution from coatings. Aliphatic, aromatic, polytetramethylene ether glycol, and polycarbonate are among the types of polyurethanes, which can be used in the coatings. In one aspect, an anti-scarring agent (e.g., paclitaxel) may be incorporated into a carrier that includes a polyurethane and a cellulose derivative. A heparin complex, such as benzalkonium heparinate or tridodecylammonium heparinate), may optionally be included in the formulation.

From the structure below, it is possible to see how more or less hydrophilic polyurethane polymers may be created based on the number of hydrophilic groups contained in the polymer structures. In one aspect of the invention, the anastomotic connector is associated with a formulation that includes therapeutic agent, cellulose ester, and a polyurethane that is water-insoluble, flexible, and compatible with the cellulose ester.

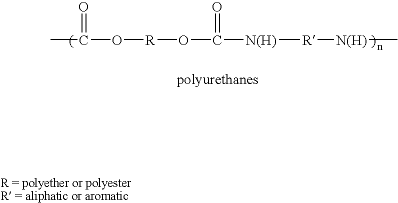

Polyvinylpyrrolidone (PVP) is a polyamide that possesses unusual complexing and colloidal properties and is essentially physiologically inert. PVP and other hydrophilic polymers are typically biocompatible. PVP may be incorporated into drug loaded hybrid polymer compositions in order to increase drug release rates. In one embodiment, the concentration of PVP that is used in drug loaded hybrid polymer compositions can be less than 20%. This concentration would not make the layers bioerodable or lubricious. In general, PVP concentrations from <1% to greater than 80% are deemed workable. In one aspect of the invention, the therapeutic agent that is associated with an anastomotic connector is formulated with a PVP polymer.

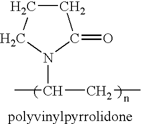

Acrylate polymers and copolymers including polymethylmethacrylate (PMMA) and polymethylmethacrylate hydroxyethyl methacrylate (PMMA/HEMA) are known for their biocompatibility as a result of their widespread use in contact and intraocular lens applications. This class of polymer generally provokes very little smooth muscle and endothelial cell growth, and very low inflammatory response (Bar). These polymers/copolymers are compatible with drugs and the other polymers and layers of the instant invention. Thus, in one aspect, the anastomotic connector device of the present invention is associated with a composition that comprises a therapeutic agent as described above, and an acrylate polymer or copolymer.

Methylmethacrylate hydroxyethylmethacrylate copolymer

The drug-loaded coatings can be prepared as coating solutions in organic solvents. The solutions are non-reactive and can have a shelf life of up to 18 months when stored at room temperature. Simple procedures such as dipping or spraying, followed by air-drying, can be used to apply the drug-containing compositions to the anastomotic connectors. Drying the devices at elevated temperatures (e.g., at about 40° C. to about 120° C.) can remove the residual solvents to produce biocompatible surface layers of approximately 0.3 to 30 microns thick. The drying process can also involve subjecting the coated device to reduced pressure (i.e., vacuum). Once dried, the surface layers are stable for substantially the life of the sterile packaging, generally three to five years, depending on the drug(s) entrapped in polymer layer, and on the storage conditions.

It is recognized in the art that many such drug-releasing compositions may not adhere satisfactorily to some substrates for example, metals and certain plastics such as silicones, polyolefins like polyethylene and polypropylene, certain polyamides, polytetrafluorethylene (TEFLON®), for example. It is necessary in many cases to use various pretreatments or precoats on such surfaces in order to enable the drug-release layer(s) to adhere satisfactorily. Pretreatments such as corona discharge or ionizing plasma are known to those having ordinary skill in the art. Such treatments also include various primer coatings that enable the drug elution layer to bond to the device surface. Furthermore, an intermediate layer may be disposed on the pretreated or primer treated device surface in order to improve the uniformity and/or bonding of the drug eluting layer. The intermediate layer may be comprised of a different polymeric composition than either the primer layer or the drug eluting layer. The drug eluting layer may actually consist of multiple layers disposed serially. Some of the drug eluting layers may contain different polymeric compositions and/or drugs and/or drug concentrations than other drug eluting layers in the composite. Such composite constructs are used to achieve the desired drug elution profile for one or more drug(s).

The polymers used in the primer layer may be cross-linkable and the coating may comprise a cross-linker for the polymers, such as epoxy resin, melamine resin, other amino resin, and phenolic resins. The polymers may be selected from, for example, a carboxyl function acrylic polymer, hydroxyl function acrylic polymer, amine function acrylic polymer, methylol function, and amide function acrylic polymer. They may be a cross-linkable acrylic selected from methylmethacrylate, butylmethacrylate, isobutylmethacrylate, ethylmethacrylate, methylacrylate, ethylacrylate, butyl acrylate acrylic acid, methacrylic acid, styrene methacrylate, and styrene acrylate, and copolymers thereof, and other non-acrylic polymers such as polyurethanes, polycarbonate-urethanes, silicone-urethanes, aliphatic polyurethanes, polyvinyl pyridine copolymers, polyethylene glycol, polyethylene oxide, polyamide copolymer, polyimide copolymer, other polymers known to those of skill in the art may be used in the primer layer.

The primer layer may comprise hydrophobic polymers that are preferably water-insoluble polymers and do not significantly react with the hydrophilic polymers in solution, have low water absorption, provide a high degree of flexibility, and have improved bonding to anastomotic connector substrates. Suitable commercial products that may be used include acrylics such as ACRYLOID (Rohm & Haas) AT-63, AT-51, AT-81, WR-97; ethylene acrylic acid copolymers such as PRIMACOR (DOW) 5989, 5990; melamine resins such as CYMEL hexamethoxymethylmelamine (CYTEC Industries) 303, 370, 380; epoxies such as EPON (Shell) 1001; and polyvinylbutyral such as BUTVAR B-79 (Monsanto), polyurethanes such TECOFLEX 93A, CHRONOFLEX AR. The preferred acrylic stabilizing polymers include reactive groups such as hydroxyl or carboxyl that can react with epoxies but do not render the polymer hydrophilic.

In one embodiment, the coating may include a hydrophilic polymer used in the primer and/or the drug reservoir layer(s), such as a water soluble polyolefin such as a hydrophilic vinyl polymer having polar pendant groups, a polyacrylate or methacrylate having hydrophilic esterifying groups, a polyether, a polyethylene glycol, or other polymer with hydrophilic characteristics as known in the art. In one aspect of the invention, the hydrophilic polymer is PVP or PVP/vinyl acetate such as PVP/VA (GAF) E-335 and E-635.

The hydrophilic polymer component may be of any of the classes discussed in Concise Encyclopedia of Polymer Science and Engineering, Kroschwitz, ed. (Wiley 1990), pp. 458-59, which is incorporated herein by reference. Polymers such as polyvinylpyrrolidone, polyethylene glycol, polyethylene oxide, or polyvinyl alcohol are acceptable, alone or in combination. Examples of suitable hydrophilic polymers include homopolymers or copolymers of the following compounds: polyolefins such as vinyl polymers having polar pendant groups, N-vinylpyrrolidone, N-vinyllactam, N-vinyl butyrolactam, N-vinyl caprolactam, sodium styrene sulfonate monomer, 2-acrylamido-2-methylpropane sulfonic acid, sodium vinyl sulfonate, vinyl pyridine, acrylates or methacrylates having hydrophilic esterifying groups. Other hydrophilic polymers include polyethers, polyethylene glycol, polysaccharides, hydrophilic polyurethanes, polyhydroxyacrylates, polymethacrylates, and copolymers of vinyl compounds and hydroxyacrylates or acrylic acid, so long as the appropriate hydrophilicity is present. Other examples include dextran, xanthan, hydroxypropyl cellulose, methyl cellulose, polyacrylamide, and polypeptides. Other hydrophilic components are known to persons of skill in the art.

The coating may include an acrylic compound, e.g., polymers and copolymers of acrylic acid and methacrylic acid and esters thereof, as defined for example in ACRYLOID Thermoplastic Acrylic Ester Resins for Industrial Finishing, Rohm & Haas, Bulletin 82A37 (1987), including cross-linkable acrylics with at least one component containing carboxyl, hydroxyl, amide, or methylol groups. The following ACRYLOID polymers with functional groups given are preferred: AT-51 (hydroxyl), AT-63 (hydroxyl), AT-81 (carboxyl), and WR-97 (hydroxyl). Cross-linkable acrylic emulsions such as RHOPLEX B-15J (Rohm & Haas), and styrene acrylic emulsions such as AROLON 820-W-49 (Reichhold) may also be used.

A variety of polymers may be used, e.g., epoxy resins, particularly cured epoxy polymers such as EPOTUF 38-505 (Reichhold), and preferably those cured with polyamide, such as EPOTUF 37-618 (Reichhold), vinyl polymers, particularly vinyl acetate, vinyl acetals such as polyvinyl butyral, and ethylene vinyl acetate copolymers. Other appropriate polymers having the requisite characteristics will be apparent to persons of ordinary skill. The polymers preferably, but not necessarily, contain reactive groups or points of reactivity such as hydroxyls, mono-, di- and tertiary amines, acids such as carboxyl, amides, or other groups which represent points of chemical reactivity. In the case of the acrylics, this is referred to as having a “functionality” that is cross-linkable. The polymers and points of chemical reactivity are able to form attractive forces such as hydrogen bonding toward the medical device surface, and also toward the hydrophilic polymer and/or bioactive agent. Such bonds are very strong, and provide desirable adhesion and flexibility to the coating presumably without requiring covalent, ionic, or other links.

Polymers with reactive groups are preferred in the primer layer with anastomotic connectors, which present a metal substrate. However, polymers lacking such groups such as acrylic or styrene copolymers may also be used effectively. The reactive groups can also react to form a cross-linked matrix or help to form a cross-linked matrix. If desired, cross-linkers such as urea resins, melamines, isocyanates, phenolics, and others may be incorporated to interact with the points of chemical reactivity on the polymer chains to cross-link the polymers of the invention with themselves. Alternatively, cross-linkers may react with themselves as stabilizing polymers to form a cross-linked matrix in which the hydrophilic polymer is enmeshed, resulting in an adherent, flexible coating. Cross-linking is useful in promoting effective adhesion by ensuring that the solvents do not attack and degrade the polymer layer excessively when subsequent layers are applied.

The drug reservoir layer may comprise mixtures of polymers having various degrees of hydrophilicity. A relatively more hydrophobic polymer may be selected from cellulose esters such as cellulose nitrate, polycarbonate-urethanes, acrylate polymers and copolymers with or without functional groups such as those previously cited in this disclosure. Relatively more hydrophilic polymers may be selected from vinyl polymers with hydrophilic pendant groups such PVP and its copolymers, polyethylene glycol, polyethylene oxide, HEMA, HEMA-acrylate and methacrylate copolymers, and other hydrophilic polymers/copolymers previously cited in this disclosure.

The total amount of eluted drug, the rate of elution and length of elution time is influenced by the amount of, thickness of and/or the number of coatings of the drug releasing layer, the hydrophilicity of the layer(s), the solubility of the drug in the carrier, the use of surface barrier layers (specific coatings or modification of the surface of the drug/carrier layer), the use of additives, such as plasticizers, and the solubility of the drug(s) in the medium into which it/they are being released. The rate of drug elution can be measured using methods that are well known in the art, including HPLC, UV spectroscopy and measurement (counting) of radioactivity from radiolabeled drugs

The present invention provides formulations that can produce coatings which are extremely durable, even when subjected to adhesion and flexing tests. The coatings are non-reactive with living tissue and, in certain embodiments, are non-thrombogenic in blood, particularly when heparin complexes are included in the formulation. Certain formulations provide coatings that are not substantially biodegradable.

The coating may form a continuous or discontinuous surface layer on the device and may cover all or a portion of the device surface. The coatings may be applied to the surface of an anastomotic device with sufficient thickness and permanence to retain the coating's desirable qualities throughout the useful life of the coated device. The coatings of the invention may be thin, on the order of 0.9 to 100 microns, preferably less than about 50 or more preferably less than about 30 microns.

The coatings preferably adhere to a wide variety of substrates and are resistant to removal on prolonged soaking in aqueous fluids from a variety of polymeric and metallic substrates and other surfaces that are generally considered as presenting adherence problems, including polyethylene, polypropylene, polyamide (nylon), polyester, polyurethane, poly(vinyl chloride), silicone, polycarbonate, and metals and metal alloys, such as stainless steel, platinum, gold, nickel, titanium, nickel-titanium alloys, and chrome.

The coatings may be applied by various techniques such as dip, pour, pump, spray, brush, spin, wipe, solvent casting, contact or screen printing, ink jet, electrodeposition, powder, web, slot die, ion-beam and laser deposition, lamination, self-assembly or other methods known to those skilled in the art (see, e.g., Design and Applications of Hydrophilic Polyurethanes, by T. Thomson, Technomic Publishing Co, Inc. 2000; R. Narayarni, K. P. Rao. J. Biomat. Sci. Polymer Edn. Vol 7, No 1, pp. 39-48; V. A. Lee, R. G. Craig, F. E. Filisko, R. Zand. J. Biomed Materials Res, Vol 31, 51-62; Transactions of Society for Biomaterials, Volume 111, 1998 and Volum 12, 1999; Lubricating Polymer Surfaces, by Y. Ikada, Y. Uyama. Technomic Publishing Co. 1993; Dipcoating, p. 46-47, p.49; Laboratory Handbook of Organic Coatings, by M. W. Urban. Global Press 1997; Coatings Technology Handbook, 2^{nd} edition, edited by D. Satas, A. A. Tracton. Marcel Dekker, Inc. 2001; Course notes, MIT. May 18, 2003. (http://thinkcycle. media. mit.edu/thinkcycle/notes/noveldesignforendotrachealtube html); and “Medical Device Manufacturing by Laser Micromachining Technology”, 1999. (http://www. resonetics.com/MDmfg.htm)).

For certain types of devices, it may be necessary to treat the surface with gas plasma or other ionizing treatment to promote adhesion to the substrate. For example, the device may be modified by coating with a polymer, surface treated by plasma treatment, flame treatment, corona treatment, surface oxidation or reduction, surface etching, mechanical smoothing or roughening, or grafting prior to the coating process.

As described above, a range of polymeric and non-polymeric materials can be used to incorporate the therapeutic agent onto or into a device. Coating of the device with these therapeutic agent containing compositions or with the therapeutic agent only is one process that can be used to incorporate the therapeutic agent into or onto the device and within the various coating processes, there can be several different methods for incorporating the drug into or onto the device.

1. Dip coating

Dip coating is one coating process that can be used to associate the anti-scarring agent with the anastomotic connector device. In one embodiment, the therapeutic agent is dissolved in a solvent for the therapeutic agent and is then coated onto the device. A variety of solvents may be used and are described below.

The solvent may be an inert solvent for the device such that the solvent does not dissolve the medical device to any great extent and is not absorbed by the device to any great extent. The device can be immersed, either partially or completely, in the therapeutic agent/solvent solution for a specific period of time. The rate of immersion into the therapeutic agent/solvent solution can be altered (e.g. 0.001 cm per sec to 50 cm per sec). The device can then be removed from the solution. The rate at which the device can be withdrawn from the solution can be altered (e.g. 0.001 cm per sec to 50 cm per sec). The coated device can be air-dried. The dipping process can be repeated one or more times depending on the specific application. The device can be dried under vacuum to reduce residual solvent levels. This process will result in the therapeutic agent being coated on the surfave of the device.

The solvent may be one that will not dissolve the device but will be absorbed by the device. These solvents can thus swell the device to some extent. The device can be immersed, either partially or completely, in the therapeutic agent/solvent solution for a specific period of time (seconds to days). The rate of immersion into the therapeutic agent/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the therapeutic agent being adsorbed into the medical device. The therapeutic agent may also be present on the surface of the device. The amount of surface associated therapeutic agent may be reduced by dipping the coated device into a solvent for the therapeutic agent or by spraying the coated device with a solvent for the therapeutic agent.

The solvent may be one that will be absorbed by the device and that will dissolve the device. The device can be immersed, either partially or completely, in the therapeutic agent/solvent solution for a specific period of time (seconds to hours). The rate of immersion into the therapeutic agent/solvent solution can be altered (e.g. 0.001 cm per sec to 50 cm per sec). The device can then be removed from the solution. The rate at which the device can be withdrawn from the solution can be altered (e.g. 0.001 cm per sec to 50 cm per sec). The coated device can be air-dried. The dipping process can be repeated one or more times depending on the specific application. The device can be dried under vacuum to reduce residual solvent levels. This process will result in the therapeutic agent being adsorbed into the medical device as well as being surface associated. In a preferred embodiment, the exposure time of the device to the solvent would be such that the device does not undergo significant permantent dimensional changes. The therapeutic agent may also be present on the surface of the device. The amount of surface associated therapeutic agent may be reduced by dipping the coated device into a solvent for the therapeutic agent or by spraying the coated device with a solvent for the therapeutic agent.

In one embodiment, the therapeutic agent and a polymer are dissolved in a solvent, for both the polymer and the fibrosis-inhibiting agent, and are then coated onto the device.

A suspension of the therapeutic agent in a polymer solution can be prepared. The suspension can be prepared by choosing a solvent that can dissolve the polymer but not the therapeutic agent or a solvent that can dissolve the polymer and in which the therapeutic agent is above its solubility limit. In similar processes described above, a device can be dipped into the suspension of the fibrosis-inhibiting agent and polymer solution such that the device is coated with the polymer composition containing the agent.

2. Spray Coating

Spray coating is another coating process that can be used to associate the agent with the anastomotic connector device. In the spray coating process, a solution or suspension of the therapeutic agent, with or without a polymeric or non-polymeric carrier, is nebulized and directed to the device to be coated by a stream of gas. One can use spray devices such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part #14545 and 14654, Alltech Associates, Inc. Deerfield, Ill., and ultrasonic spray devices (for example those available from Sono-Tek, Milton, N.Y.). One can also use powder sprayers and electrostatic sprayers.

In one embodiment, the therapeutic agent is dissolved in a solvent for the fibrosis agent and is then sprayed onto the device. The solvent may be an inert solvent for the device such that the solvent does not dissolve the medical device to any great extent and is not absorbed by the device to any great extent. The device can be held in place or the device can be mounted onto a mandrel or rod that has the ability to move in an X, Y or Z plane or a combination of these planes. Using one of the above described spray devices, the device can be spray coated such that the device is either partially or completely coated with the therapeutic agent/solvent solution. The rate of spraying of the therapeutic agent/solvent solution can be altered (e.g. 0.001 mL per sec to 10 mL per sec) to ensure that a good coating of the therapeutic agent is obtained. The coated device can be air-dried. The spray coating process can be repeated one or more times depending on the specific application. The device can be dried under vacuum to reduce residual solvent levels. This process will result in the therapeutic agent being coated on the surface of the device.

The solvent may be one that will not dissolve the device but will be absorbed by the device. These solvents can thus swell the device to some extent. The device can be spray coated, either partially or completely, in the therapeutic agent/solvent solution. The rate of spraying of the therapeutic agent/solvent solution can be altered (e.g. 0.001 mL per sec to 10 mL per sec) to ensure that a good coating of the therapeutic agent is obtained. The coated device can be air-dried. The spray coating process can be repeated one or more times depending on the specific application. The device can be dried under vacuum to reduce residual solvent levels. This process will result in the therapeutic agent being adsorbed into the medical device. The therapeutic agent may also be present on the surface of the device. The amount of surface associated therapeutic agent may be reduced by dipping the coated device into a solvent for the therapeutic agent or by spraying the coated device with a solvent for the therapeutic agent.

The solvent may be one that will be absorbed by the device and that will dissolve the device. The device can be spray coated, either partially or completely, in the therapeutic agent/solvent solution. The rate of spraying of the therapeutic agent/solvent solution can be altered (e.g. 0.001 mL per sec to 10 mL per sec) to ensure that a good coating of the therapeutic agent is obtained. The coated device can be air-dried. The spray coating process can be repeated one or more times depending on the specific application. The device can be dried under vacuum to reduce residual solvent levels. This process will result in the therapeutic agent being adsorbed into the medical device as well as being surface associated. In one embodiment, the exposure time of the device to the solvent would be such that the device would incur no significant permanent dimensional changes. The therapeutic agent may also be present on the surface of the device. The amount of surface associated therapeutic agent may be reduced by dipping the coated device into a solvent for the therapeutic agent or by spraying the coated device with a solvent for the therapeutic agent.

In one embodiment, the therapeutic agent and a polymer are dissolved in a solvent, for both the polymer and the fibrosis-inhibiting agent, and are then spray coated onto the device.

The solvent may be an inert solvent for the device such that the solvent does not dissolve the medical device to any great extent and is not absorbed by the device to any great extent. The device can be spray coated, either partially or completely, in the therapeutic agent/polymer/solvent solution for a specific period of time. The rate of spraying of the therapeutic agent/solvent solution can be altered (e.g. 0.001 mL per sec to 10 mL per sec) to ensure that a good coating of the therapeutic agent is obtained. The coated device can be air-dried. The spray coating process can be repeated one or more times depending on the specific application. The device can be dried under vacuum to reduce residual solvent levels. This process will result in the therapeutic agent/polymer being coated on the surface of the device.

The solvent may be one that will not dissolve the device but will be absorbed by the device. These solvents can thus swell the device to some extent. The device can be spray coated, either partially or completely, in the therapeutic agent/polymer/solvent solution. The rate of spraying of the therapeutic agent/solvent solution can be altered (e.g., 0.001 mL per sec to 10 mL per sec) to ensure that a good coating of the therapeutic agent is obtained. The coated device can be air-dried. The spray coating process can be repeated one or more times depending on the specific application. The device can be dried under vacuum to reduce residual solvent levels. This process will result in the therapeutic agent/polymer being coated onto the surface of the device as well as the potential for the therapeutic agent being adsorbed into the medical device. The therapeutic agent may also be present on the surface of the device. The amount of surface associated therapeutic agent may be reduced by dipping the coated device into a solvent for the therapeutic agent or by spraying the coated device with a solvent for the therapeutic agent.

The solvent is one that will be absorbed by the device and that will dissolve the device. The device can be spray coated, either partially or completely, in the therapeutic agent/solvent solution. The rate of spraying of the therapeutic agent/solvent solution can be altered (e.g., 0.001 mL per sec to 10 mL per sec) to ensure that a good coating of the therapeutic agent is obtained. The coated device can be air-dried. The spray coating process can be repeated one or more times depending on the specific application. The device can be dried under vacuum to reduce residual solvent levels. In ae preferred embodiment, the exposure time of the device to the solvent would be such that there is not significant permanent dimensional changes to the device (other than those associated with the coating itself). The therapeutic agent may also be present on the surface of the device. The amount of surface associated therapeutic agent may be reduced by dipping the coated device into a solvent for the therapeutic agent or by spraying the coated device with a solvent for the therapeutic agent.

The coating solutions have low viscosities, typically less than 100 CPS, and have good spreading properties. The coatings are preferably baked at elevated temperatures, generally at about 50° C. to about 140° C., to drive off the organic solvents. They could also be dried in a vacuum oven for higher boiling solvents. It may be necessary to treat some surfaces like polyethylene with gas plasma or other ionizing treatment to promote interaction with the coating and adhesion to the substrates.

The coating may contain polymers in addition to the stabilizing polymer such as polyurethane, polyester, styrene polybutadiene, polyvinylidene chloride, polycarbonate, and polyvinyl chloride, preferably in the inner layer to promote adhesion to the surface of the device.

Examples of active agents that can be combined with the hybrid polymer carrier layers of the invention include, in addition to those described elsewhere in this document below, include anti-fibrin and fibrinolytic agents, including plasmin, streptokinase, single chain urokinase, urokinase, t-PA (tissue type plasminogen activator), aminocaproic acid; anti-platelet agents including, aspirin, prostacyclins (and analogues); glycoprotein IIb/IIIa agents including monoclonal antibodies, peptides (e.g. ReoPro, Cilastagel, eptifibatide, tirofiban, ticlopidine, Vapiprost, dipyridamole, forskolin, angiopeptin, argatroban), thromboxane inhibitors; anti-thrombin and anti-coagulant agents, including dextran, heparin, LMW heparin (Enoxaparin, Dalteparin), hirudin, recombinant hirudin, anti-thrombin, synthetic antithrombins, thrombin inhibitors, Warfarin (and other coumarins); anti-mitotic, antiproliferative and cytostatic agents, including vincristine, vinblastine, paclitaxel, methotrexate, cisplatin, fluorouracil, rapamycin, azathioprine, cyclophosphamide, mycophenolic acid, corticosteroids, colchicine, nitroprusside; antiangiogenic and angiostatic agents, including paclitaxel, angiostatin and endostatin; genetic materials and oligonucleotides; ACE inhibitors (e.g. Cilazapril, Lisinopril, Captopril); growth factor (e.g. VEGF, FGF) antagonists; antioxidants and vitamins (e.g. Probucol, Tocopherol); calcium channel blockers (e.g. nifedipine); fish oil (omega 3-fatty acid); phosphodiesterase inhibitors (e.g. dipyridamole); nitric acid donor (e.g. Molsidomine); somatostatin analogues (e.g. angiopeptin); immunosuppressives and anti-inflammatory agents (e.g. prednisolone, glucocorticoid and dexamethasone); antimicrobials (e.g. rifamycin) and radionuclides, including alpha, beta and gamma emitting isotopes (e.g. Re-188, Re-186, 1-125, Y-90); COX-2 inhibitors such as Celecoxib and Vioxx to; kinase inhibitors, such as epidermal growth factor kinase inhibitor, tyrosine kinase inhibitors, MAP kinase inhibitors protein transferase inhibitors, Resten-NG, and other biologically active agents and biologic response modifiers, and others, alone or in combinations to exert multiple actions simultaneously in order to prevent restenosis, and provide other desired biological effects in addition to an antifibrotic effect.

The amount of active agent(s) which may be associated with the device surface using the coatings of the invention is generally in the range of from about 0.05 μg/mm² to about 1 mg/mm², although lower or higher loadings may be used depending on a variety of factors, including the drug, the desired dosage level, the drug release layer composition, the type of anastomotic connector, the diameter and length of anastomotic connector, the number of layers and how the active agent is applied, the coating thickness, the chemical characteristics of the active agent, and other factors. These factors are adjusted to provide a durable coating that controllably releases the desired amount of active agent over an extended period. In a typical desired release pattern, 1-25% of the active agent is released in the first few days, the remainder being released gradually over 30 or more days. Other release patterns may readily be achieved using the inventive methods and compositions, depending on the therapeutic effect desired (e.g., anti-angiogenesis, anti-proliferative, etc.).

The hybrid polymer layers possess physical properties that enable their useful application on anastomotic connector devices. For instance, the hybrid polymers achieve excellent adhesion on metallic anastomotic connector device surfaces. The adhesion of the hybrid polymer layers of the invention is made possible by the use of certain bonding layers as described in U.S. Pat. No. 5,997,517.

Furthermore, the hybrid polymers, together with the multi-layer composite structure, ensure that the drug layers will remain well adhered to the device surface, even during manufacture, sterilization, storage and placement in the patient of the anastomotic connector, and will not lose their adhesion during prolonged implantation. The coatings preferably do not alter the mechanical anastomotic connector functions.

In one embodiment of the invention, the production of anastomotic connectors can begin with the application of the bonding primer layer. In one embodiment, the primer layer can be on the order of about 0.01 microns to about 25 microns thick. Cross-linked primer layers can be thinner than non-cross-linked layers. The primer layer can be applied by dipping the anastomotic connector in the primer coating solution, followed by drying at elevated temperatures in order to drive off the solvents in the coating solution, and when desirable to cure and cross-link the primer layer.

The primer layer may be subjected to turbulent airflow to open any unintended bridging that occurs prior to the curing step. It is also possible to spray the primer coating onto the anastomotic connector. Typical curing schedules include drying for fifteen to sixty minutes at 100° C. to 120° C. The hybrid polymer primer layers comprise polymeric alloys that include such polymers and copolymers as acrylate polymers and copolymers, especially those having functional groups including amine, hydroxyl, and carboxyl, etc., epoxy resins, amine resins, ethylene acrylic acid copolymers, polyurethanes (especially more hydrophobic versions), copolymers of polyvinylpyrrolidone such as with vinyl acetate, polyether sulfones, and others.

In certain embodiments, the use of one or more intermediate layers is optional, although preferred. The intermediate layer can be applied over the primer layer using substantially the same methods as described for the primer layer, including similar curing schedules at elevated temperatures. The intermediate layer is employed to enhance the flexibility, elasticity, and coating uniformity properties of the composite coating layers. It is recognized that thin layers in a composite when constructed appropriately will acquire the properties of its components. The intermediate layer is intended to contribute to and enhance the flexibility, elasticity, and expandability properties of the composite layers. An example of a polymer which performs well in this role is a polycarbonate-polyurethane having a flexural modulus (1% secant modulus (psi) (ASTM procedure D790)) greater than 1,000 or 3,000, and elongation at break greater than 200% or 300%. In a typical embodiment, the primer layer preferably would be about 0.1 to about 5 microns thick, and the intermediate layer would be about 0.01 to about 25 microns thick.

Polymers and copolymers may be used in the intermediate layer for promotion of adhesion, coating uniformity, and flexibility as needed. Such polymers include but are not limited to vinyl acetals, especially polyvinylbutyral, polyurethanes, polycarbonate urethanes, and acrylate polymers and copolymers.

The drug releasing hybrid polymer layer can comprise two or more polymers, together with one or more drugs, which can be dissolved in an organic solvent or solvent mixture. The drug(s) are usually dissolved in the organic solvent mixture, but may also be present as dispersions of solid particles. The hybrid polymer matrix forms a polymeric alloy upon drying. In the preferred embodiment, this layer can be typically about 1 micron to about 10 microns thick. The hybrid polymer matrix can be applied as one layer, or as two or more layers, and different drugs may be present in the same or different layer(s). When multiple layers are employed, the different layers could have the same or different drug release properties.

Soluble drugs can also form into the polymeric alloy at the molecular level. An organic solvent or solvent mixture can be selected so that it is a mutual solvent for the polymeric and soluble drug components, while in the liquid form, and throughout the drying process. It is also preferable if the solvent has the ability to swell the substrate, thereby enabling some of the drug-hybrid polymer components to penetrate superficially into the substrate surface and gain improved adhesion. The polymeric components of the drug releasing layer can comprise cellulose esters to stabilize and preserve the drug components, and usually contain a polyurethane. The polyurethane contributes flexibility, adhesion promotion, elasticity, and expandability to the drug-releasing layer. Other polymers may also be incorporated into the layer, including hydrophilic, water soluble polymers such polyvinylpyrrolidone (PVP), PVP copolymers, polyethylene glycol, polyethylene oxide water soluble cellulose ethers and esters such hydroxymethylcellulose, others. Drugs selected from the groups that were previously cited may be incorporated, alone or in combinations.

In one embodiment of the invention, the coating solutions are prepared by first dissolving the polymer components in the solvent mixtures. It is also possible to dissolve the individual polymer components separately in solutions, and then to combine together separate solutions of the individual polymers. The drug(s) are then usually incorporated into the hybrid polymer solution, although the drugs can be added before the polymers. The drug releasing coating is then applied over the anastomotic connector, which already has one, or more polymer coatings, using the same methods as used for the other polymer coatings. After coating, the coating is dried for five to sixty minutes at temperatures of about 40° C. to about 120° C.

The coated anastomotic connectors can be packaged and sterilized. Ethylene oxide is useful for sterilization of anastomotic connectors prepared according to the invention.

As described above, the coatings of the invention may include a primer (bonding) layer and, optionally, an intermediate layer. The primer layer can be formed from a combination of polymers, such as an acrylate/carboxyl polymer, an epoxy polymer, and a polyvinylpyrrolidone vinylacetate copolymer (PVPNA) or ethylene acrylic acid copolymer (EM), an epoxy polymer, and a polycarbonate urethane. Other polymers that may be used in the primer layer include, e.g., polyimide copolymers, polyamide copolymers, polyether sulfone polymers, polyethylene glycol polymers, polyethylene oxide polymers, and other polymers which typically are used in metal primer applications. The intermediate layer may include, e.g., polycarbonate polyurethane, flexible acrylate polymers/copolymers including butyl acrylate, polyvinyl butyral, or other elastic polymers used alone or in hybrid polymer combinations.

In one embodiment, a drug release layer polymer combination suitable for use with the invention is acrylate/carboxyl polymer+epoxy polymer+polyvinylpyrrolidone vinylacetate copolymer (PVPNA). Another combination includes RS nitrocellulose plus any of the following: polytetramethylene ether glycol urethane, polycarbonate-urethanes, PVP, polyethylene glycol, polyethylene oxide, methylvinylether maleic anhydride copolymer, and/or poly(2-hydroxyethyl methacrylate).

Active ingredients that may be used in combination with any of the coatings describe above include agents from any of the classes described above, such as, for example, paclitaxel, doxorubicin, mycophenolic acid, benzalkonium heparinate, rifamycin, and methotrexate, 5-FU, tacrolimus and other agents).

As anastomotic connector devices are made in a variety of configurations and sizes, the exact dose administered will vary with device size, surface area and design. However, certain principles can be applied in the application of this art. Drug dose can be calculated as a function of dose per unit area (of the portion of the device being coated), total drug dose administered can be measured, and appropriate surface concentrations of active drug can be determined. Regardless of the method of application of the drug to the anastomotic connector device, the preferred therapeutic agents, used alone or in combination, should be administered under the following application and dosing guidelines:

Within certain embodiments of the invention, application of the therapeutic agent can be through direct deposition onto all or a portion of the device (see, e.g., U.S. Pat. Nos. 6,096,070 and 6,299,604), and/or admixed with a delivery system or carrier (e.g., a polymer, liposome, or vitamin as discussed above) which is applied to all or a portion of the device (see the patents, patent applications, and references listed above under “Compositions and Formulations.”

Within certain aspects of the invention, therapeutic agents can be attached to an anastomotic device using non-covalent attachments. For example, for compounds that are relatively sparingly water soluble or water insoluble, the compound can be dissolved in an organic solvent at a specified concentration. The solvent chosen for this application would not result in dissolution or swelling of the polymeric device surface. The anastomotic connector device can then be dipped into the solution, withdrawn and then dried (air dried and/or vacuum dried). Alternatively, the drug solution can be sprayed onto the surface of the device using current spray coating technology. Typically, drug release from the anastomotic connector device coated in this manner would be of a relatively short duration and would be a function of the solubility of the drug in the body fluid into which it was placed (most commonly blood for the endoluminal portion of the device and extracellular fluid for the adventitial portion of the device) and the degree of fluid diffusivity into the polymer.

In another aspect, the therapeutic agent(s) can be dissolved in a solvent that has the ability to swell or partially dissolve the surface of a constituent polymer used in the manufacturing of the anastomotic device. Depending on the solvent/device polymer combination, the device can be dipped into the drug solution for a period of time such that the drug can diffuse into the surface layer of the polymeric portion of the device. Alternatively the drug solution can be sprayed onto all or a part of the surface of the device. The release profile of the drug depends upon the solubility of the drug in the surface polymeric layer. Using this approach, one would ensure that the solvent does not result in a significant distortion or dimensional change of the anastomotic connector device.

If the device, or portions of the device, are composed of materials (e.g., stainless steel, nitinol) that do not allow incorporation of the therapeutic agent(s) into the surface layer using the above solvent method, the surface of the device can be treated with a plasma polymerization method such that a thin polymeric layer is deposited onto the device surface. Examples of such methods include parylene coating of devices, and the use of various monomers such hydrocyclosiloxane monomers. A parylene primer layer may be deposited onto the anastomotic connector device using a parylene coater (e.g., PDS 2010 LABCOTER2 from Cookson Electronics) and a suitable reagent (e.g., di-p-xylylene or dichloro-di-p-xylylene) as the coating feed material. Parylene compounds are commercially available, for example, from Specialty Coating Systems, Indianapolis, Ind.), including Parylene N (di-p-xylylene), Parylene C (a monchlorinated derivative of Parylene N, and Parylene D, a dichlorinated derivative of Parylene N).

The dip coating or spray coating methods described above then may be used to incorporate the therapeutic agent(s) into the coated surface of the device.

For therapeutic agents that have some degree of water solubility, the retention of these compounds onto the anastomotic device is relatively short-lived. For therapeutic agents that contain ionic groups, it is possible to ionically complex these agents to oppositely charged compounds that have a hydrophobic component. For example therapeutic agents containing amine groups can be complexed with compounds such as sodium dodecyl sulfate (SDS). Compounds containing carboxylic groups can be complexed with tridodecymethyammonium chloride (TDMAC). Mitoxantrone, for example has two secondary amine groups and comes as a chloride salt. This compound can be added to sodium dodecyl sulfate in order to form a complex. This complex can be dissolved in an organic solvent which can then be dip coated or spray coated. Doxorubicin has an amine group and could thus also be complexed with SDS. This complex can then be applied to the device by dip coating or spray coating methods.

Therapeutic agents with available functional groups can be covalently attached to the anastomotic connector device surface using several chemical methods. If the polymeric material used to manufacture the device has available surface functional groups then these can be used for covalent attachment of the agent. For example, if the device surface contains carboxylic acid groups, these groups can be converted to activated carboxylic acid groups (e.g., acid chlorides, succinimidyl derivatives, 4-nitrophenyl ester derivatives etc.). These activated carboxylic acid groups can then be reacted with amine functional groups that are present on the therapeutic agent (e.g., mitoxantrone).

For surfaces that do not contain appropriate functional groups, these groups can be introduced to the polymer surface via a plasma treatment regime. For example, carboxylic acid groups can be introduced via a plasma treatment process (e.g., the use of O₂ and/or CO₂ as a component in the feed gas mixture). The carboxylic acid groups can also be introduced using acrylic acid or methacrylic acid in the gas stream. These carboxylic acid groups can then be converted to activated carboxylic acid groups (e.g., acid chlorides, succinimidyl derivatives, 4-nitrophenyl ester derivatives etc) that can subsequently be reacted with amine functional groups that are present on the therapeutic agent.

In certain aspects, the drug-containing layer may be coated with a surface layer that may serve to protect the drug-releasing layer and/or provide a means of delaying release of the drug. For example, in one aspect, the device is coated with a primer layer (e.g., a parylene coating) and then coated with a solution of drug (e.g., paclitaxel in a solvent). The solvent then is removed. The coated device then is coated with a surface coating containing, for example, parylene.

In certain aspects of the invention, the therapeutic agent or therapeutic agent/carrier coating can be further coated with another layer that will act to modulate the release of the therapeutic agent or the surface properties of the therapeutic agent/carrier layer. For example, the therapeutic agent/carrier layer could be coated with a coating that results in a lubricious surface. Polymers that are useful in this aspect include poly(vinyl pyrrolidone), poly(vinyl pyrrolidone-co-vinylacetate), poly(vinyl alcohol), poly(ethylene glycol) and poly(ethylene oxide). For example, a therapeutic agent/carrier layer may be coated with a surface coating in which the therapeutic agent is less soluble compared to the carrier. Alternatively, a therapeutic agent/carrier layer may be coated with a surface coating in which the therapeutic agent is more soluble compared to the carrier.

In another aspect of the invention, the coated device can be subjected to a treatment that increases the crosslink density at the surface of the coated areas. This can be accomplished by subjecting the coated device to a plasma treatment.

With respect to dosing of the desired therapeutic agent(s) on the anastomotic connector device, total dosage delivered, drug dosage as a function of device surface area and the duration of drug delivery will be dependent, at least in part, on the solubility of the compound and whether the drug is administered on the endoluminal surface of the device, the intramural (i.e., within the vessel wall) portion of the device, the adventitial surface of the device, or a combination of these. In general, for water insoluble drugs, lower drug doses and shorter durations of drug delivery are used when the drug is delivered from the endoluminal surface of the anastomotic connector device since the compound tends to move into the blood vessel wall down both a concentration and pressure gradient. For water soluble compounds, higher doses and more sustained delivery may be used as the drug will have a tendency to be “washed off” the endoluminal surface and taken away in the aqueous bloodstream. Conversely, the opposite tends to be true for water insoluble drugs administered to the adventitial surface of the anastomotic connector device—higher drug doses and longer durations of drug delivery may be used since the compound must move into the blood vessel wall against both a hydrostatic and pressure gradient. For water soluble compounds, higher doses and more sustained delivery may also be used, as the drug will have a tendency to dissipate into the tissue fluid (and away from the vessel wall) and must also move into the blood vessel wall against both a hydrostatic and pressure gradient. Drug administered into the blood vessel wall behaves as a hybrid between the two, usually requiring lower doses and shorter delivery times than adventitially delivered drug for both soluble and insoluble therapeutic agents.

As described previously and in subsequent sections, anastomotic connector devices are produced in a variety of designs. Some designs contain endoluminal surface segments only, some contain intramural segments, some contain adventitial segments only, and many contain segments of the device in all three anatomical areas.

The following is a description of examples of anti-fibrotic agents and dosing ranges. It should be readily evident that any of the drugs and analogues and derivatives described herein can be utilized in the practice of the invention. Drug dosing for exemplary therapeutic agents may vary depending upon whether the drug is released from an endoluminal (intravascular) surface, an intramural (within the arterial wall) surface and/or an adventitial (outer vessel wall) surface. These examples are provided by way of explanation and not by way of limitation. The dosing parameters described below are adjusted based on the relative potency of the drug and/or its analogue or derivative (e.g., a compound twice as potent as a drug described below is administered at half the above parameters, a compound half as potent as a drug mentioned below is administered at twice the above parameters, etc.).

(a) Anthracyclines. Utilizing the anthracycline doxorubicin as an example, whether contained within a polymer coating, incorporated into the polymers which make up the device, or applied without use of a polymer, the total dose of doxorubicin applied to the anastomotic connector device (and the other components of the anastomosis) should not exceed 25 mg (range of 0.1 μg to 25 mg). In one embodiment, the total amount of drug applied to the anastomotic connector device (and the other components of the anastomosis) should be in the range of 1 μg to 10 mg. The dose per unit area of the device (i.e., the amount of drug as a function of the surface area of the portion of the device to which drug is applied and/or incorporated) should fall within the range of about 0.01 μg to about 100 μg per mm² of surface area. In a preferred embodiment, doxorubicin should be applied to the device surface at a dose of about 0.1 μg/mm² to about 10 μg/mm². As different polymeric and non-polymeric coatings will release doxorubicin at differing rates, the above dosing parameters should be utilized in combination with the release rate of the drug from the device surface such that a minimum concentration of about 10⁻⁷ to about 10⁻⁴ M doxorubicin is maintained on the device surface. In a further embodiment, doxorubicin is released from the surface of the device such that inhibitory activity is maintained for a period ranging from several hours to several months. In a particularly preferred embodiment the drug is released in effective concentrations for a period ranging from about 1 day to about 90 days. It should be readily evident given the discussions provided herein that analogues and derivatives of doxorubicin (as described previously) with similar functional activity can be utilized for the purposes of this invention; the above dosing parameters are then adjusted of the relative potency of the analogue or derivative as compared to the parent compound (e.g., a compound twice as potent as doxorubicin is administered at half the above parameters, a compound half as potent as doxorubicin is administered at twice the above parameters).

Utilizing mitoxantrone, as another example of an anthracycline, whether applied contained within a polymer coating, incorporated into the polymers which make up the device, or applied without a carrier polymer, the total dose of mitoxantrone applied to the anastomotic connector device (and the other components of the anastomosis) should not exceed 5 mg (range of about 0.01 μg to about 5 mg). In one preferred embodiment, the total amount of drug applied to the anastomotic connector device (and the other components of the anastomosis) should be in the range of about 0.1 μg to about 1 mg. The dose per unit area of the device (i.e., the amount of drug as a function of the surface area of the portion of the device to which drug is applied and/or incorporated) should fall within the range of about 0.01 μg to about 20 μg per mm² of surface area. In a preferred embodiment, mitoxantrone should be applied to the device surface at a dose of about 0.05 μg/mm² to about 10 μg/mm². As different polymeric and non-polymeric coatings will release mitoxantrone at differing rates, the above dosing parameters should be utilized in combination with the release rate of the drug from the device surface such that a minimum concentration of about 10⁻⁵to about 10⁻⁸ M of mitoxantrone is maintained on the device surface. In a preferred embodiment, mitoxantrone is released from the surface of the device such that inhibitory activity is maintained for a period ranging from several hours to several months. In a further preferred embodiment the drug is released in effective concentrations for a period ranging from about 1 day to about 90 days. It should be readily evident given the discussions provided herein that analogues and derivatives of mitoxantrone (as described previously) with similar functional activity can be utilized for the purposes of this invention; the above dosing parameters are then adjusted of the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Taxanes. Utilizing the taxane paclitaxel as an example, whether applied contained within a polymer coating, incorporated into the polymers which make up the device, or applied without use of a polymer, the total dose of paclitaxel applied to the anastomotic connector device (and the other components of the anastomosis) should not exceed 25 mg (range of about 0.1 μg to about 25 mg). In one embodiment, the total amount of drug applied to the anastomotic connector device (and the other components of the anastomosis) should be in the range of about 1 μg to about 10 mg. The dose per unit area of the device (i.e., the amount of drug as a function of the surface area of the portion of the device to which drug is applied and/or incorporated) should fall within the range of about 0.1 μg to about 10 μg per mm² of surface area. In a preferred embodiment, paclitaxel should be applied to the device surface at a dose of about 0.25 μg/mm² to about 5 μg/mm². As different polymer and non-polymer coatings will release paclitaxel at differing rates, the above dosing parameters should be utilized in combination with the release rate of the drug from the device surface such that a minimum concentration of about 10⁻⁸to about 10⁻⁴ M of paclitaxel is maintained on the device surface. In a preferred embodiment, paclitaxel is released from the surface of the device such that inhibitory activity is maintained for a period ranging from several hours to several months. In a further embodiment the drug is released in effective concentrations for a period ranging from about 1 to about 90 days. It should be readily evident given the discussions provided herein that analogues and derivatives of paclitaxel (such as docetaxel and others described previously) with similar functional activity can be utilized for the purposes of this invention; the above dosing parameters are then adjusted of the relative potency of the analogue or derivative as compared to the parent compound (e.g., a compound twice as potent as paclitaxel is administered at half the above parameters, a compound half as potent as paclitaxel is administered at twice the above parameters, etc.).

(c) Immunosuppressants. Utilizing the immunosuppressant sirolimus (also known as Rapamycin, Rapamune) as an example, whether applied contained within a polymer coating, incorporated into the polymers which make up the device, or applied without use of a polymer, the total dose of sirolimus applied to the anastomotic connector device (and the other components of the anastomosis) should not exceed 10 mg (range of about 0.1 μg to about 10 mg). In one preferred embodiment, the total amount of drug applied to the anastomotic connector device (and the other components of the anastomosis) should be in the range of about 1 μg to about 10 mg. The dose per unit area of the device (i.e., the amount of drug as a function of the surface area of the portion of the device to which drug is applied and/or incorporated) should fall within the range of about 0.1 μg to about 100 μg per mm² of surface area. In another embodiment, sirolimus should be applied to the device surface at a dose of about 0.5 μg/mm² to about 10 μg/mm². As different polymer and non-polymer coatings will release sirolimus at differing rates, the above dosing parameters should be utilized in combination with the release rate of the drug from the device surface such that a minimum concentration of about 10⁻⁸ to about 10⁻⁴ M of sirolimus is maintained on the device surface. In a further embodiment, sirolimus is released from the surface of the device such that inhibitory activity is maintained for a period ranging from several hours to several months. In a particularly preferred embodiment the drug is released in effective concentrations for a period ranging from about 1 to about 90 days.

Utilizing the immunosuppressant everolimus as an example, whether applied contained within a polymer coating, incorporated into the polymers which make up the device, or applied without use of a polymer, the total dose of everolimus applied to the anastomotic connector device (and the other components of the anastomosis) should not exceed 10 mg (range of about 0.1 μg to about 10 mg). In one preferred embodiment, the total amount of drug applied to the anastomotic connector device (and the other components of the anastomosis) should be in the range of about 10 μg to about 1 mg. The dose per unit area of the device (i.e., the amount of drug as a function of the surface area of the portion of the device to which drug is applied and/or incorporated) should fall within the range of about 0.1 μg to about 100 μg per mm² of surface area. In a preferred embodiment, everolimus should be applied to the device surface at a dose of about 0.3 μg/mm² to about 10 μg/mm². As different polymer and non-polymer coatings will release everolimus at differing rates, the above dosing parameters should be utilized in combination with the release rate of the drug from the device surface such that a minimum concentration of about 10⁻⁸to about 10⁻⁴ M everolimus is maintained on the device surface. In a further embodiment, everolimus is released from the surface of the device such that inhibitory activity is maintained for a period ranging from several hours to several months. In a particularly preferred embodiment the drug is released in effective concentrations for a period ranging from about 1 day to about 90 days.

(d) Topoisomerase Inhibitors. Utilizing the topoisomerase inhibitor camptothecin as an example, whether applied contained within a polymer coating, incorporated into the polymers which make up the device, or applied without use of a polymer, the total dose of camptothecin applied to the anastomotic connector device (and the other components of the anastomosis) should not exceed 25 mg (range of about 0.1 μg to about 25 mg). In one embodiment, the total amount of drug applied to the anastomotic connector device (and the other components of the anastomosis) should be in the range of about 1 μg to about 10 mg. The dose per unit area of the device (i.e., the amount of drug as a function of the surface area of the portion of the device to which drug is applied and/or incorporated) should fall within the range of about 0.1 μg to about 10 μg per mm² of surface area. In a preferred embodiment, camptothecin should be applied to the device surface at a dose of about 0.25 μg/mm² to about 5 μg/mm². As different polymer and non-polymer coatings will release camptothecin at differing rates, the above dosing parameters should be utilized in combination with the release rate of the drug from the device surface such that a minimum concentration of about 10⁻⁸ to about 10⁻⁴ M of camptothecin is maintained on the device surface. In a preferred embodiment, camptothecin is released from the surface of the device such that inhibitory activity is maintained for a period ranging from several hours to several months. In a further embodiment the drug is released in effective concentrations for a period ranging from about 1 day to about 90 days. It should be readily evident given the discussions provided herein that analogues and derivatives of camptothecin with similar functional activity can be utilized for the purposes of this invention; the above dosing parameters are then adjusted of the relative potency of the analogue or derivative as compared to the parent compound (e.g., a compound twice as potent as camptothecin is administered at half the above parameters, a compound half as potent as camptothecin is administered at twice the above parameters, etc.).

(e) IMPDH Inhibitors. Utilizing the IMPDH inhibitor mycophenolic acid as an example, whether applied contained within a polymer coating, incorporated into the polymers which make up the device, or applied without use of a polymer, the total dose of mycophenolic acid applied to the anastomotic connector device (and the other components of the anastomosis) should not exceed 100 mg (range of about 0.1 μg to about 100 mg). In one embodiment, the total amount of drug applied to the anastomotic connector device (and the other components of the anastomosis) should be in the range of about 1 μg to about 50 mg. The dose per unit area of the device (i.e., the amount of drug as a function of the surface area of the portion of the device to which drug is applied and/or incorporated) should fall within the range of about 0.1 μg to about 50 μg per mm² of surface area. In a preferred embodiment, mycophenolic acid should be applied to the device surface at a dose of about 0.25 μg/mm² to about 25 μg/mm². As different polymer and non-polymer coatings will release mycophenolic acid at differing rates, the above dosing parameters should be utilized in combination with the release rate of the drug from the device surface such that a minimum concentration of about 10⁻⁸ to about 10⁻³ M of mycophenolic acid is maintained on the device surface. In a preferred embodiment, mycophenolic acid is released from the surface of the device such that inhibitory activity is maintained for a period ranging from several hours to several months. In a further embodiment the drug is released in effective concentrations for a period ranging from about 1 day to about 90 days. It should be readily evident given the discussions provided herein that analogues and derivatives of mycophenolic acid with similar functional activity can be utilized for the purposes of this invention; the above dosing parameters are then adjusted of the relative potency of the analogue or derivative as compared to the parent compound (e.g., a compound twice as potent as mycophenolic acid is administered at half the above parameters, a compound half as potent as mycophenolic acid is administered at twice the above parameters, etc.).

(f) Podophyllotoxins. Utilizing the Podophyllotoxin etoposide as an example, whether applied contained within a polymer coating, incorporated into the polymers which make up the device, or applied without use of a polymer, the total dose of etoposide applied to the anastomotic connector device (and the other components of the anastomosis) should not exceed 25 mg (range of about 0.1 μg to about 25 mg). In one embodiment, the total amount of drug applied to the anastomotic connector device (and the other components of the anastomosis) should be in the range of about 1 μg to about 10 mg. The dose per unit area of the device (i.e., the amount of drug as a function of the surface area of the portion of the device to which drug is applied and/or incorporated) should fall within the range of about 0.1 μg to about 10 μg per mm² of surface area. In a preferred embodiment, etoposide should be applied to the device surface at a dose of about 0.25 μg/mm² to about 5 μg/mm². As different polymer and non-polymer coatings will release etoposide at differing rates, the above dosing parameters should be utilized in combination with the release rate of the drug from the device surface such that a minimum concentration of about 10⁻⁸tp about 10⁻⁴ M of etoposide is maintained on the device surface. In a preferred embodiment, etoposide is released from the surface of the device such that inhibitory activity is maintained for a period ranging from several hours to several months. In a further embodiment the drug is released in effective concentrations for a period ranging from about 1 day to about 90 days. It should be readily evident given the discussions provided herein that analogues and derivatives of etoposide with similar functional activity can be utilized for the purposes of this invention; the above dosing parameters are then adjusted of the relative potency of the analogue or derivative as compared to the parent compound (e.g., a compound twice as potent as etoposide is administered at half the above parameters, a compound half as potent as etoposide is administered at twice the above parameters, etc.).

(g) HSP 90 Antagonists. Utilizing the HSP 90 Antagonist geldanamycin as an example, whether applied contained within a polymer coating, incorporated into the polymers which make up the device, or applied without use of a polymer, the total dose of geldanamycin applied to the anastomotic connector device (and the other components of the anastomosis) should not exceed 25 mg (range of about 0.1 μg to about 25 mg). In one embodiment, the total amount of drug applied to the anastomotic connector device (and the other components of the anastomosis) should be in the range of about 1 μg to about 10 mg. The dose per unit area of the device (i.e., the amount of drug as a function of the surface area of the portion of the device to which drug is applied and/or incorporated) should fall within the range of about 0.1 μg to about 10 μg per mm² of surface area. In a preferred embodiment, geldanamycin should be applied to the device surface at a dose of about 0.25 μg/mm² to about 5 μg/mm². As different polymer and non-polymer coatings will release geldanamycin at differing rates, the above dosing parameters should be utilized in combination with the release rate of the drug from the device surface such that a minimum concentration of about 10⁻⁸ to about 10⁻⁴ M of geldanamycin is maintained on the device surface. In a preferred embodiment, geldanamycin is released from the surface of the device such that inhibitory activity is maintained for a period ranging from several hours to several months. In a further embodiment the drug is released in effective concentrations for a period ranging from about 1 day to about 90 days. It should be readily evident given the discussions provided herein that analogues and derivatives of geldanamycin with similar functional activity can be utilized for the purposes of this invention; the above dosing parameters are then adjusted of the relative potency of the analogue or derivative as compared to the parent compound (e.g., a compound twice as potent as geldanamycin is administered at half the above parameters, a compound half as potent as geldanamycin is administered at twice the above parameters, etc.).

(h) Pyrolidine antibiotics. Utilizing the pyrolidine antibiotic anisomycin as an example, whether applied contained within a polymer coating, incorporated into the polymers which make up the device, or applied without use of a polymer, the total dose of anisomycin applied to the anastomotic connector device (and the other components of the anastomosis) should not exceed 25 mg (range of about 0.1 μg to about 25 mg). In one embodiment, the total amount of drug applied to the anastomotic connector device (and the other components of the anastomosis) should be in the range of about 1 μg to about 10 mg. The dose per unit area of the device (i.e., the amount of drug as a function of the surface area of the portion of the device to which drug is applied and/or incorporated) should fall within the range of about 0.1 μg to about 10 μg per mm² of surface area. In a preferred embodiment, anisomycin should be applied to the device surface at a dose of about 0.25 μg/mm² to about 5 μg/mm². As different polymer and non-polymer coatings will release anisomycin at differing rates, the above dosing parameters should be utilized in combination with the release rate of the drug from the device surface such that a minimum concentration of about 10⁻⁸ to about 10⁻⁴ M of anisomycin is maintained on the device surface. In a preferred embodiment, anisomycin is released from the surface of the device such that inhibitory activity is maintained for a period ranging from several hours to several months. In a further embodiment the drug is released in effective concentrations for a period ranging from about 1 day to about 90 days. It should be readily evident given the discussions provided herein that analogues and derivatives of anisomycin with similar functional activity can be utilized for the purposes of this invention; the above dosing parameters are then adjusted of the relative potency of the analogue or derivative as compared to the parent compound (e.g., a compound twice as potent as anisomycin is administered at half the above parameters, a compound half as potent as anisomycin is administered at twice the above parameters, etc.).

(i) Angiogenesis Inhibitors. Utilizing the angiogenesis inhibitor halofuginone as an example, whether applied contained within a polymer coating, incorporated into the polymers which make up the device, or applied without use of a polymer, the total dose of halofuginone applied to the anastomotic connector device (and the other components of the anastomosis) should not exceed 25 mg (range of about 0.1 μg to about 25 mg). In one embodiment, the total amount of drug applied to the anastomotic connector device (and the other components of the anastomosis) should be in the range of about 1 μg to about 10 mg. The dose per unit area of the device (i.e., the amount of drug as a function of the surface area of the portion of the device to which drug is applied and/or incorporated) should fall within the range of about 0.1 μg to about 10 μg per mm² of surface area. In a preferred embodiment, halofuginone should be applied to the device surface at a dose of about 0.25 μg/mm² to about 5 μg/mm². As different polymer and non-polymer coatings will release halofuginone at differing rates, the above dosing parameters should be utilized in combination with the release rate of the drug from the device surface such that a minimum concentration of about 10⁻⁸ to about 10⁻⁴ M of halofuginone is maintained on the device surface. In a preferred embodiment, halofuginone is released from the surface of the device such that inhibitory activity is maintained for a period ranging from several hours to several months. In a further embodiment the drug is released in effective concentrations for a period ranging from about 1 day to about 90 days. It should be readily evident given the discussions provided herein that analogues and derivatives of halofuginone with similar functional activity can be utilized for the purposes of this invention; the above dosing parameters are then adjusted of the relative potency of the analogue or derivative as compared to the parent compound (e.g., a compound twice as potent as halofuginone is administered at half the above parameters, a compound half as potent as halofuginone is administered at twice the above parameters, etc.).

It should be readily evident given the discussions provided herein that analogues and derivatives of sirolimus (such as those described previously) and related immunosuppressant compounds with similar functional activity such as everolimus (and analogues and derivatives thereof described previously) and tacrolimus (also known as FK506 and analogues and derivatives thereof described previously) can be utilized for the purposes of this invention; the above dosing parameters are then adjusted of the relative potency of the analogue or derivative as compared to the parent compound (e.g., a compound twice as potent as sirolimus is administered at half the above parameters, a compound half as potent as sirolimus is administered at twice the above parameters, etc.).

(j) In one aspect of the invention, the anastomotic connector device comprises less than 25 mg of therapeutic agent, while in another aspect the device comprises an amount of therapeutic agent in the range of about 0.1 μg to about 25 mg. In another aspect, the total amount of drug associated with the anastomotic connector device (and the other components of the anastomosis) is in the range of about 1 μg to about 10 mg. The dose per unit area of the device (i.e., the amount of drug as a function of the surface area of the portion of the device to which drug is applied and/or incorporated) is within the range of about 0.1 μg to about 10 μg per mm² of surface area. In another aspect, the drug is applied to the surface of the device at a dose of about 0.25 μg/mm² to about 5 μg/mm². In one aspect, the device releases drug at a rate such that a minimum concentration of about 10⁻⁸ to about 10⁻⁴ M of drug is maintained on the device surface. In a preferred embodiment, therapeutic agent is released from the surface of the device such that inhibitory activity is maintained for a period ranging from several hours to several months. In a further embodiment the drug is released in effective concentrations for a period ranging from about 1 day to about 90 days.

(d) Combination therapy. It should be readily evident based upon the discussions provided herein that anastomotic connector devices coated with a combination of anthracyclines (e.g., doxorubicin or mitoxantrone), taxanes (e.g., paclitaxel, docetaxel), and/or immunosuppressants (e.g., sirolimus, everolimus, tacrolimus) or other aforementioned agents can be utilized to inhibit anastomotic stenosis/restenosis.

In addition, since thrombogenicity of the anastomosis is associated with an increased risk of stenosis/restenosis, combinations of anthracyclines (e.g., doxorubicin or mitoxantrone), taxanes (e.g., paclitaxel, docetaxel), and immunosuppressants (e.g., sirolimus, everolimus, tacrolimus) or other aforementioned agents can be combined with anti-thrombotic and/or antiplatelet agents (for example, heparin, heparin fragments, dextran sulphate, danaparoid, lepirudin, hirudin, AMP, adenosine, 2-chloroadenosine, aspirin, phenylbutazone, indomethacin, meclofenamate, hydrochloroquine, dipyridamole, iloprost, ticlopidine, clopidogrel, abcixamab, eptifibatide, tirofiban, streptokinase, and/or tissue plasminogen activator) to enhance efficacy. Alternatively, the anti-thrombogenic agent and/or anti-platelet agent can be released from the anastomotic connector device in a different location from the anthracycline, taxane, immunosuppressant, podophyllotoxin,or other aforementioned agent or as a separate layer (e.g., on top of, or beneath this layer).

B. Illustrative Embodiments of Anastomotic Connector Devices Which Release a Desired Therapeutic Agent

A variety of anastomotic connector devices are suitable for use in this invention. All of these devices may be combined with the therapeutic agents described above despite being constructed in a wide array of configurations. Despite the numerous permutations and combinations possible, whether incorporating into or coated onto a portion of the device that resides within the lumen of the vessel (i.e., the luminal (inner) surface of the device), the intramural segment (i.e., the portion of the device which traverses the vessel wall) and/or coating onto or incorporating into the adventitial surface (i.e., the portion of the device contacting the outer (adventitial) surface of the vessel) of the anastomotic connector device, the total dose, dose/unit area and drug surface concentrations remain within the specifications detailed above.

Anastomotic connector devices may be made from a variety of materials, polymeric, metallic, and ceramic materials. Representative examples of polymeric materials that may be used in the manufacture of anastomotic connectors or components of the anastomotic connectors include, for example, polyethylene, polypropylene, polyamides (e.g., nylon and polyether-block co-polyamide polymers sold under the tradename PEBAX, available from Atofina (Philiadelphia, Pa.)), polyesters, polyurethanes, poly(vinyl chloride), silicones, polycarbonate, polysulfone, epoxies, fluoropolymers (e.g., hompolymers and copolymers of hexafluoropropylene, vinylidene fluoride, and tetrafluoroethylene, such as poly(tetrafluoroethylene), available under the tradename TEFLON from E.l. Du Pont De Nemours and Company (Wilmington, Del.), fluorinated ethylene propylene (FEP), polyarylene-etherketone, polyarylene ethers, polyimides, poly(vinylchloride), polyoxymethylene, and PEEK (poly(phenyl ether ether ketone)) or poly(arylene ether ether ketone)

In certain aspects, anastomotic connectors include an bioabsorbable material, such as collagen, polycaprolactone, poly(glycolic acid), poly(lactic acid), poly(3-hydroxybutric acid), polymers and copolymers of lactide and glycolide, and other poly(hydroxyl acids) and polyesterswhere the polyester can comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, Γ-decanolactone, δ-decanolactone, trimethylene carbonate, 1,4-dioxane-2-one or 1,5-dioxepan-2one.

Anastomotic connectors may include one or more types of metals or metal alloys, such as, for example, stainless steel, platinum, gold, nickel, titanium, tungsten, nickel-titanium (NiTi) alloys such as nitinol, chromium, zirconium, aluminum, hafnium, iridium, niobium, palladium, platinum, cobalt chromium, cobalt-chromium-molybdenum alloys, cobalt-chromium-tungsten alloys, tantalum and titanium-aluminum intermetallic alloys.

Anastomotic connector devices may comprise a crystalline or amorphous ceramic material, such as aluminum oxide (alumina), titanium oxide, titanium dioxide (titania), yttrium oxide (yttria), and zirconium oxide (zirconia), silicon dioxide (silica), and compounds based on these and doped with other elements. Other types of ceramics include niobium silicide, niobium oxide, tantalum silicide, tantalum oxide, titanium silicide, tungsten silicide, tungsten oxide, vanadium silicide, zirconium silicide, barium oxide, calcium oxide, hafnium oxide, chromium nitride, iridium oxide, dahlite, brushite, tricalcium phosphate, hydroxyapatite, calcium sulphate, calcium carbonate, silicides, barium titantate, strontium titanate (see, e.g., U.S. Pat. Nos. 6,716,444 and 6,663,662).

Anastomotic connector devices may include a carbonaceous material, such as, pyrolitic carbon, graphite, furnace black, diamond, activated charcoal, carbon black, fumed carbon, gas black, or channel black, or carbyne. Other forms of carbon-containing materials include polymeric carbon films (see, e.g., U.S. Pat. No. 6,454,797) and diamond like carbon (DLC) films (see, e.g., U.S. Pat. Nos. 6,726,718 and 6,379,383), which have been shown to delay clotting time (see, e.g., “Haemocompatibility Evaluation of DLC and SiC coated surfaces” Nurdin N. , Francois P., Mugnier Y., Krumeich J., More. M, Aronson B-O, Descouts P. European Cells and Materials Vol 5, 2003 (pp 17-28)).

Anastomotic connector devices also may be made from a combination of materials (e.g., a metal and a polymeric material) or a composite material, such as a composite of a metal or metal alloy and a ceramic material; a metal or metal alloy and a polymeric material; or a ceramic material and a polymeric material.

Representative examples of anastomotic connector devices include, without limitation, vascular clips, vascular sutures, vascular staples, vascular clamps, suturing devices, anastomotic coupling devices (i.e., anastomotic couplers), including couplers that include tubular segments for carrying blood, and anastomotic rings.

Broadly, anastomotic connector devices may be classified into three categories: (1) automated and modified suturing methods and devices, (2) micromechanical devices, and (3) anastomotic coupling devices.

(1) Automated and Modified Suturing Methods and Devices

Automated sutures and modified suturing methods generally facilitate the rapid deployment of multiple sutures, usually in a single step, and eliminate the need for knot tying or the use of aortic side-biting clamps. Suturing devices include those devices that are adapted to be minimally invasive such that anastomoses are formed between vascular conduits and hollow organ structures by applying sutures or other surgical fasteners through device ports or other small openings. With these devices, sutures and other fasteners are applied in a relatively quick and automated manner within bodily areas that have limited access. By using minimally invasive means for establishing anastomoses, there is less blood loss and there is no need to temporarily stop the flow of blood distal to the operating site. For example, the suturing device may be composed of a shaft-supported vascular conduit that is adapted for anastomosis and a collar that is slideable on the shaft configured to hold a plurality of needles and sutures that passes through the vascular conduit. See e.g., U.S. Pat. No. 6,709,441. The suturing device may be composed of a carrier portion for inserting graft, arm portions that extend to support the graft into position, and a needle assembly adapted to retain and advance coil fasteners into engagement with the vessel wall and the graft flange to complete the anastomosis. See e.g., U.S. Pat. No. 6,709,442. The suturing device may include two oblong interlinked members that include a split bush adapted for suturing (e.g., U.S. Pat. No. 4,350,160).

One representative example of a suturing device is the HEARTFLOW device, made by Perclose-Abbott Labs, Redwood City, Calif. (see generally, U.S. Pat. Nos. 6,358,258, 6,355,050, 6,190,396, and 6,036,699, and PCT Publication No. WO 01/19257) The Nitinol U-Clip suture clip device by Coalescent Surgical (Sunnyvale, Calif.) consists of a self-closing Nitinol wire loop attached to a flexible member and a needle with a quick release mechanism. This device facilitates the construction of anastomosis by simpifying suture management and eliminating knot tying (see generally, U.S. Pat. Nos. 6,074,401 and 6,149,658, and PCT Publication Nos. WO 99/62406, WO 99/62409, WO 00/59380, WO 01/17441).

The ENCLOSE Anastomotic Assist Device (Novare Surgical Systems, Cupertino, Calif.) allows a surgeon to create a sutured anastomosis using standard suturing techniques but without the use of a partial occluding side-biting aortic clamp, avoiding aortic wall distortion (see U.S. Pat. Nos. 6,312,445 and 6,165,186).

In one aspect, automated and modified suturing methods and devices can deliver a surgical fastener (e.g., a suture or suture clip) that comprises an anti-scarring agent. In another aspect, automated and modified suturing methods and devices can deliver a vascular graft that comprises an anti-scarring agent to complete an anastomosis.

(2) Micromechanical Devices

Micromechanical devices are used to create an anastomosis and/or secure a graft vessel to the site of an anastomosis. Representative examples of micromechanical devices include staples (either penetrating or non-penetrating) and clips.

Anastomotic staple and clip devices may take a variety of forms and may be made from different types of materials. For example, staples and clips may be formed of a metal or metal alloy, such as titanium, nickel-titanium alloy, or stainless steel, or a polymeric material, such as silicone, poly(urethane), rubber, or a thermoplastic elastomer.

The polymeric material may be an absorbable or biodegradable material designed to dissolve after completion of the anastomosis. Biodegradable polymers include, for example, homopolymers and copolymers that comprise one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, Γ-decanolactone, δ-decanolactone, trimethylene carbonate, 1,4-dioxane-2-one or 1,5-dioxepan-2one.

A variety of devices for guiding staples and clips into position also have been described.

One manufacturer of non-penetrating staples for use in the creation of anastomosis is United States Surgical Corp. (Norwalk, Conn.). The VCS system (Autosuture) is an automatic stapling device that applies non-pentrating, titanium vascular clips which are usually used in an interrrupted fashion to evert tissue edges with high compressive forces. (See, e.g., U.S. Pat. Nos. 6,440,146, 6,391,039, 6,024,748, 5,833,698, 5,799,857, 5,779,718, 5,725,538, 5,725,537, 5,720,756, 5,360,154, 5,193,731, and 5,005,749 for the description of anastomotic connector devices made by U.S. Surgical).

An anastomotic clip may be composed of a shape memory material, such as nitinol, which is self-closing between an open U-shaped configuration and a closed configuration. See e.g., U.S. Pat. No. 6,641,593. The anastomotic clip may be composed of a wire having a shape memory that defines a closed configuration which may be substantially spiral-shaped and having a needle that may be releasably attached to the clip. See e.g., U.S. Pat. No. 6,551,332. Other anastomotic clips are described in, e.g., U.S. Pat. Nos. 6,461,365; and 6,514,265.

Automatic stapling devices are also made by Bypass/Ethicon, Inc. (Somerville, N.J.) and are described in, e.g., U.S. Pat. Nos. 6,193,129; 5,632,433; 5,609,285; 5,533,661; 5,439,156; 5,350,104; 5,333,773; 5,312,024; 5,292,053; 5,285,945; 5,275,322; 5,271,544; 5,271,543 and 5,205,459 and WO 03/02016. Resorbable surgical staples that include a polymer blend that is rich in glycolide (i.e., 65 to 85 weight % polymerized glycolide) are described in, e.g., U.S. Pat. Nos. 4,741,337 and 4,889,119. Surgical staples made from a blend of lactide/glycolide-copolymer and poly(p-dioxanone) are described in U.S. Pat. No. 4,646,741. Other types of stapling devices are described in, e.g., U.S. Pat. Nos. 5,234,447; 5,904,697 and 6,565,582; and U.S. Publication No. 2002/0185517A1.

In another aspect, the micromechanical device may be an anastomotic clip. For example, an anastomotic clip may be composed of a shape memory material, such as nitinol, which is self-closing between an open U-shaped configuration and a closed configuration. See e.g., U.S. Pat. No. 6,641,593. The anastomotic clip may be composed of a wire having a shape memory that defines a closed configuration which may be substantially spiral-shaped and having a needle that may be releasably attached to the clip. See e.g., U.S. Pat. No. 6,551,332. Other anastomotic clips are described in, e.g., U.S. Pat. Nos. 6,461,365; 6,187,019; and 6,514,265.

In one aspect, the present invention provides for the combination of a micromechanical anastomotic device (e.g., a staple or a clip) and an anti-scarring agent.

(3) Anastomotic Coupling Devices

Anastomotic coupling devices may be used to connect a first blood vessel to a second vessel, either with or without a graft vessel, for completion of an anastomosis. In one aspect, anastomotic coupling devices facilitate automated attachment of a graft or vessel to an aperature or orifice (e.g., in the side or at the end of a vessel) in a target vessel without the use of sutures or staples. In another aspect, the anastomotic coupling device comprises a tubular structure defining a lumen through which blood may flow (described below).

Anastomotic coupling devices that facilitate automated attachment of a graft or vessel to an aperature or orifice in a target vessel may take a variety of forms and may be made from a variety of materials. Typically, such devices are made of a biocompatible material, such as a polymer or a metal or metal alloy. For example, the device may be formed from a synthetic material, such as a fluoropolymer, such as expanded poly(tetrafluoroethylene) (ePTFE) or fluorinated ethylene propylene (FEP), a polyurethane, polyethylene, polyamide (nylon), silicone, polypropylene, polysulfone, or a polyester.

Anastomotic coupling devices may include an absorbable or biodegradable material designed to dissolve after completion of the anastomosis. Biodegradable polymers include, for example, homopolymers and copolymers that comprise one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, Γ-decanolactone, δ-decanolactone, trimethylene carbonate, 1,4-dioxane-2-one or 1,5-dioxepan-2one.

The device may include a metal or metal alloy (e.g., Nitinol, stainless steel, titanium, iron, nickel, nickel-titanium, cobalt, platinum, tungsten, tantalum, silver, gold, molybdenum, chromium, and chrome), or a combination of a metal and a polymer.

The device may be anchored to the outside of a vessel, within the tissue that surrounds the lumen of a blood vessel, and/or a portion of the device may reside within the lumen of the vessel.

In one aspect, the anastomotic coupler may be an artificially formed aperture connector that is placed in the side wall of the target vessel so that the tubular graft conduit may be extended from the target vessel. The connector may include a plurality of tissue-piercing members and retention fingers disposed in a concentric annular array which may be passed through the side wall of the tubular graft conduit for securing and retaining the graft to the connector in a fluid-tight configuration. See e.g., U.S. Pat. Nos. 6,702,829 and 6,699,256.

In another aspect, the anastomotic coupler may be in the form of a frame. For example, the frame may be configured to be deformable and scissor-shaped such that spreading members are moveable to secure a graft vessel upon insertion into a target vessel. See e.g., U.S. Pat. No. 6,179,849.

In another aspect, the anastomotic coupler may be a ring-like device that is used as an anastomotic interface between a lumen of a graft and an opening in a lumen of a target vessel. For example, the anastomotic ring may be composed of stainless steel alloy, titanium alloy, or cobalt alloy and have a flange with an expandable diameter. See e.g., U.S. Pat. No. 6,699,257. Anastomosis rings are also described in, e.g., U.S. Pat. No. 6,248,117.

In another aspect, the anastomotic coupler is resorbable. Resorbable anastomotic coupling devices may include, for example, a polymeric blend that is rich in glycolide (i.e., 65 to 85 weight % polymerized glycolide) (see, e.g., U.S. Pat. Nos. 4,741,337 and 4,889,119) or a blend of lactide/glycolide-copolymer and poly(p-dioxanone) (see, e.g., U.S. Pat. No. 4,646,741).

In another aspect, the anastomotic coupler includes a bioabsorbable, elastomeric material. Representative examples of elastomeric materials for use in resorbable devices are described in, e.g., U.S. Pat. No. 5,468,253.

In another aspect, the anastomotic coupler may be used to connect a first blood vessel to a second vessel, either with or without a graft vessel. For example, the anastomotic coupler may be a device that serves to interconnect two vessels in a side-to-side anastomosis, such as when grafting two juxtaposed cardiac vessels. The anastomotic coupler may be configured as two partially opened cylindrical segments that are interconnected along the periphery by a flow opening whereby the device may be inserted in a minimally-invasive manner which then conforms to provide pressure against the interior wall when in the original configuration such that leakage is prevented. See e.g., U.S. Pat. Nos. 6,464,709; 6,458,140 and 6,251,116 and U.S. Application Publication No. 2003/0100920A1.

In another aspect, the anastomotic coupler may also be incorporated in the design of a vascular graft to eliminate the step of attaching the interface prior to deployment. For example, the anastomotic coupler may have a leading and rear petal for dilating the vessel opening during advancement, and a base which is configured for attachment to a graft while forming a seal with the opening of the vessel. See e.g., U.S. Pat. No. 6,702,828.

In another aspect, the anastomotic coupler may be in the form of a frame. For example, the anastomotic coupler may be composed of a deformable, scissor-shaped frame with spreading members that is inserted into a target vessel. See e.g., U.S. Pat. No. 6,179,849.

In another aspect, the anastomotic coupling device may include a graft that incorporates fixation mechanisms (e.g., a collet or a grommet) at its opposite ends and a heating element to create a thermal bond between the graft and a blood vessel. (see, e.g., U.S. Pat. Nos. 6,652,544 and 6,293,955).

In another aspect, the anastomotic coupling device includes a compressible, expandable fitting for securing the ends of a bypass graft to two vessels. The fitting may be incorporated in the bypass graft design to eliminate the step of attaching the graft to the fitting prior to deployment (see, e.g., U.S. Pat. No. 6,494,889).

In another aspect, the anastomotic coupling device includes a pair of coupling disc members for joining two vessels in an end-to-end or end-to-side fashion. One of the members includes hook members, while the other member has receptor cavities aligned with the hooks for locking everted tissue of the vessels together (see, e.g., U.S. Pat. No. 4,523,592).

Representative examples of anastomotic connector devices of Bypass/Ethicon, Inc. are described in U.S. Application Publication Nos. US2002/0082625A1 and 2003/0100910A1 and U.S. Pat. Nos. 6,036,703, 6,036,700, 6,015,416, and 5,346,501.

Other anastomotic coupling devices are those described in e.g., U.S. Pat. Nos. 6,036,702; 6,508,822; 6,599,303; 6,673,084; 5,695,504; 6,569,173; 4,931,057; 5,868,763; 4,624,257; 4,917,090; 4,917,091; 5,697,943; 5,562,690; 5,454,825; 5,447,514; 5,437,684; 5,376,098; 6,652,542; 6,551,334; and 6,726,694 and U.S. Application Publication Nos. 2003/0120293A1 and 2004/0030348A1.

Anastomotic coupling devices may include proximal aortic connectors and distal coronary connectors. For example, aortic anastomotic connectors include devices such as the SYMMETRY Bypass Aortic Connector device made by St. Jude Medical, Inc. (Maple Grove, Minn.), which consists of an aortic cutter or hole punch assembly and a graft delivery system. The aortic hole punch is a cylindrical cutter with a barbed needle that provides an anchor and back pressure for the rotating cutter to core a round hole in the wall of the aorta. The graft delivery system is a radially expandable Nitinol device that holds the vein graft with small hooks which pierce throught vein graft wall. The graft is fixed to the aorta through use of an inner and outer ring of struts or flanges. This and other anastomotic connector devices by St. Jude are described in U.S. Pat. Nos. 6,309,416, 6,302,905, 6,152,937, and PCT Publication Nos. WO 00/27312 and WO 00/27311.

The CORLINK Automated Anastomotic connector device, which is produced by the CardioVations division of Ethicon, Inc. (Johnson & Johnson, Somerville, N.J.), uses a Nitinol metal alloy fastener to connect the grafted vessel to the aorta. It consists of a central cylindrical body made of interconnected elliptical arches and two sets of several pins radiating from each end. The graft is loaded into a CORLINK insertion instrument and deployed to create an anastomosis in one step.

Further examples of anastomotic coupling devices include those made by Cardica (see, U.S. Pat. Nos. 6,719,769; 6,419,681 and 6,537,287), Converge Medical (formerly Advanced Bypass Technologies), Onux Medical (see, e.g., PCT Publication No. WO 01/34037) and Ventrica, Menlo Park, Calif. (VENTRICA Magnetic Vascular Positioner) (see, e.g., U.S. Pat. Nos. 6,719,768; 6,517,558 and 6,352,543).

As described above, an anastomotic coupling device may comprise a tubular structure defining a lumen through which blood may flow. These types of devices (also referred to herein as “bypass devices”) can function as an artificial passageway or conduit for fluid communication between blood vessels and can be used to divert (i.e., shunt) blood from one part of a blood vessel (e.g., an artery) to another part of the same vessel, or to a second vessel (e.g., an artery or a vein) or to multiple vessels (e.g., a vein and an artery). In one aspect of the invention, the anastomotic device is a bypass device.

Bypass devices may be used in a variety of end-to-end and end-to-side anastomotic procedures. The bypass device may be placed into a patient where it is desired to create a pathway between two or more vascular structures, or between two different parts of the same vascular structure. For example, bypass devices may be used to create a passageway which allows blood to flow around a blood vessel, such as an artery (e.g., coronary artery, carotid artery, or artery supplying the lower limb), which has become damaged or completely or partially obstructed. Bypass devices may be used in coronary artery bypass surgery to shunt blood from an artery, such as the aorta, to a portion of a coronary artery downstream from an occlusion in the artery.

Certain types of anastomotic coupling devices are configured to join two abutting vessels. The device can further include a tubular segment to shunt blood to another vessel. These types of connectors are often used for end-to-end anastomosis if a vessel is severed or injured.

Bypass devices include at least one tubular structure having a first end and a second end, which defines a single lumen through which blood can flow, or may include more than one tubular structure, defining multiple lumens through which blood can flow. The tubular structure includes an extravascular portion and may, optionally, include an intravascular portion. The extravascular portion resides external to the adventitial tissue of a blood vessel, whereas the intravascular portion may reside within the vessel lumen or within the intimal, medial, and/or adventitial tissue.

The configuration of the tubular segment may take a variety of forms. For example, the tubular portion may be generally straight, bent or curved (e.g., L-shaped or helical), tapered, branched (e.g., bifurcated or trifurcated), or may include a network of conduits through which blood may flow. Generally, straight or bent devices have a single lumen through which blood may flow, while branched conduits (e.g., generally T-shaped and Y-shaped devices) and conduit networks (described below) have two or more lumens through which blood may flow. A tubular structure may be in the form, for example, of a hollow cylinder and may or may not include a support structure, such as a mesh or porous framework. Depending on the procedure, the device may be biodegradable or non-biodegradable; expandable or rigid; metal and/or polymeric; and/or may include a shape-memory material (e.g., Nitinol). In certain embodiments, the device may include a self-expanding stent structure.

Bypass devices typically are made of a biocompatible material. Any of the materials described above for other types of connectors may be used to make a bypass device, such as a synthetic or naturally-derived polymer, or a metal or metal alloy. For example, the device may be formed from a synthetic material, such as a fluoropolymer, such as expanded poly(tetrafluoroethylene) (ePTFE) or fluorinated ethylene propylene (FEP), a polyurethane, polyethylene, polyamide (nylon), silicone, polypropylene, polysulfone, or a polyester and/or a naturally derived material, such as collagen or a polysaccharide. The device may include a metal or metal alloy (e.g., Nitinol, stainless steel, titanium, nickel, nickel-titanium, cobalt, platinum, iron, tungsten, tantalum, silver, gold, molybdenum, chromium and chrome), or a combination of a metal and a polymer. Other types of devices include a natural graft material (e.g., autologous vessel, homologous vessel, or xenograft), or a combination of a synthetic and a natural graft material. In another aspect, the bypass device may be formed of an absorbable or biodegradable material designed to dissolve after completion of the anastomosis (e.g., polylactide, polyglycolide, and copolymers of lactide and glycolide). In yet another aspect, demineralized bone may be used to provide a pliable tubular conduit (see, e.g., U.S. Pat. No. 6,290,718).

The tubular structure(s) include a proximal end that may be configured for attachment to a proximal blood vessel and a distal end configured for attachment to a distal blood vessel. As described above, an anastomosis may be described as being either “proximal” or “distal” depending on its location relative to the vascular obstruction. The “proximal” anastomosis may be formed in a proximal blood vessel, and the “distal” anastomosis may be formed in a distal blood vessel, which may the same vessel or a different vessel than the proximal vessel. The terms “distal” and “proximal” may also be used to describe the direction that blood flows through a tubular structure from one vessel into another vessel. For example, blood may flow from a proximal vessel (e.g., the aorta) into a distal vessel, such as a coronary artery to bypass an obstruction in the coronary artery.

The tubular structure may be attached directly to a proximal or distal blood vessel. Alternatively, the bypass device may further include a graft vessel or be configured to receive a graft vessel, which can be connected to the same or a different blood vessel for completion of the anastomosis. Representative examples of graft vessels include, for example, vascular grafts or grafts used in hemodialysis applications (e.g., AV graft, AV shunt, or AV graft).

In one aspect, a tubular anastomotic coupler includes a proximal end that is attached to a proximal vessel and a distal end that is used to attach a bypass graft. The bypass graft would be secured to the distal vessel to complete the anastomosis. The direction of blood flow would be from the proximal blood vessel and into the proximal end of the tubular structure. Blood would exit through the distal end of the tubular structure and into the graft vessel.

In another aspect, the tubular anastomotic coupler includes a proximal end that is attached to a graft vessel, which is secured to the proximal blood vessel, and a distal end that is configured for attachment to a distal blood vessel. The direction of blood flow would be from the proximal vessel into the graft vessel and into the proximal end of the tubular structure. Blood would exit through the distal end of the tubular structure and into the distal vessel.

Anastomotic bypass devices may be anchored to a blood vessel in a variety of ways and may be attached to a blood vessel for the formation of an anastomosis with or without the use of sutures. Bypass devices may be attached to the outside of a blood vessel, and/or a portion of the device may be implanted into a vessel. For example, a portion of the implanted device may reside within the lumen of the vessel (i.e., endoluminally), and/or a portion of the implanted device may reside intravascularly (i.e., within the intimal, intramural, and/or adventitial tissue of the blood vessel). In one aspect, at least one of the tubular structures, or a portion thereof, may be inserted into the end of a vessel or into the side of a blood vessel. The device may be secured directly to the vessel using, for example, a fastener, such as sutures, staples, or clips and/or an adhesive. Bypass devices may include a interface to secure the conduit to a target vessel without the use of sutures. The interface may include means, such as, for example, hooks, barbs, pins, clamps, or a flange or lip for coupling the device to the site of an anastomosis.

Representative examples of anastomotic coupling devices that include at least one tubular portion include, without limitation, devices used for end-to-end anastomosis procedures (e.g., anastomotic stents and anastomotic sleeves) and end-to-side anastomosis procedures (e.g., single-lumen and multi-lumen bypass devices).

In one aspect of the invention, the anastomotic coupling device comprises a single tubular portion that may by used as a shunt to divert blood from a source vessel to a graft vessel (e.g., in an end-to-side anastomosis procedure). In one aspect, an end of the tubular portion may be connected directly or indirectly to a target vessel, as described above. The opposite end of the tubular portion may be attached to a graft vessel, where the graft vessel may be secured to a target vessel to complete the anastomosis.

The tubular portion(s) may be straight or may have a curved or bent shape (e.g., L-shaped or helical) and may be oriented orthogonally or at an angle relative to the vessel to which it is connected. In one aspect, the conduit may be secured into the site by, for example, a fastener, such as staples, clamps, or hooks, or by adhesives, radiofrequency sealing, or by other methods known to those skilled in the art.

In one aspect, the anastomotic coupling device may be, for example, a tubular metal braided graft with suture rings welded at the distal end to provide a means for securing in place to the target vessel. See e.g., U.S. Pat. No. 6,235,054. Other types of conduits that are secured into the site include, e.g., U.S. Pat. Nos. 4,368,736 and 4,366,819.

In certain types of single-lumen coupling devices, the conduit terminates in a flange that resides within the lumen of the vessel. For example, the conduit may have a tubular body with a connector which has a plurality of extensions and is configured for disposition annularly within the inside of a tubular vessel. See e.g., U.S. Pat. No. 6,660,015. In other devices, the flange may be attached into or onto the surface of the adventitial tissue of the blood vessel.

Other types of single-lumen bypass devices are described, for example, in U.S. Pat. Nos. 6,241,743; 6,428,550; 6,241,743; 6,428,550; 5,904,697; 5,290,298; 6,007,576; 6,361,559; 6,648,901, 4,931,057 and U.S. Application Publication Nos. 2004/0015180A1, 2003/0065344A1, and 2002/0116018A1.

In one aspect of the invention, the anastomotic coupling device comprises more than one lumen through which blood may travel. Multi-lumen bypass devices may include two or more tubular portions configured to interconnect multiple (two or more) blood vessels. Multi-lumen coupling devices may be used in a variety of anastomosis procedures. For example, such devices may be used in coronary artery bypass graft (CABG) surgery to divert blood from an occluded proximal vessel (e.g., an artery) into one or more target (i.e., distal) vessels (e.g., an artery or vein).

In one aspect, at least one tubular portion may by used as a shunt for diverting blood between a source vessel and a target vessel. In another aspect, the device may be configured as an interface for securing a graft vessel to a target vessel for completion of an anastomosis. Depending on the procedure, the tubular arms may be of equal length and diameter or of unequal length and diameter and may include a tubular portion(s) that is expandable and/or includes a shape-memory material (e.g., Nitinol). Furthermore, the tubular portions may be made of the same material or a different material.

In one aspect, one or more ends of a tubular portion may be inserted into the end or into the side of one or more blood vessels. In other embodiments, one or more tubular portions of the device may reside within the lumen of a blood or graft vessel. The device, optionally, may be secured to the blood vessel using a fastener or an adhesive, or another approach known to those skilled in the art.

At least one arm of the multi-lumen connector may be attached to a graft vessel. The graft vessel may be a synthetic graft, such as an ePTFE or polyester graft, or natural graft material (e.g., autologous vessel, homologous vessel, or xenograft), or a combination of a synthetic and a natural graft material. In certain embodiments, a graft vessel may be attached to an end of a tubular portion of the device, and a second graft vessel may be attached to the opposte end of the same tubular portion or to the end of another tubular portion. The graft vessel(s) may be further attached to a target vessel(s) for the completion of the anastomosis.

In one aspect, the device may include three or more tubular arms that extend from a junction site. For example,T the multi-lumen device may be generally T-shaped or Y-shaped (i.e., having two or three lumens, respectively). For example, the multi-lumen device may be a T-shaped tubular graft connector having a longitudinal member that extends into the target vessel and a second section that is exterior to the vessel which provides a connection to an alternate tubular structure. See e.g., U.S. Pat. Nos. 6,152,945 and 5,972,017. Other multi-lumen devices are described in, (see, e.g., U.S. Pat. Nos. 6,152,945; 6,451,033; 5,755,778; 5,922,022; 6,293,965; 6,517,558 and 6,626,914 and U.S. Publication No. 2004/0015180A1).

In another aspect, the device may be a tube for bypassing blood flow directly from a portion of the heart (e.g., left ventricle) to a coronary artery. For example, the device may be a hollow tube that may be partially closable by a one-way valve in response to movement of the cardiac tissue during diastole while permitting blood flow during systole (see, e.g., U.S. Pat. No. 6,641,610). The device may be an elongated rigid shunt body composed of a diversion tube having two apertures in which one may be disposed within the cyocardium of the left ventricle and the other may be disposed within the coronary artery (see, e.g., WO 00/15146 and U.S. Application Publication No. 2003/0055371A1). The device may be a valved, tubular apparatus that is L- or T-shaped which is adapted for insertion into the wall of the heart to provide blood communication from the heart to a coronary vessel (see, e.g., U.S. Pat. No. 6,123,682).

In another aspect, the device may include a network of interconnected tubular conduits. For example, the device may include two tubular portions that may be oriented generally axially or orthogonally relative to each other. See, U.S. Pat. Nos. 6,241,761 and 6,241,764. Communication between the two tubular structures may be achieved through a flow channel which facilitates blood to flow between the bores of each tube.

In another aspect, the anastomotic coupling device is a resorbable device that may be configured with two or three termini which provide a vessel interface without the need for sutures and provides a fluid communication through an intersecting lumen, such as a bypass graft or alternate vessel. See e.g., U.S. Application Publication Nos. 2002/0052572A1 and PCT Publication No. WO 02/24114A2. An anastomotic connector may also be formed of a resorbable tubular structure configured to include snap-connectors or other components for securing it to the tissue as well as hemostasis inducing sealing rings to prevent blood leakage. See e.g., U.S. Pat. No. 6,056,762. The anastomotic connector may be designed with three legs whereby two legs are adapted to be inserted within the continuous blood vessel in a contracted state and then enlarged to form a tight fit and the third leg is adapted for connecting and sealing with a third conduit. See e.g., U.S. Pat. No. 6,019,788.

An example of a commercially available multi-lumen anastomotic coupling device is the SOLEM graft connector (made by Jomed, Sweden). This device, which is described in more detail in PCT Publication No. WO 01/13820, and U.S. Pat. Nos. 6,179,848, D438618 and D429334, includes a T-shaped connector composed of Nitinol and an ePTFE graft for completion of a distal anastomosis.

In one aspect, the present invention provides for the combination of an anastomotic coupling device and an anti-scarring agent or a composition comprising an anti-scarring device. In one aspect, the anastomotic coupling device may be attached to a blood vessel for the formation of an anastomosis without the use of sutures or staples. In certain aspects, the anastomotic coupling device may comprise a tubular structure defining a lumen through which blood may flow, and an anti-scarring agent. The device may include one, two, three, or more lumens defined by one, two, three, or more tubular structures, depending on the number of vessels to be connected.

Introduction of an anastomotic connector into or onto an intramural, luminal, or adventitial portion of a blood vessel may irritate or damage the endothelial tissue of the blood vessel and/or may alter the natural hemodynamic flow through the vessel. This irritation or damage may stimulate a cascade of biological events resulting in a fibrotic response which can lead to the formation of scar tissue in the vessel. Incorporation of a therapeutic agent in accordance with the invention into or onto a portion of the device that is in direct contact with the blood vessel (e.g., a terminal portion or edge of the device) may inhibit one or more of of the scarring processes described above (e.g., smooth muscle cell proliferation, cell migration, inflammation), making the vessel less prone to the formation of intimal hyperplasia and stenosis.

Thus, in one aspect, the therapeutic agent may be associated only with the portion of the device that is in contact with the blood or endothelial tissue. For example, the anti-scarring agent may be incorporated into only an intravascular portion (i.e., that portion that resides within the lumen of the vessel or in the vessel tissue) of the device. The anti-scarring agent may be incorporated onto all or a portion of the intravascular portion of the device. In other embodiments, the coating may reside on all or a portion of an extravascular portion of the device.

The anti-scarring agent or a composition that includes an anti-scarring agent may be coated onto a portion of or onto the entire surface of the device or may be incorporated into a portion of, or into the entire the structure of, the device (e.g., either within voids, reservoirs, or divets in the device or within the material used to construct the device). In other aspects, the agent or a composition comprising the agent is impregnated into or affixed onto the device surface.

As described above, the device may include a tubular portion that is disposed within the lumen of a blood vessel. The entire tubular portion may, for example, be coated with an anti-scarring agent or a composition comprising an anti-scarring. Alternatively, only a portion of the tubular portion may include the anti-scarring agent. For example, only an external (abluminal) surface or only the interior (endoluminal) surface of the tubular portion may be coated. In other embodiments, one or both termini of the tubular portion may be coated. For example, the endoluminal and/or abluminal surface of the tubular section through which blood enters into the device (i.e., proximal end) may be coated with the anti-scarring agent or composition comprising the anti-scarring agent. In another aspect, the endoluminal and/or abluminal surface of the tubular section through which blood exits (i.e., distal end) from the device may be coated with the anti-scarring agent or composition comprising the anti-scarring agent.

In another embodiment, the anti-scarring agent or composition comprising the anti-scarring agent is associated (e.g., coated onto or incorporated into) with an anchoring member (e.g., a fastener, such as a staple or clip) that secures the device to a blood vessel.

As described above, anastomotic connector devices can include a fibrosis-inhibiting agent as a means to improve the clinical efficacy of the device. In another approach, the fibrosis-inhibiting agent can be incorporated into or onto a film or mesh that is applied in a perivascular manner to an anastomotic site (e.g., at the junction of a graft vessel and the blood vessel). These films or wraps can be used with any of the anastomotic connector devices described above and, typically, are placed around the outside of the anastomosis at the time of surgery. Representative examples of vascular wraps are described in U.S. Pat. Nos. 6,575,887 and 6,495,579 and co-pending application, entitled “Perivascular Wraps,” filed Sep. 26, 2003 (U.S. Ser. No.10/673,046). In other embodiments, the agent may be delivered to the anastomotic site in the form of a spray, paste, gel, or the like. In yet another approach, the fibrosis-inhibiting agent can be incorporated into or onto the graft vessel that is secured to the blood vessel with the connector device. Representative examples of graft vessels and methods for incorporating anti-scarring agents into and onto graft vessels, including vascular grafts and grafts used in hemodialysis applications (e.g., AV grafts, AV shunts, and AV fistulae) are described in co-pending application, entitled “Medical Implants and Anti-Scarring Agents,” filed Nov. 20, 2003 (U.S. Ser. No. 60/523,908).

It should be readily evident to one of skill in the art that a wide variety of therapeutic agents, compositions and methods can be utilized to create a variation in the compositions, devices and methods described herein without deviating from the spirit and scope of the invention.

### EXAMPLES

### Example 1

### Parylene Coating

The metallic portion of the anastomotic connector device is washed by dipping it into HPLC grade isopropanol. The cleaned anastomotic connector device is then coated with a parylene coating using a parylene coater and either di-p-xylylene or dichloro-di-p-xylylene as the coating feed material.

### Example 2

### Paclitaxel Coating—End Coating

Paclitaxel solutions are prepared by dissolving paclitaxel in 5 mL HPLC grade THF. The parylene coated anastomotic connector device ends are then dipped into the paclitaxel/THF solution. After various incubation times, the anastomotic connector devices are removed and dried in a forced air oven (50° C.). The anastomotic connector devices are then further dried in a vacuum oven overnight. The amount of paclitaxel used in each solution is varied such that the amount of paclitaxel coated onto the ends of the anastomotic connector device were in the range of 0.06 mg/mm² to 10 mg/mm².

### Example 3

### Paclitaxel Coating—Complete Coating

Paclitaxel solutions are prepared by dissolving paclitaxel in 5 mL HPLC grade THF. The entire parylene coated anastomotic connector device is then dipped into the paclitaxel/THF solution. After various incubation time, the anastomotic connector device is removed and dried in a forced air oven (50° C.). The anastomotic connector device is then further dried in a vacuum oven overnight. The amount of paclitaxel used in each solution is varied such that the amount of paclitaxel coated onto the ends of the anastomotic connector device were in the range of 0.06 mg/mm² to 10 mg/mm².

### Example 4

### Application of a Parylene Overcoat

The paclitaxel coated anastomotic connector device is placed in the Parylene coater and an additional thin layer of parylene is deposited on the paclitaxel coated anastomotic connector device (Example 2 or Example 3). The coating duration is altered such that the parylene top-coat thickness is varied such that different elution profiles of the paclitaxel can be obtained.

### Example 5

### Application of an Echogenic Coating Layer

Desmodur (BayerAG), an isocyanate pre-polymer, is dissolved in a 50:50 mixture of dimethylsulfoxide and tetrahydrofuran. The paclitaxel/parylene overcoated anastomotic connector device (Example 4) is then dipped into the pre-polymer solution. The anastomotic connector device is then removed and the coating is then partially dried at room temperature for 3 to 5 minutes. The anastomotic connector device is then immersed in a beaker of water (room temperature) for 3-5 minutes to cause the polymerization reaction to occur rapidly. An echogenic coating was formed.

### Example 6

### Paclitaxel/Polymer Coating—End Coating

5% solutions of poly(ethylene-co-vinyl acetate) {EVA} [60% vinyl acetate] are prepared using THF as the solvent. Various amounts of paclitaxel are added to each of the EVA solutions. The ends of an anastomotic connector device are dipped into the paclitaxel/EVA solution. After removing the end-coated anastomotic connector device from the solution, the coating is dried by placing the anastomotic connector device in a forced air oven (40° C.) for 3 hours. The coated anastomotic connector device is then further dried under vacuum for 24 hours. The dip coating process may be repeated to increase the amount of polymer/paclitaxel coated onto the anastomotic connector device.

### Example 7

### Paclitaxel/Polymer Coating—Outer Surface Coating

5% solutions of poly(ethylene-co-vinyl acetate) {EVA} [60% vinyl acetate] are prepared using THF as the solvent. Various amounts of paclitaxel are added to each of the EVA solutions. The outer surface of the anastomotic device (which is supported by a clamp), is coated using an airbrush sprayer. The coating is dried by placing the anastomotic connector device in a forced air oven (40° C.) for 3 hours. The spray coating process is repeated to ensure that the device is coated where the clamp had initially held the device. The coated anastomotic connector device is then further dried under vacuum for 24 hours. The coating process may be repeated to increase the amount of polymer/paclitaxel coated onto the anastomotic connector device.

### Example 8

### Paclitaxel/Polymer Coating—Inner Surface Coating

5% solutions of poly(ethylene-co-vinyl acetate) {EVA} [60% vinyl acetate] are prepared using THF as the solvent. Various amounts of paclitaxel are added to each of the EVA solutions. The inner surface of the anastomotic device is coated by gradually injecting the coating solution into the lumen of the device and gradually rotating the device at an angle such that the coating solution coated the inner surface of the device. The coating is dried by placing the anastomotic connector device in a forced air oven (40° C.) for 3 hours. The coated anastomotic connector device is then further dried under vacuum for 24 hours. The coating process may be repeated to increase the amount of polymer/paclitaxel coated onto the anastomotic connector device. The thickness of the coating is adjusted by diluting the coating solution with THF.

### Example 9

### Paclitaxel/Polymer Coating—Proximal End Coating

5% solutions of poly(ethylene-co-vinyl acetate) {EVA} [60% vinyl acetate] are prepared using THF as the solvent. Various amounts of paclitaxel are added to each of the EVA solutions. The proximal end of an anastomotic connector device is dipped into the paclitaxel/EVA solution. After removing the proximal end-coated anastomotic connector device from the solution, the coating is dried by placing the anastomotic connector device in a forced air oven (40° C.) for 3 hours. The coated anastomotic connector device is then further dried under vacuum for 24 hours. The dip coating process may be repeated to increase the amount of polymer/paclitaxel coated onto the anastomotic connector device.

### Example 10

### Paclitaxel/Polymer Coating—Distal End Coating

5% solutions of poly(ethylene-co-vinyl acetate) {EVA} [60% vinyl acetate] are prepared using THF as the solvent. Various amounts of paclitaxel are added to each of the EVA solutions. The distal end of an anastomotic connector device is dipped into the paclitaxel/EVA solution. After removing the distal end-coated anastomotic connector device from the solution, the coating is dried by placing the anastomotic connector device in a forced air oven (40° C.) for 3 hours. The coated anastomotic connector device is then further dried under vacuum for 24 hours. The dip coating process may be repeated to increase the amount of polymer/paclitaxel coated onto the anastomotic connector device.

### Example 11

### Paclitaxel-Heparin Coating—End Coating

5% solutions of poly(ethylene-co-vinyl acetate) {EVA} [60% vinyl acetate] are prepared using THF as the solvent. Various amounts of paclitaxel and a solution of tridodecyl methyl ammonium chloride-heparin complex (PolySciences) are added to each of the EVA solutions. The ends of an anastomotic connector device are dipped into the paclitaxel/EVA solution. After removing the end-coated anastomotic connector device from the solution, the coating is dried by placing the anastomotic connector device in a forced air oven (40° C.) for 3 hours. The coated anastomotic connector device is then further dried under vacuum for 24 hours.

### Example 12

### Paclitaxel—Heparin/Heparin Coating

The uncoated portions of Paclitaxel-Heparin coated anastomotic connector devices (Example 7) are dipped into a 5% EVA solution containing different amounts of a tridodecyl methyl ammonium chloride-heparin complex solution (PolySciences). After removing the end-coated anastomotic connector device from the solution, the coating is dried by placing the anastomotic connector device in a forced air oven (40° C.) for 3 hours. The coated anastomotic connector device is then further dried under vacuum for 24 hours. This provides an anastomotic connector device with a paclitaxel/heparin coating on the ends of the anastomotic connector device and a heparin coating on the remaining parts of the anastomotic connector device.

### Example 13

### Paclitaxel/Polymer Coating—End Coating

5% solutions of poly(styrene-block-isobutylene-block-styrene) (SIBS) is prepared using THF as the solvent. Various amounts of paclitaxel are added to each of the SIBS solutions. The ends of an anastomotic connector device are dipped into the paclitaxel/SIBS solution. After removing the end-coated anastomotic connector device from the solution, the coating is dried by placing the anastomotic connector device in a forced air oven (40° C.) for 3 hours. The coated anastomotic connector device is then further dried under vacuum for 24 hours. The dip coating process may be repeated to increase the amount of polymer/paclitaxel coated onto the anastomotic connector device.

### Example 14

### Paclitaxel/Polymer Coating—Echogenic Overcoat

A coated sample from example 9 is dipped into a DESMODUR (Bayer AG), an isocyanate pre-polymer, solution (50:50 mixture of dimethylsulfoxide and tetrahydrofuran). The anastomotic connector device is then removed and the coating is then partially dried at room temperature for 3 to 5 minutes. The anastomotic connector device is then immersed in a beaker of water (room temperature) for 3-5 minutes to cause the polymerization reaction to occur rapidly. An echogenic coating was formed.

### Example 15

### Polymer/Echogenic Coating

5% solutions of poly(styrene-co-isobutylene-styrene) (SIBS) is prepared using THF as the solvent. The anastomotic connector device is dipped into the SIBS solution. After removing the from the solution, the coating is dried by placing the anastomotic connector device in a forced air oven (40° C.) for 3 hours. The coated anastomotic connector device is then further dried under vacuum for 24 hours. A coated sample from example 9 is dipped into a DESMODUR (Bayer AG), an isocyanate pre-polymer, solution (50:50 mixture of dimethylsulfoxide and tetrahydrofuran). The anastomotic connector device is then removed and the coating is then partially dried at room temperature for 3 to 5 minutes. The anastomotic connector device is then immersed in a beaker of water (room temperature) for 3-5 minutes to cause the polymerization reaction to occur rapidly. The anastomotic connector device is dried under vacuum for 24 hours at room temperature. The ends of the coated anastomotic connector device are immersed into a solution of paclitaxel. The anastomotic connector device is removed and dried at 40° C. for 1 hour and then under vacuum for 24 hours.

The amount of paclitaxel absorbed by the polymeric coating can be altered by changing the paclitaxel concentration, the immersion time as well as the solvent composition of the paclitaxel solution.

### Example 16

### Paclitaxel/Siloxane Coating—End Coating

The anastomotic connector device is coated with a siloxane layer by exposing the anastomotic connector device to gaseous tetramethylcyclotetrasiloxane that is then polymerized by low energy plasma polymerization onto the anastomotic connector device surface. The thickness of the siloxane layer can be increased by increasing the polymerization time. The ends of the anastomotic connector device are then immersed into a paclitaxel/THF solution. The paclitaxel is absorbed into the siloxane coating. The anastomotic connector device is then removed from the solution and is dried for 2 hours at 40° C. in a forced air oven. The anastomotic connector device is then further dried under vacuum at room temperature for 24 hours. The amount of paclitaxel coated onto the anastomotic connector device ends can be varied by altering the concentration of the paclitaxel/THF solution as well as altering the immersion time of the anastomotic connector device ends in the paclitaxel/THF solution.

### Example 17

### Heparin Coating

The anastomotic connector device is dipped into a solution containing different amounts of a tridodecyl methyl ammonium chloride-heparin complex solution (PolySciences). After various incubation times, the anastomotic connector device is removed and dried in a forced air oven (50° C.). The anastomotic connector device is then further dried in a vacuum oven overnight.

### Example 18

### Parylene/Heparin Coating

The parylene coated anastomotic connector device (Example 1) was dipped into a solution containing different amounts of a tridodecyl methyl ammonium chloride-heparin complex solution (PolySciences). After various incubation times, the anastomotic connector device is removed and dried in a forced air oven (50° C.). The anastomotic connector device is then further dried in a vacuum oven overnight.

### Example 19

### Heparin/Polymer Coating

A 5% solution of poly(styrene-co-isobutylene-styrene) (SIBS) is prepared using THF as the solvent. Various amounts of a tridodecyl methyl ammonium chloride-heparin complex solution (PolySciences) are added to each of the SIBS solutions. The anastomotic connector device is dipped into the paclitaxel/SIBS solution. After removing the anastomotic connector device from the solution, the coating is dried by placing the anastomotic connector device in a forced air oven (40° C.) for 3 hours. The coated anastomotic connector device is then further dried under vacuum for 24 hours. The dip coating process may be repeated to increase the amount of polymer/heparin coated onto the anastomotic connector device.

### Example 20

### Spray-Coated Devices

2% solutions poly(styrene-co-isobutylene-styrene) (SIBS) are prepared using THF as the solvent. Various amounts of paclitaxel are added to each solution. An anastomotic connector device is held with a pair of tweezers and is then spray coated with one of the paclitaxel/polymer solutions using an airbrush. The device is then air-dried. The device is then held in a new location using the tweezers and a second coat of paclitaxel/polymer is applied. The device is air-dried and is then dried under vacuum overnight. The total amount of paclitaxel coated onto the device can be altered by changing the paclitaxel content in the solution as well as by increasing the number of coating applied.

### Example 21

### Screening Assay For Assessing the Effect of Mitoxantrone on Cell Proliferation

Fibroblasts at 70-90% confluency are trypsinized, replated at 600 cells/well in media in 96-well plates and allowed to attachment overnight. Mitoxantrone is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing fibroblasts and mitoxantrone are incubated at 37° C. for 72 hours (In vitro toxicol. (1990) 3:219; Biotech. Histochem. (1993) 68: 29; Anal. Biochem. (1993) 213:426).

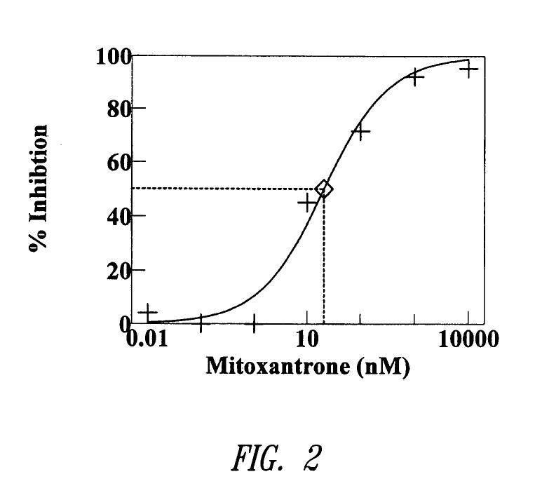

To terminate the assay, the media is removed by gentle aspiration. A 1/400 dilution of CYQUANT 400× GR dye indicator (Molecular Probes; Eugene, Ore.) is added to 1×0 Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Inhibitory concentration of 50% (IC₅₀) is determined by taking the average of triplicate wells and comparing average relative fluorescence units to the DMSO control. An average of n=4 replicate experiments is used to determine IC₅₀ values. The results of the assay are shown in FIG. 2.

### Example 22

### Screening Assay For Assessing the Effect of Motoxantrone on Nitric Oxide Production By Macrophages

The murine macrophage cell line RAW 264.7 is trypsinized to remove cells from flasks and plated in individual wells of a 6-well plate. Approximately 2×10⁶ cells are plated in 2 mL of media containing 5% heat-inactivated fetal bovine serum (FBS). RAW 264.7 cells are incubated at 37° C. for 1.5 hours to allow adherence to plastic. Mitoxantrone is prepared in DMSO at a concentration of 10⁻² M and serially diluted 10-fold to give a range of stock concentrations (10⁻⁸ M to 10⁻² M). Media is then removed and cells are incubated in 1 ng/mL of recombinant murine IFNΓ and 5 ng/mL of LPS with or without mitoxantrone in fresh media containing 5% FBS. Mitoxantrone is added to cells by directly adding mitoxantrone DMSO stock solutions, prepared earlier, at a 1/1000 dilution, to each well. Plates containing IFNΓ, LPS plus or minus mitoxantrone are incubated at 37° C. for 24 hours (Chem. Ber. (1879) 12:426; J. AOAC (1977) 60-594; Ann. Rev. Biochem. (1994)63:175).

At the end of the 24 hour period, supernatants are collected from the cells and assayed for the production of nitrites. Each sample is tested in triplicate by aliquoting 50 μL of supernatant in a 96-well plate and adding 50 μL of Greiss Reagent A (0.5 g sulfanilamide, 1.5 mL H₃PO₄, 48.5 mL ddH₂O) and 50 μL of Greiss Reagent B (0.05 g N-(1-Naphthyl)-ethylenediamine, 1.5 mL H₃PO₄, 48.5 mL ddH₂O). Optical density is read immediately on microplate spectrophotometer at 562 nm absorbance. Absorbance over triplicate wells is averaged after subtracting background and concentration values are obtained from the nitrite standard curve (1 μM to 2 mM). Inhibitory concentration of 50% (IC₅₀) is determined by comparing average nitrite concentration to the positive control (cell stimulated with IFNΓ and LPS). An average of n=4 replicate experiments is used to determine IC₅₀ values for mitoxantrone. The results of the assay are shown in FIG. 2.

### Example 23

### Screening Assay For Assessing the Effect of Bay11-7082 on TNF-α Production By Macrophages

The human macrophage cell line, THP-1 is plated in a 12 well plate such that each well contains 1×10⁶ cells in 2 mL of media containing 10% FCS. Opsonized zymosan is prepared by resuspending 20 mg of zymosan A in 2 mL of ddH₂O and homogenizing until a uniform suspension is obtained. Homogenized zymosan is pelleted at 250 g and resuspended in 4 mL of human serum for a final concentration of 5 mg/mL. and incubated in a 37° C. water bath for 20 minutes to enable opsonization. Bay 11-7082 is prepared in DMSO at a concentration of 10⁻² M and serially diluted 10-fold to give a range of stock concentrations (10⁻⁸ M to 10⁻² M) (J. Immunol. (2000) 165: 411-418; J. Immunol. (2000) 164: 4804-4811; J. Immunol Meth. (2000) 235 (1-2): 33-40).

THP-1 cells are stimulated to produce TNFA by the addition of 1 mg/mL opsonized zymosan. Bay 11-7082 is added to THP-1 cells by directly adding DMSO stock solutions, prepared earlier, at a 1/1000 dilution, to each well. Each drug concentration is tested in triplicate wells. Plates are incubated at 37° C. for 24 hours.

After a 24 hour stimulation, supernatants are collected to quantify TNFα production. TNFα concentrations in the supernatants are determined by ELISA using recombinant human TNFα to obtain a standard curve. A 96-well MaxiSorb plate is coated with 100 μL of anti-human TNFα Capture Antibody diluted in Coating Buffer (0.1M Sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% TWEEN-20). Plates are blocked for 1 hour at room temperature with 200 μL/well of Assay Diluent (PBS, 10% and 1/16; (b) recombinant human TNFα is prepared at 500 pg/mL and serially diluted to yield as standard curve of 7.8 pg/mL to 500 pg/mL. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human TNFα detection antibody+avidin-HRP) for 1 hour at room temperature. Following this incubation, the plates are washed 7 times and 100 μL of Substrate Solution (Tetramethylbenzidine, H₂O₂) is added to plates and incubated for 30 minutes at room temperature. Stop Solution (2 N H₂SO₄) is then added to the wells and a yellow colour reaction is read at 450 nm with λ correction at 570 nm. Mean absorbance is determined from triplicate data readings and the mean background is subtracted. TNFα concentration values are obtained from the standard curve. Inhibitory concentration of 50% (IC₅₀) is determined by comparing average TNFα concentration to the positive control (THP-1 cells stimulated with opsonized zymosan). An average of n=4 replicate experiments is used to determine IC₅₀ values for Bay 11-7082.

### Example 24

### Surgical Adhesions Model to Assess Fibrosis Inhibiting Agents

The rabbit uterine horn model is used to assess the anti-fibrotic capacity of formulations in vivo. Mature New Zealand White (NZW) female rabbits are placed under general anesthetic. Using aseptic precautions, the abdomen is opened in two layers at the midline to expose the uterus. Both uterine horns are lifted out of the abdominal cavity and assessed for size on the French Scale of catheters. Horns between #8 and #14 on the French Scale (2.5-4.5 mm diameter) are deemed suitable for this model. Both uterine horns and the opposing peritoneal wall are abraded with a #10 scalpel blade at a 45° angle over an area 2.5 cm in length and 0.4 cm in width until punctuate bleeding is observed. Abraded surfaces are tamponaded until bleeding stops. The individual horns are then opposed to the peritoneal wall and secured by two sutures placed 2 mm beyond the edges of the abraded area. The formulation is applied and the abdomen is closed in three layers. After 14 days, animals are evaluated post mortem with the extent and severity of adhesions being scored both quantitatively and qualitatively.

### Example 25

### Evaluation of Paclitaxel Containing Mesh On Intimal Hyperplasia Development IN a Rat Balloon Injury Carotid Artery Model

A rat balloon injury carotid artery model was used to demonstrate the efficacy of a paclitaxel containing mesh system on the development of intimal hyperplasia fourteen days following placement.

Control Group

Wistar rats weighing 400-500 g were anesthetized with 1.5% halothane in oxygen and the left external carotid artery was exposed. An A 2 French Fogarty balloon embolectomy catheter (Baxter, Irvine, Calif.) was advanced through an arteriotomy in the external carotid artery down the left common carotid artery to the aorta. The balloon was inflated with enough saline to generate slight resistance (approximately 0.02 ml) and it was withdrawn with a twisting motion to the carotid bifurcation. The balloon was then deflated and the procedure repeated twice more. This technique produced distension of the arterial wall and denudation of the endothelium. The external carotid artery was ligated after removal of the catheter. The right common carotid artery was not injured and was used as a control.

Local Perivascular Paclitaxel Treatment

Immediately after injury of the left common carotid artery, a 1 cm long distal segment of the artery was exposed and treated with a 1×1 cm paclitaxel-containing mesh. The wound was then closed the animals were kept for 14 days.

Histology and immunohistochemistry

At the time of sacrifice, the animals were euthanized with carbon dioxide and pressure perfused at 100 mmHg with 10% phosphate buffered formaldehyde for 15 minutes. Both carotid arteries were harvested and left overnight in fixative. The fixed arteries were processed and embedded in paraffin wax. Serial cross-sections were cut at 3 μm thickness every 2 mm within and outside the implant region of the injured left carotid artery and at corresponding levels in the control right carotid artery. Cross-sections were stained with Mayer's hematoxylin-and-eosin for cell count and with Movat's pentachrome stains for morphometry analysis and for extracellular matrix composition assessment.

Results

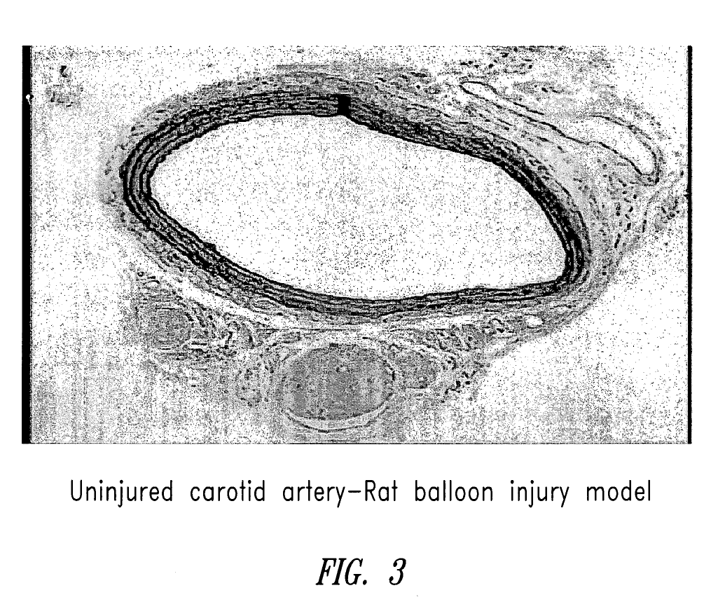

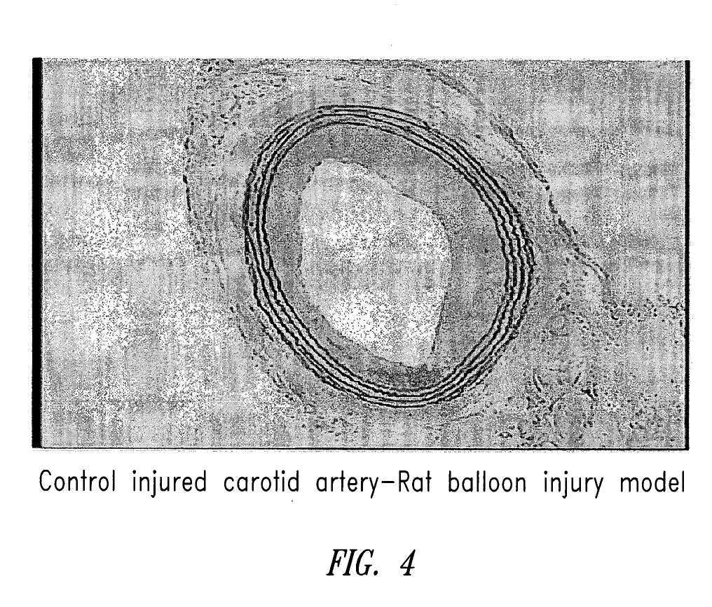

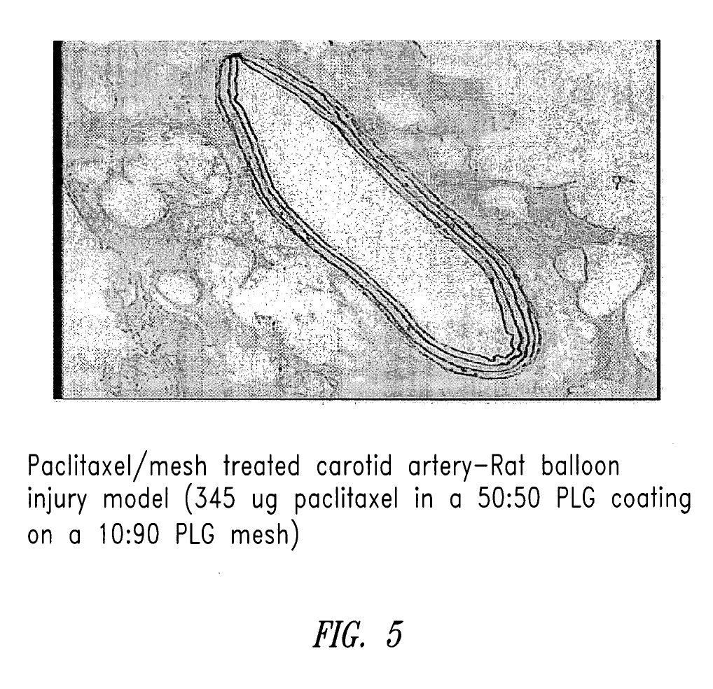

From FIGS. 3-5, it is evident that the perivascular delivery of paclitaxel using the paclitaxel.mesh formulation resulted is a dramatic reduction in intimal hyperplasia.

### Example 26

### Effect of Paclitaxel and Other Anti-Microtubule Agents On Marix Metalloproteinase Production

### A. MATERIALS AMD METHODS

1. IL-1 Stimulated AP-1 Transcriptional Activity Is Inhibited By Paclitaxel

Chondrocytes were transfected with constructs containing an AP-1 driven CAT reporter gene, and stimulated with IL-1, IL-1 (50 ng/ml) was added and incubated for 24 hours in the absence and presence of paclitaxel at various concentrations. Paclitaxel treatment decreased CAT activity in a concentration dependent manner (mean±SD). The data noted with an asterisk (*) have significance compared with IL-1-induced CAT activity according to a t-test, P<0.05. The results shown are representative of three independent experiments.

2. Effect of Paclitaxel On IL-1 Induced AP-1 DNA Binding Activity, AP-1 DNA

Binding activity was assayed with a radiolabeled human AP-1 sequence probe and gel mobility shift assay. Extracts from chondrocytes untreated or treated with various amounts of paclitaxel (10⁻⁷ to 10⁻⁵ M) followed by IL-1β (20 ng/ml) were incubated with excess probe on ice for 30 minutes, followed by non-denaturing gel electrophoresis. The “com” lane contains excess unlabeled AP-1 oligonucleotide. The results shown are representative of three independent experiments.

3. Effect of Paclitaxel On IL-1 Induced MMP-1 and MMP-3 mRNA Expression

Cells were treated with paclitaxel at various concentrations (10⁻⁷ to 10⁻⁵ M) for 24 hours. Then, treated with IL-1β (20 ng/ml) for additional 18 hours in the presence of paclitaxel. Total RNA was isolated, and the MMP-1 mRNA levels were determined by Northern blot analysis. The blots were subsequently stripped and reprobed with ³²P-radiolabeled rat GAPDH cDNA, which was used as a housekeeping gene. The results shown are representative of four independent experiments. Quantitation of collagenase-1 and stromelysin-expression mRNA levels. The MMP-1 and MMP-3 expression levels were normalized with GAPDH.

4. Effect of Other Anti-Microtubules On Collagenase Expression

Primary chondrocyte cultures were freshly isolated from calf cartilage. The cells were plated at 2.5×10⁶ per ml in 100×20 mm culture dishes and incubated in Ham's F12 medium containing 5% FBS overnight at 37° C. The cells were starved in serum-free medium overnight and then treated with anti-microtubule agents at various concentrations for 6 hours. IL-1 (20 ng/ml) was then added to each plate and the plates incubated for an additional 18 hours. Total RNA was isolated by the acidified guanidine isothiocyanate method and subjected to electrophoresis on a denatured gel. Denatured RNA samples (15 μg) were analyzed by gel electrophoresis in a 1 % denatured gel, transferred to a nylon membrane and hydridized with the ³²P-labeled collagenase cDNA probe. ³²P-labeled glyceraldehyde phosphate dehydrase (GAPDH) cDNA as an internal standard to ensure roughly equal loading. The exposed films were scanned and quantitatively analyzed with ImageQuant.

### B. RESULTS

1. Promoters On the Family of Matrix Metalloproteinases

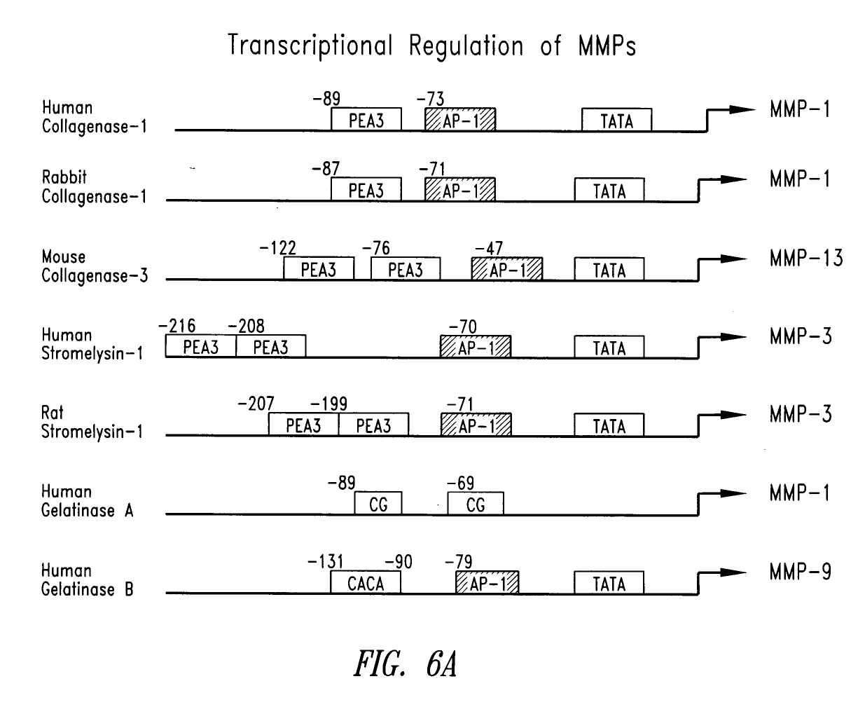

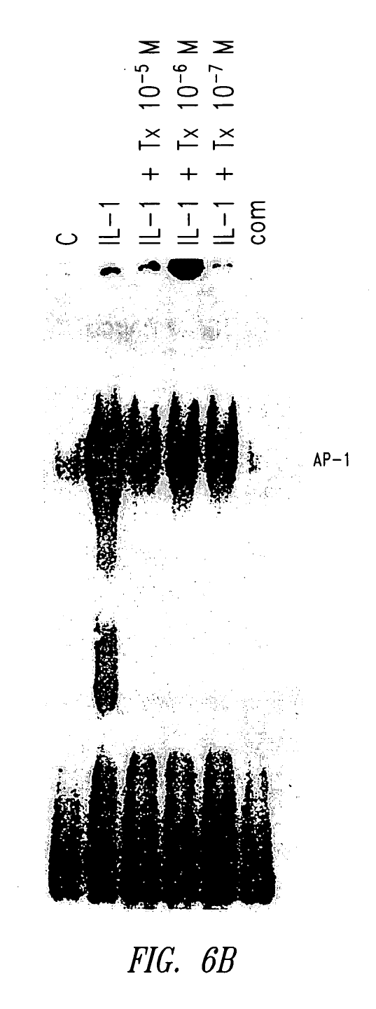

FIG. 6A shows that all matrix metalloproteinases contained the transcriptional elements AP-1 and PEA-3 with the exception of Gelatinase B. It has been well established that expression of matrix metalloproteinases such as collagenases and stromelysins are dependent on the activation of the transcription factors AP-1. Thus inhibitors of AP-1 would inhibit the expression of matrix metalloproteinases.

2. Effect of Paclitaxel On AP-1 Transcriptional Activity

As demonstrated in FIG. 6B, IL-1 stimulated AP-1 transcriptional activity 5-fold. Pretreatment of transiently transfected chondrocytes with paclitaxel reduced IL-1 induced AP-1 reporter gene CAT activity. Thus, IL-1 induced AP-1 activity was reduced in chondrocytes by paclitaxel in a concentration dependent manner (10⁻⁷ to 10⁻⁵ M). These data demonstrated that paclitaxel was a potent inhibitor of AP-1 activity in chondrocytes.

3. Effect of Paclitaxel On AP-1 DNA Binding Activity

To confirm that paclitaxel inhibition of AP-1 activity was not due to nonspecific effects, the effect of paclitaxel on IL-1 induced AP-1 binding to oligonucleotides using chondrocyte nuclear lysates was examined. As shown in FIG. 19C, IL-1 induced binding activity decreased in lysates from chondrocyte which have been pretreated with paclitaxel at concentration ₁₀⁻⁷ to 10⁻⁵ M for 24 hours. Paclitaxel inhibition of AP-1 transcriptional activity closely correlated with the decrease in AP-1 binding to DNA.

4. Effect of Paclitaxel On Collagenase and Stromelysin Expression

Since paclitaxel was a potent inhibitor of AP-1 activity, the effect of paclitaxel or IL-1 induced collagenase and stromelysin expression, two important matrix metalloproteinases involved in inflammatory diseases was examined. Briefly, as shown in FIG. 20, IL-1 induction increases collagenase and stromelysin mRNA levels in chondrocytes. Pretreatment of chondrocytes with paclitaxel for 24 hours significantly reduced the levels of collagenase and stromelysin mRNA. At 10⁻⁵ M paclitaxel, there was complete inhibition. The results show that paclitaxel completely inhibited the expression of two matrix metalloproteinases at concentrations similar to which it inhibits AP-1 activity.

5. Effect of Other Anti-Microtubules On Collagenase Expression

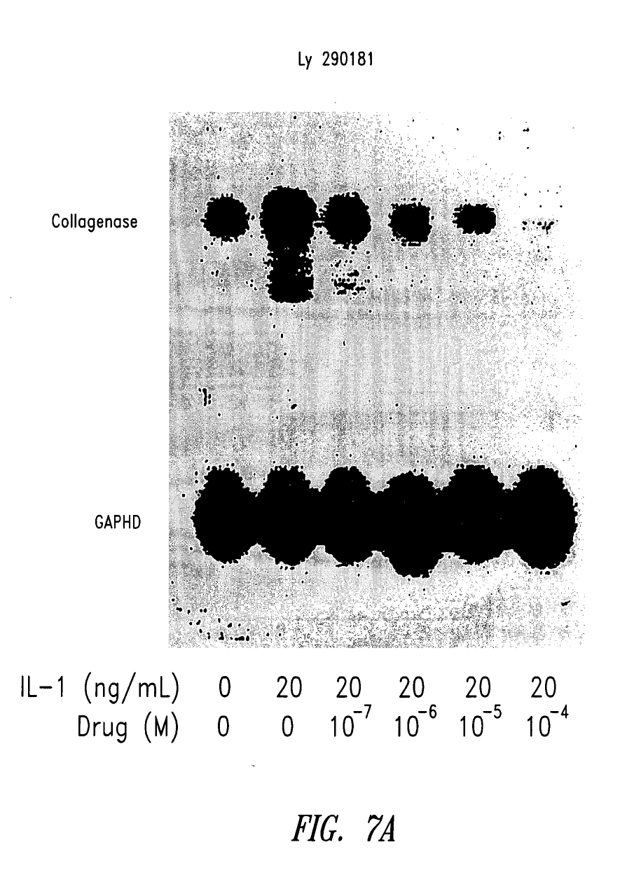

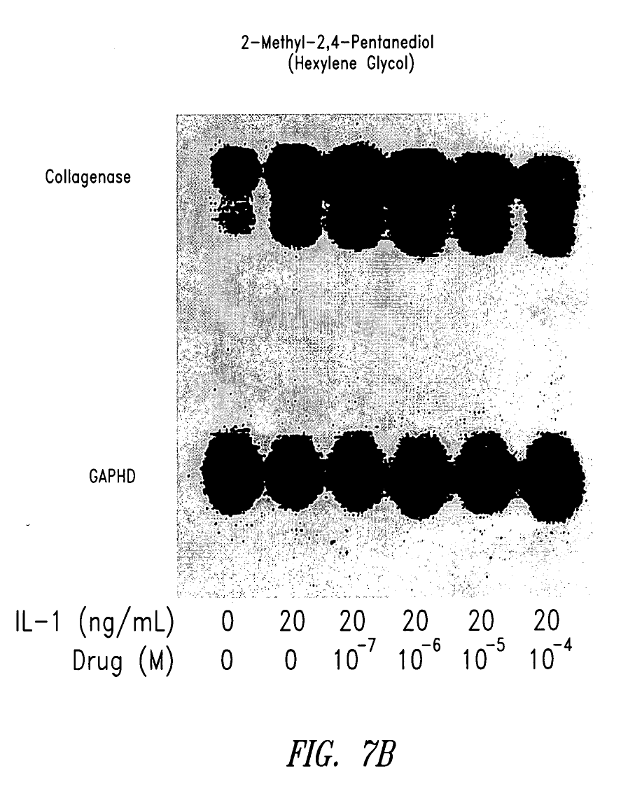

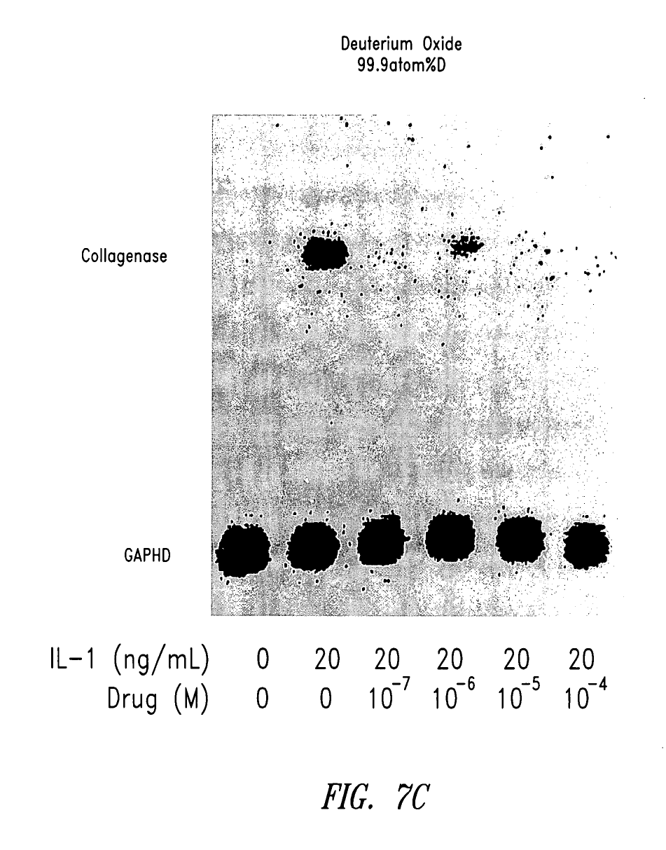

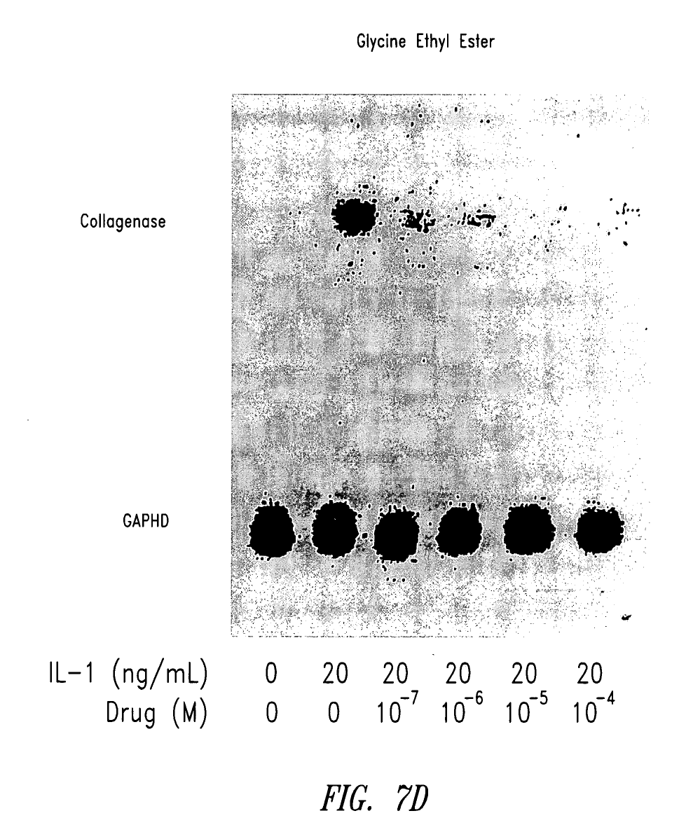

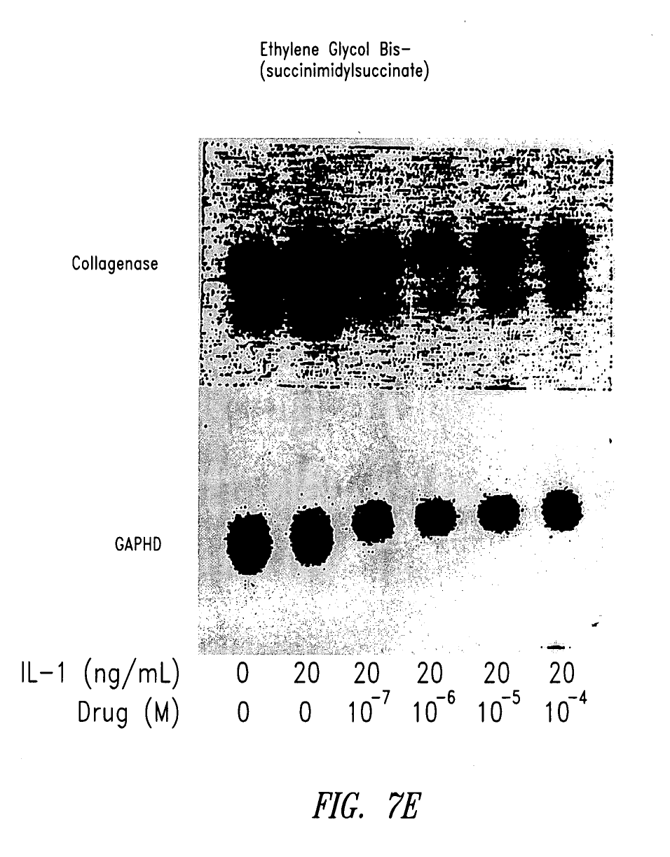

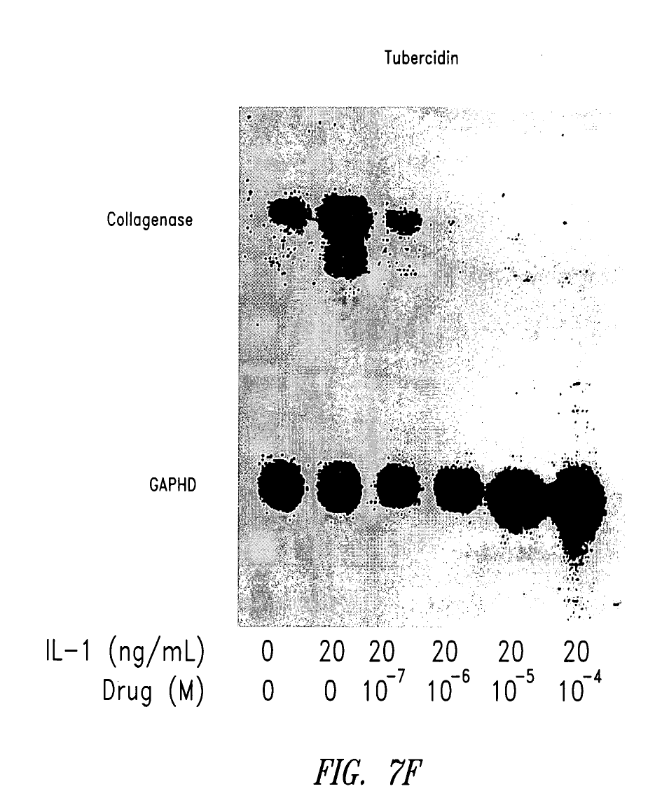

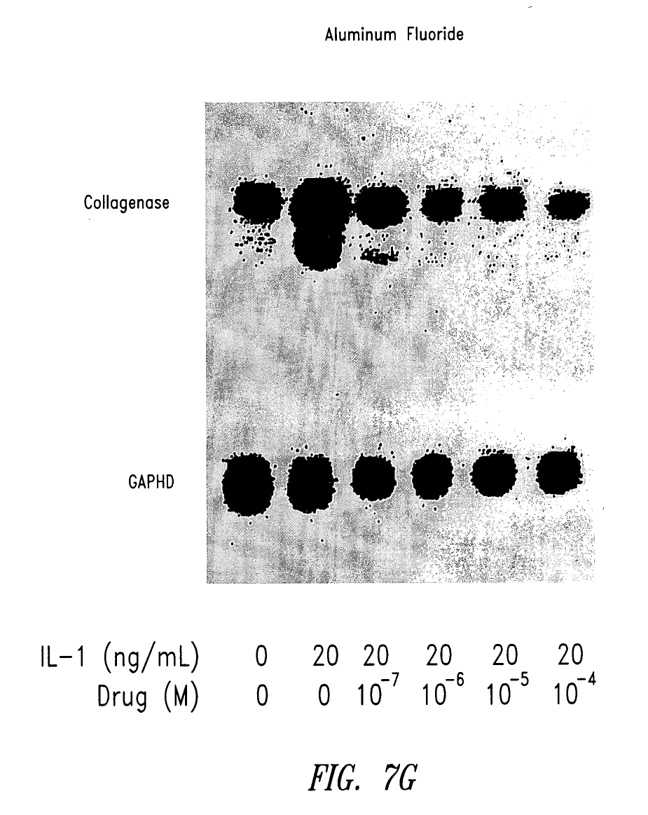

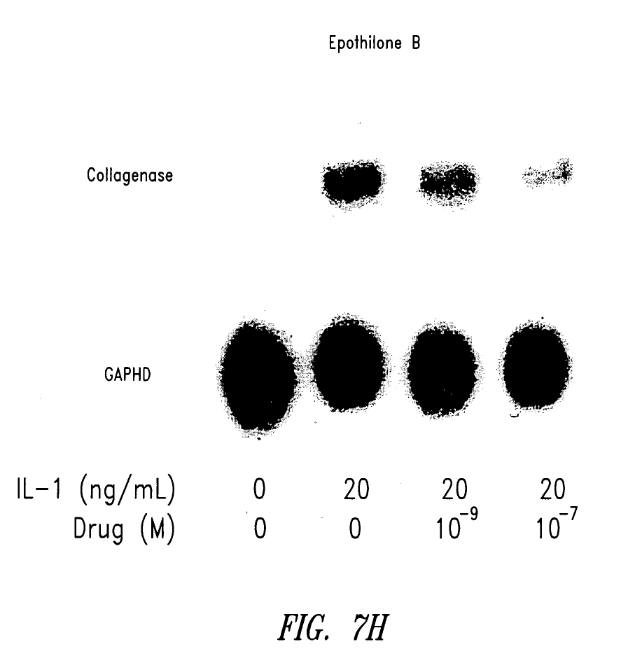

FIGS. 7A-H demonstrate that anti-microtubule agents inhibited collagenase expression. Expression of collagenase was stimulated by the addition of IL-1 which is a proinflammatory cytokine. Pre-incubation of chondrocytes with various anti-microtubule agents, specifically LY290181, hexylene glycol, deuterium oxide, glycine ethyl ester, AIF₃, tubercidin epothilone, and ethylene glycol bis-(succinimidylsuccinate), all prevented IL-1-induced collagenase expression at concentrations as low as 1×10⁻⁷ M.

### C. DISCUSSION

Paclitaxel was capable of inhibiting collagenase and stromelysin expression in vitro at concentrations of 10⁻⁶ M. Since this inhibition can be explained by the inhibition of AP-1 activity, a required step in the induction of all matrix metalloproteinases with the exception of gelatinase B, it is expected that paclitaxel would inhibit other matrix metalloproteinases which are AP-1 dependent. The levels of these matrix metalloproteinases are elevated in all inflammatory diseases and play a principle role in matrix degradation, cellular migration and proliferation, and angiogenesis. Thus, paclitaxel inhibition of expression of matrix metalloproteinases such as collagenase and stromelysin will have a beneficial effect in inflammatory diseases.

In addition to paclitaxel's inhibitory effect on collagenase expression, LY290181, hexylene glycol, deuterium oxide, glycine ethyl ester, AIF₃, tubercidin epothilone, and ethylene glycol bis-(succinimidylsuccinate), all prevented IL-1-induced collagenase expression at concentrations as low as 1×10⁻⁷ M. Thus, anti-microtubule agents are capable of inhibiting the AP-1 pathway at varying concentrations.

### Example 27

### Inhibition of Angiogenisis By Paclitaxel

A. Chick Chorioallantoic Membrane (“CAM”) Assays

Fertilized, domestic chick embryos were incubated for 3 days prior to shell-less culturing. In this procedure, the egg contents were emptied by removing the shell located around the air space. The interior shell membrane was then severed and the opposite end of the shell was perforated to allow the contents of the egg to gently slide out from the blunted end. The egg contents were emptied into round-bottom sterilized glass bowls and covered with petri dish covers. These were then placed into an incubator at 90% relative humidity and 3% CO₂ and incubated for 3 days.

Paclitaxel (Sigma, St. Louis, Mich.) was mixed at concentrations of 0.25, 0.5, 1, 5, 10, 30 μg per 10 ul aliquot of 0.5% aqueous methylcellulose. Since paclitaxel is insoluble in water, glass beads were used to produce fine particles. Ten microliter aliquots of this solution were dried on parafilm for 1 hour forming disks 2 mm in diameter. The dried disks containing paclitaxel were then carefully placed at the growing edge of each CAM at day 6 of incubation. Controls were obtained by placing paclitaxel-free methylcellulose disks on the CAMs over the same time course. After a 2 day exposure (day 8 of incubation) the vasculature was examined with the aid of a stereomicroscope. Liposyn II, a white opaque solution, was injected into the CAM to increase the visibility of the vascular details. The vasculature of unstained, living embryos were imaged using a Zeiss stereomicroscope which was interfaced with a video camera (Dage-MTI Inc., Michigan City, Ind.). These video signals were then displayed at 160× magnification and captured using an image analysis system (Vidas, Kontron; Etching, Germany). Image negatives were then made on a graphics recorder (Model 3000; Matrix Instruments, Orangeburg, N.Y.).

The membranes of the 8 day-old shell-less embryo were flooded with 2% glutaraldehyde in 0.1M sodium cacodylate buffer; additional fixative was injected under the CAM. After 10 minutes in situ, the CAM was removed and placed into fresh fixative for 2 hours at room temperature. The tissue was then washed overnight in cacodylate buffer containing 6% sucrose. The areas of interest were posffixed in 1 % osmium tetroxide for 1.5 hours at 4° C. The tissues were then dehydrated in a graded series of ethanols, solvent exchanged with propylene oxide, and embedded in Spurr resin. Thin sections were cut with a diamond knife, placed on copper grids, stained, and examined in a Joel 1200EX electron microscope. Similarly, 0.5 mm sections were cut and stained with toluene blue for light microscopy.

At day 11 of development, chick embryos were used for the corrosion casting technique. Mercox resin (Ted Pella, Inc., Redding, Calif.) was injected into the CAM vasculature using a 30-gauge hypodermic needle. The casting material consisted of 2.5 grams of Mercox CL-2B polymer and 0.05 grams of catalyst (55% benzoyl peroxide) having a 5 minute polymerization time. After injection, the plastic was allowed to sit in situ for an hour at room temperature and then overnight in an oven at 65° C. The CAM was then placed in 50% aqueous solution of sodium hydroxide to digest all organic components. The plastic casts were washed extensively in distilled water, air-dried, coated with gold/palladium, and viewed with the Philips 501 B scanning electron microscope.

Results of the assay are as follows. At day 6 of incubation, the embryo is centrally positioned to a radially expanding network of blood vessels; the CAM develops adjacent to the embryo. These growing vessels lie close to the surface and are readily visible making this system an idealized model for the study of angiogenesis. Living, unstained capillary networks of the CAM can be imaged noninvasively with a stereomicroscope.

Transverse sections through the CAM show an outer ectoderm consisting of a double cell layer, a broader mesodermal layer containing capillaries which lie subjacent to the ectoderm, adventitial cells, and an inner, single endodermal cell layer. At the electron microscopic level, the typical structural details of the CAM capillaries are demonstrated. Typically, these vessels lie in close association with the inner cell layer of ectoderm.

After 48 hours exposure to paclitaxel at concentrations of 0.25, 0.5, 1, 5, 10, or 30 μg, each CAM was examined under living conditions with a stereomicroscope equipped with a video/computer interface in order to evaluate the effects on angiogenesis. This imaging setup was used at a magnification of 160× which permitted the direct visualization of blood cells within the capillaries; thereby blood flow in areas of interest could be easily assessed and recorded. For this study, the inhibition of angiogenesis was defined as an area of the CAM (measuring 2-6 mm in diameter) lacking a capillary network and vascular blood flow. Throughout the experiments, avascular zones were assessed on a 4 point avascular gradient (Table 1). This scale represents the degree of overall inhibition with maximal inhibition represented as a 3 on the avascular gradient scale. Paclitaxel was very consistent and induced a maximal avascular zone (6 mm in diameter or a 3 on the avascular gradient scale) within 48 hours depending on its concentration.

TABLE 1
AVASCULAR GRADIENT

0 -- normal vascularity
1 -- lacking some microvascular movement
2* -- small avascular zone approximately 2 mm in diameter
3* -- avascularity extending beyond the disk (6 mm in diameter)
*indicates a positive antiangiogenesis response

The dose-dependent, experimental data of the effects of paclitaxel at different concentrations are shown in Table 2.

TABLE 2

| Agent | Delivery Vehicle | Concentration | Inhibition/n |
|---|---|---|---|
| paclitaxel | methylcellulose (10 ul) | 0.25 ug | 2/11 |
| | methylcellulose (10 ul) | 0.5 ug | 6/11 |
| | methylcellulose (10 ul) | 1 ug | 6/15 |
| | methylcellulose (10 ul) | 5 ug | 20/27 |
| | methylcellulose (10 ul) | 10 ug | 16/21 |
| | methylcellulose (10 ul) | 30 ug | 31/31 |

Typical paclitaxel-treated CAMs are also shown with the transparent methylcellulose disk centrally positioned over the avascular zone measuring 6 mm in diameter. At a slightly higher magnification, the periphery of such avascular zones is clearly evident; the surrounding functional vessels were often redirected away from the source of paclitaxel. Such angular redirecting of blood flow was never observed under normal conditions. Another feature of the effects of paclitaxel was the formation of blood islands within the avascular zone representing the aggregation of blood cells.

In summary, this study demonstrated that 48 hours after paclitaxel application to the CAM, angiogenesis was inhibited. The blood vessel inhibition formed an avascular zone which was represented by three transitional phases of paclitaxel's effect. The central, most affected area of the avascular zone contained disrupted capillaries with extravasated red blood cells; this indicated that intercellular junctions between endothelial cells were absent. The cells of the endoderm and ectoderm maintained their intercellular junctions and therefore these germ layers remained intact; however, they were slightly thickened. As the normal vascular area was approached, the blood vessels retained their junctional complexes and therefore also remained intact. At the periphery of the paclitaxel-treated zone, further blood vessel growth was inhibited which was evident by the typical redirecting or “elbowing” effect of the blood vessels.

### Example 28

### Screening Assay For Assessing the Effect of Paclitaxel On Smooth Muscle Cell Migration

Primary human smooth muscle cells are starved of serum in smooth muscle cell basal media containing insulin and human basic fibroblast growth factor (bFGF) for 16 hours prior to the assay. For the migration assay, cells are trypsinized to remove cells from flasks, washed with migration media and diluted to a concentration of 2-2.5×105 cells/mL in migration media. Migration media consists of phenol red free Dulbecco's Modified Eagle Medium (DMEM) containing 0.35% human serum albumin. A 100 μL volume of smooth muscle cells (approximately 20,000-25,000 cells) is added to the top of a Boyden chamber assembly (Chemicon QCM Chemotaxis 96-well migration plate). To the bottom wells, the chemotactic agent, recombinant human platelet derived growth factor (rhPDGF-BB) is added at a concentration of 10 ng/mL in a total volume of 150 μL. Paclitaxel is prepared in DMSO at a concentration of 10⁻² M and serially diluted 10-fold to give a range of stock concentrations (10⁻⁸ M to 10⁻² M). Paclitaxel is added to cells by directly adding paclitaxel DMSO stock solutions, prepared earlier, at a 1/1000 dilution, to the cells in the top chamber. Plates are incubated for 4 hours to allow cell migration.

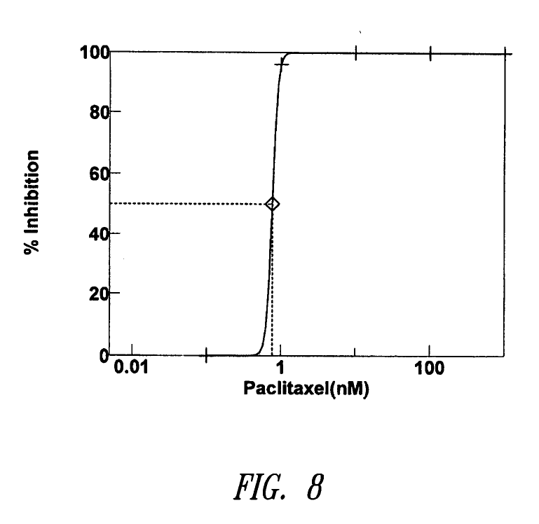

At the end of the 4 hour period, cells in the top chamber are discarded and the smooth muscle cells attached to the underside of the filter are detached for 30 minutes at 37° C. in Cell Detachment Solution (Chemicon). Dislodged cells are lysed in lysis buffer containing the DNA binding CyQuant GR dye and incubated at room temperature for 15 minutes. Fluorescence is read in a fluorescence microplate reader at ˜480 nm excitation wavelength and ˜520 nm emission maxima. Relative fluorescence units from triplicate wells are averaged after subtracting background fluorescence (control chamber without chemoattractant) and average number of cells migrating is obtained from a standard curve of smooth muscle cells serially diluted from 25,000 cells/well down to 98 cells/well. Inhibitory concentration of 50% (IC₅₀) is determined by comparing the average number of cells migrating in the presence of paclitaxel to the positive control (smooth muscle cell chemotaxis in reponse to rhPDGF-BB). See, FIG. 8. References: Biotechniques (2000) 29: 81; J. Immunol Methods (2001) 254: 85

### Example 29

### Screening Assay For Assessong the Effect of Geldanamycin On IL-1β Production By Macrophages

The human macrophage cell line, THP-1 is plated in a 12 well plate such that each well contains 1×10⁶ cells in 2 mL of media containing 10% FCS. Opsonized zymosan is prepared by resuspending 20 mg of zymosan A in 2 mL of ddH₂O and homogenizing until a uniform suspension is obtained. Homogenized zymosan is pelleted at 250 g and resuspended in 4 mL of human serum for a final concentration of 5 mg/mL. and incubated in a 37° C. water bath for 20 minutes to enable opsonization. Geldanamycin is prepared in DMSO at a concentration of 10⁻² M and serially diluted 10-fold to give a range of stock concentrations (10⁻⁸ M to 10⁻² M).

THP-1 cells are stimulated to produce IL-1β by the addition of 1 mg/mL opsonized zymosan. Geldanamycin is added to THP-1 cells by directly adding DMSO stock solutions, prepared earlier, at a 1/1000 dilution, to each well. Each drug concentration is tested in triplicate wells. Plates are incubated at 37° C. for 24 hours.

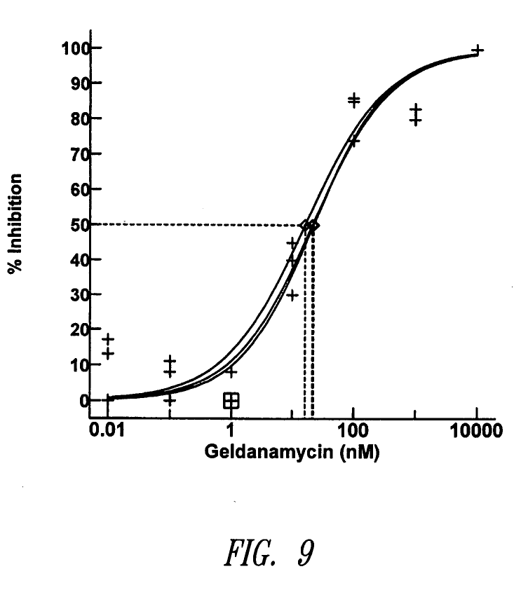

After a 24 hour stimulation, supernatants are collected to quantify IL-1β production. IL-1β concentrations in the supernatants are determined by ELISA using recombinant human IL-1β to obtain a standard curve. A 96-well MaxiSorb plate is coated with 100 μL of anti-human IL-1β Capture Antibody diluted in Coating Buffer (0.1M Sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% Tween-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted ¼ and ⅛; (b) recombinant human IL-1β is prepared at 1000 pg/mL and serially diluted to yield as standard curve of 15.6 pg/mL to 1000 pg/mL. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human IL-1β detection antibody+avidin-HRP) for 1 hour at room temperature. Following this incubation, the plates are washed 7 times and 100 μL of Substrate Solution (Tetramethylbenzidine, H₂O₂) is added to plates and incubated for 30 minutes at room temperature. Stop Solution (2 N H₂SO₄) is then added to the wells and a yellow colour reaction is read at 450 nm with λ correction at 570 nm. Mean absorbance is determined from triplicate data readings and the mean background is subtracted. IL-1β concentration values are obtained from the standard curve. Inhibitory concentration of 50% (IC₅₀) is determined by comparing average IL-1β concentration to the positive control (THP-1 cells stimulated with opsonized zymosan). An average of n=4 replicate experiments is used to determine IC₅₀ values for Geldanamycin. See, FIG. 9. References: J. Immunol. (2000) 165: 411-418; J. Immunol. (2000) 164: 4804-4811; J. Immunol Meth. (2000) 235 (1-2): 33-40

### Example 30

### Screening Assay For Assessing the Effect of Geldanamycin On IL-8 Production By Macrophages

The human macrophage cell line, THP-1 is plated in a 12 well plate such that each well contains 1×10⁶ cells in 2 mL of media containing 10% FCS. Opsonized zymosan is prepared by resuspending 20 mg of zymosan A in 2 mL of ddH₂O and homogenizing until a uniform suspension is obtained. Homogenized zymosan is pelleted at 250 g and resuspended in 4 mL of human serum for a final concentration of 5 mg/mL. and incubated in a 37° C. water bath for 20 minutes to enable opsonization. Geldanamycin is prepared in DMSO at a concentration of 10⁻² M and serially diluted 10-fold to give a range of stock concentrations (10⁻⁸ M to 10⁻² M).

THP-1 cells are stimulated to produce IL-8 by the addition of 1 mg/mL opsonized zymosan. Geldanamycin is added to THP-1 cells by directly adding DMSO stock solutions, prepared earlier, at a 1/1000 dilution, to each well. Each drug concentration is tested in triplicate wells. Plates are incubated at 37° C. for 24 hours.

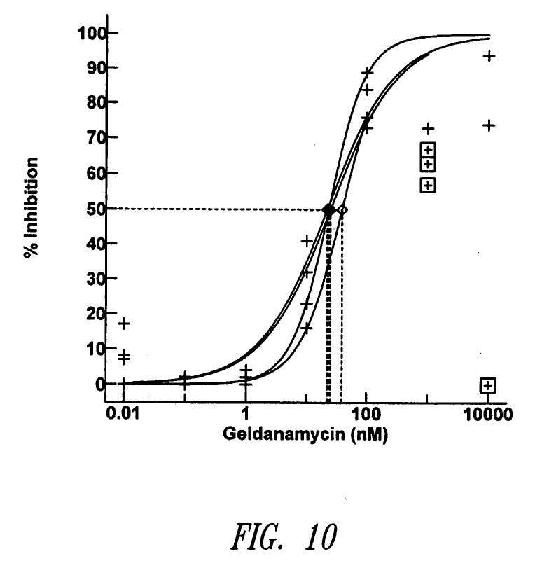

After a 24 hour stimulation, supernatants are collected to quantify IL-8 production. IL-8 concentrations in the supernatants are determined by ELISA using recombinant human IL-8 to obtain a standard curve. A 96-well MaxiSorb plate is coated with 100 μL of anti-human IL-8 Capture Antibody diluted in Coating Buffer (0.1M Sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% Tween-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted 1/100 and 1/100; (b) recombinant human IL-8 is prepared at 200 pg/mL and serially diluted to yield as standard curve of 3.1 pg/mL to 200 pg/mL. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human IL-8 detection antibody+avidin-HRP) for 1 hour at room temperature. Following this incubation, the plates are washed 7 times and 100 μL of Substrate Solution (Tetramethylbenzidine, H₂O₂) is added to plates and incubated for 30 minutes at room temperature. Stop Solution (2 N H₂SO₄) is then added to the wells and a yellow colour reaction is read at 450 nm with λ correction at 570 nm. Mean absorbance is determined from triplicate data readings and the mean background is subtracted. IL-8 concentration values are obtained from the standard curve. Inhibitory concentration of 50% (IC₅₀) is determined by comparing average IL-8 concentration to the positive control (THP-1 cells stimulated with opsonized zymosan). An average of n=4 replicate experiments is used to determine IC₅₀ values for Geldanamycin. See, FIG. 10. References: J. Immunol. (2000) 165: 411-418; J. Immunol. (2000) 164: 4804-4811; J. Immunol Meth. (2000) 235 (1-2): 33-40

### Example 31

### Screening Assay For Assessing the Effect of Geldanamycin On MCP-1 Production By Macrophages

The human macrophage cell line, THP-1 is plated in a 12 well plate such that each well contains 1×10⁶ cells in 2 mL of media containing 10% FCS. Opsonized zymosan is prepared by resuspending 20 mg of zymosan A in 2 mL of ddH₂O and homogenizing until a uniform suspension is obtained. Homogenized zymosan is pelleted at 250 g and resuspended in 4 mL of human serum for a final concentration of 5 mg/mL. and incubated in a 37° C. water bath for 20 minutes to enable opsonization. Geldanamycin is prepared in DMSO at a concentration of 10⁻² M and serially diluted 10-fold to give a range of stock concentrations (10⁻⁸ M to 10⁻² M).

THP-1 cells are stimulated to produce MCP-1 by the addition of 1 mg/mL opsonized zymosan. Geldanamycin is added to THP-1 cells by directly adding DMSO stock solutions, prepared earlier, at a 1/1000 dilution, to each well. Each drug concentration is tested in triplicate wells. Plates are incubated at 37° C. for 24 hours.

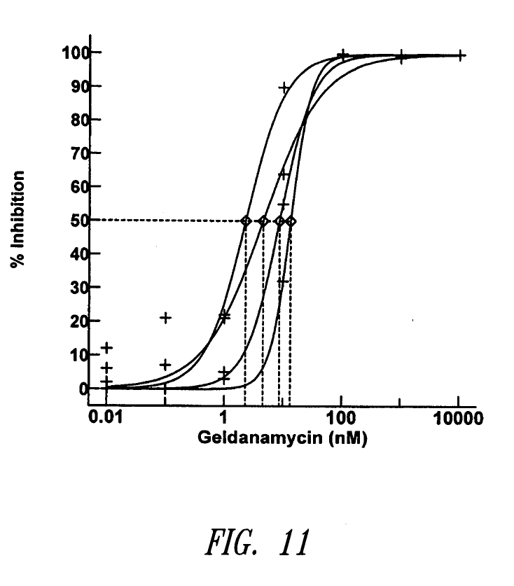

After a 24 hour stimulation, supernatants are collected to quantify MCP-1 production. MCP-1 concentrations in the supernatants are determined by ELISA using recombinant human MCP-1 to obtain a standard curve. A 96-well MaxiSorb plate is coated with 100 μL of anti-human MCP-1 Capture Antibody diluted in Coating Buffer (0.1M Sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% Tween-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted 1/100 and 1/1000; (b) recombinant human MCP-1 is prepared at 500 pg/mL and serially diluted to yield as standard curve of 7.8 μg/mL to 500 pg/mL. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human MCP-1 detection antibody+avidin-HRP) for 1 hour at room temperature. Following this incubation, the plates are washed 7 times and 100 μL of Substrate Solution (Tetramethylbenzidine, H₂O₂) is added to plates and incubated for 30 minutes at room temperature. Stop Solution (2 N H₂SO₄) is then added to the wells and a yellow colour reaction is read at 450 nm with λ correction at 570 nm. Mean absorbance is determined from triplicate data readings and the mean background is subtracted. MCP-1 concentration values are obtained from the standard curve. Inhibitory concentration of 50% (IC₅₀) is determined by comparing average MCP-1 concentration to the positive control (THP-1 cells stimulated with opsonized zymosan). An average of n=4 replicate experiments is used to determine IC₅₀ values for Geldanamycin. See, FIG. 11. References: J. Immunol. (2000) 165: 411-418; J. Immunol. (2000) 164: 4804-4811; J. Immunol Meth. (2000) 235 (1-2): 33-40.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. An anastomotic coupling device comprising an anastomotic coupling device and an anti-scarring agent, wherein the agent is releasable from the device at a therapeutically effective concentration that inhibits stenosis.

2. An anastomotic coupling device of claim 1 further comprising a polymeric carrier in admixture with the anti-scarring agent.

3. The anastomotic coupling device of claim 1 wherein the device comprises a tubular structure defining a lumen through which blood may flow, the tubular structure having a first end and a second end.

4. The anastomotic coupling device of claim 3 wherein the device comprises more than one tubular structure.

5. The anastomotic coupling device of claim 4 wherein the device comprises two tubular structures.

6. The anastomotic coupling device of claim 4 wherein the device comprises three three tubular structures.

7. The anastomotic coupling device of claim 1 or 2 wherein the device comprises an extravascular portion.

8. The anastomotic coupling device of claim 7 wherein the device further comprises an intravascular portion.

9. The anastomotic coupling device of claim 8 wherein intravascular portion is configured to reside within a lumen of a blood vessel.

10. The anastomotic coupling device of claim 8 wherein intravascular portion is configured to reside within a tissue of a blood vessel.

11. The anastomotic coupling device of claim 1 wherein the device is constructed from materials comprising a metal or metal alloy.

12. The anastomotic coupling device of claim 11 wherein the metal or metal alloy is selected from the group consisting of stainless steel, titanium, platinum, tantalum, zirconium, chromium, cobalt chromium, cobalt-chromium-molybdenum alloys, cobalt-chromium-tungsten alloys, nickel-titanium alloys, and titanium-aluminum alloys.

13. The anastomotic coupling device of claim 1 or 2 wherein the device comprises a crystalline or amorphous ceramic material.

14. The anastomotic coupling device of claim 13 wherein the ceramic material is selected from the group consisting of aluminum oxides, titanium oxides, hydroxyapatite, yttrium oxides, silicon oxides, and zirconium oxides.

15. The anastomotic coupling device of claim 1 or 2 wherein the device is constructed from materials comprising a polymer.

16. The anastomotic coupling device of claim 15 wherein the polymer is selected from the group consisting of silicones, polyurethanes, polysulfone, epoxies, polytetrafluoroethylene, fluorinated ethylene propylene, polycarbonate, polyamides, polyesters, polyarylene-etherketone, polyarylene ethers, polyimides, polyvinylchloride, polyoxymethylene, and poly ether ether ketone.

17. The anastomotic coupling device of claim 1 or 2 wherein the device is constructed from materials comprising a composite material.

18. The anastomotic coupling device of claim 17 wherein the composite material comprises a metal or metal alloy and a ceramic material.

19. The anastomotic coupling device of claim 18 wherein the composite material further comprises a polymeric material.

20. The anastomotic coupling device of claim 17 wherein the composite material comprises a metal or metal alloy and a polymeric material.

21. The anastomotic coupling device of claim 17 wherein the composite material comprises a ceramic material and a polymeric material.

22. The anastomotic coupling device of claim 1 or 2 wherein the device is configured to attach a first artery to a second artery, an artery to a vein, or a first vein to a second vein.

23. The anastomotic coupling device of claim 1 or 2 wherein the device is configured to join a blood vessel to a graft vessel.

24. The anastomotic coupling device of claim 23 wherein the blood vessel is an artery or a vein.

25. The anastomotic coupling device of claim 1 further comprising a drug-releasing layer, wherein the drug-releasing layer comprises the agent.

26. The anastomotic coupling device of claim 25 wherein the drug-releasing layer resides on all or a portion of the device.

27. The anastomotic coupling device of claim 7 further comprising a drug-releasing layer, the drug-releasing layer comprising the anti-scarring agent, wherein the drug-releasing layer resides on a surface of the extravascular portion of the device.

28. The anastomotic coupling device of claim 7 further comprising a drug-releasing layer, the drug-releasing layer comprising the anti-scarring agent, wherein the drug-releasing layer resides on a surface of the intravascular portion of the device.

29. The anastomotic coupling device of claim 3 further comprising a drug-releasing layer, the drug-releasing layer comprising the anti-scarring agent.

30. The anastomotic coupling device of claim 29 wherein the drug-releasing layer resides on an abluminal surface of the tubular structure.

31. The anastomotic coupling device of claim 29 wherein the drug-releasing layer resides on an endoluminal surface of the tubular structure.

32. The anastomotic coupling device of claim 29 wherein the drug-releasing layer resides on the first end or the second end of the tubular structure.

33. The anastomotic coupling device of claim 29 wherein the drug-releasing layer resides on the first end and the second end of the tubular structure.

34. The anastomotic coupling device of claim 25 wherein the device comprises two or more drug-releasing layers.

35. The anastomotic coupling device of claim 34 wherein the device comprises two drug-releasing layers, wherein the first drug-releasing layer has a first composition and the second drug-releasing layer has a second composition.

36. The anastomotic coupling device of claim 35 wherein the first composition and the second composition are different.

37. The anastomotic coupling device of claim 25 wherein the drug-releasing layer comprises a biodegradable polymer.

38. The anastomotic coupling device of claim 37 wherein the biodegradable polymer is formed from one or more monomers selected from the group consisting of lactide, glycolide, lactic acid, ε-caprolactone, trimethylene carbonate, 1,4-dioxan-2-one, 1,5-dioxepan-2-one, 1,4-dioxepan-2-one, hydroxyvalerate, and hydroxybutyrate.

39. The device of claim 37 wherein the biodegradable polymer carrier comprises a poly(lactic acid), copolymer of lactic acid and glycolic acid, a copolymer of lactide and glycolide, a copolymer of D,L-lactide and glycolide, copolymer of lactide and ε-caprolactone, or poly(caprolactone).

40. The anastomotic coupling device of claim 25 wherein the drug-releasing layer comprises hyaluronic acid, chitosan, or sodium alginate.

41. The anastomotic coupling device of claim 25 wherein the drug-releasing layer comprises a non-polymeric carrier.

42. The device of claim 41 wherein the non-polymeric carrier is selected from the group consisting of sucrose acetate isobutyrate, calcium stearate, sucrose ester, sucrose oleate, and a wax.

43. The device of claim 42 wherein the wax is paraffin wax or microcrystalline wax.

44. The anastomotic coupling device of claim 25 wherein the drug-releasing layer comprises a non-biodegradable polymer.

45. The anastomotic coupling device of claim 44 wherein non-biodegradable polymer is selected from the group consisting of cellulose esters, polyurethanes, polyvinyl-pyrrolidones, acrylate polymers and copolymers, methacrylate polymers and copolymers, and polyvinylpyrrolidone-vinylacetate copolymers, and blends thereof.

46. The anastomotic coupling device of claim 25 wherein the device further comprises a bonding layer disposed on a device surface, wherein the bonding layer bonds the drug-releasing layer to the surface of the device.

47. The anastomotic coupling device of claim 46 wherein the composition of the first bonding layer is different than the composition of the drug-releasing layer.

48. The anastomotic coupling device of claim 1 or 2 further comprising a bonding layer disposed on a surface of the device and a surface layer, wherein the agent resides between the bonding layer and the surface layer.

49. The anastomotic coupling device of claim 46, wherein the first bonding layer comprises a polymer selected from the group consisting of polyethylene-acrylic acid copolymers, epoxy resins, acrylate polymers and copolymers having functional groups comprising carboxyl, hydroxyl, acetyl, and amino functionalities.

50. The anastomotic coupling device of claim 46 wherein the device further comprises a second bonding layer, wherein the second bonding layer is disposed between the first bonding layer and the drug releasing layer, wherein the second bonding layer bonds the drug-releasing layer to the first bonding layer.

51. The anastomotic coupling device of claim 50 wherein the second bonding layer has a composition that is different than the composition of the first bonding layer or the composition of the drug-releasing layer.

52. The anastomotic coupling device of claim 50 wherein the second bonding layer comprises a polymer selected from the group consisting of polyurethanes, polycarbonate urethanes, alkyl and aryl urethanes, polymers and copolymers of ethyl and butyl methacrylate, and polyvinylacetal polymers.

53. The anastomotic coupling device of claim 52 wherein the polyvinylacetal polymer is a polyvinylbutyral.

54. The anastomotic coupling device of claim 1 or 2 wherein the device further comprises a graft vessel.

55. The anastomotic coupling device of claim 54 wherein the graft vessel comprises an anti-scarring agent.

56. The anastomotic coupling device of claim 1 wherein a surface of the device comprises about 0.01 μg /mm² to about 10 μg/mm² of anti-scarring agent.

57. The anastomotic coupling device of claim 1 wherein a surface of the device comprises about 0.25 μg/mm²- 5 μg/mm² of anti-scarring agent.

58. The anastomotic coupling device of of claim 1 or 2 wherein the agent inhibits proliferation of smooth muscle cells, or fibroblasts, or a combination thereof.

59. The anastomotic coupling device of claim 1 or 2 wherein the agent further inhibits inflammation, cell migration, or angiogenesis, or a combination thereof.

60. The anastomotic coupling device of claim 1 or 2 wherein the agent inhibits inflammation.

61. The anastomotic coupling device of claim 1 or 2 wherein the agent inhibits cell migration.

62. The anastomotic coupling device of claim 1 or 2 wherein the agent inhibits angiogenesis.

63. The anastomotic coupling device of claim 1 or 2 wherein the agent is not sirolimus or an analogue or derivative thereof.

64. A device of claim 1 or 2 wherein the agent is a cell cycle inhibitor.

65. The anastomotic coupling device of claim 64 wherein the cell cycle inhibitor is an anthracycline.

66. The anastomotic coupling device of claim 65 wherein the anthracycline is doxorubicin, mitoxantrone, or an analogue or derivative thereof.

67. The anastomotic coupling device of claim 64 wherein a surface of the device comprises about 0.01 μg/mm² to about 100 μg/mm² cell cycle inhibitor.

68. The anastomotic coupling device of claim 64 wherein a surface of the device comprises about 0.1 μg/mm² to about 10 μg/mm² cell cycle inhibitor.

69. The anastomotic coupling device of claim 64 wherein the cell cycle inhibitor is an anti-microtubule agent.

70. The anastomotic coupling device of claim 67 wherein the anti-microtubule agent is a taxane.

71. The anastomotic coupling device of claim 68 wherein the taxane is paclitaxel, docetaxel, or a derivative or analogue thereof.

72. The anastomotic coupling device of claim 71 wherein a surface of the device comprises about 0.1 μg/mm² to about 10 μg/mm² paclitaxel.

73. The anastomotic coupling device of claim 71 wherein a surface of the device comprises about 0.25 μg/mm² to about 5 μg/mm² paclitaxel.

74. The anastomotic coupling device of claim 64 wherein the cell cycle inhibitor is a podophyllotoxin.

75. The anastomotic coupling device of claim 74 wherein the podophyllotoxin is etoposide or a derivative or analogue thereof.

76. The anastomotic coupling device of claim 75 wherein a surface of the device comprises about 0.1 μg/mm² to about 10 μg/mm² etoposide.

77. The anastomotic coupling device of claim 75 wherein a surface of the device comprises about 0.25 μg/mm² to about 5 μg/mm² etoposide.

78. The anastomotic coupling device of claim 64 wherein the cell cycle inhibitor is a topoisomerase inhibitor.

79. The anastomotic coupling device of claim 78 wherein the topoisomerase inhibitor is camptothecin.

80. The anastomotic coupling device of claim 79 wherein a surface of the device comprises about 0.1 μg/mm² to about 10 μg/mm² camptothecin.

81. The anastomotic coupling device of claim 79 wherein a surface of the device comprises about 0.25 μg/mm² to about 5 μg/mm² of camptothecin.

82. The anastomotic coupling device of claim 1 or 2 wherein the agent is a heat shock protein 90 antagonist.

83. The anastomotic coupling device of claim 82 wherein the heat shock protein 90 antagonist is geldanamycin, anisomycin, or an analogue or derivative thereof.

84. The anastomotic coupling device of claim 83 wherein a surface of the device comprises about 0.1 μg/mm² to about 10 μg/mm² geldanamycin.

85. The anastomotic coupling device of claim 83 wherein a surface of the device comprises about 0.25 μg/mm² to about 5 μg/mm² geldanamycin.

86. The anastomotic coupling device of claim 1 or 2 wherein the agent is a pyrolidine antibiotic.

87. The anastomotic coupling device of claim 86 wherein the pyrolidine antibiotic is anisomycin.

88. The anastomotic coupling device of claim 87 wherein a surface of the device comprises about 0.1 μg/mm² to about 10 μg/mm² anisomycin.

89. The anastomotic coupling device of claim 87 wherein a surface of the device comprises about 0.25 μg/mm² to about 5 μg/mm² anisomycin.

90. The anastomotic coupling device of claim 1 or 2 wherein the agent is an immunomodulator.

91. The anastomotic coupling device of claim 90 wherein the immunomodulator is sirolimus or an analogue or derivative thereof.

92. The anastomotic coupling device of claim 91 wherein the sirolimus analogue is everolimus, biolimus, or tacrolimus.

93. The anastomotic coupling device of claim 91 wherein a surface of the device comprises about 0.1 μg/mm² to about 100 μg/mm² sirolimus.

94. The anastomotic coupling device of claim 91 wherein a surface of the device comprises about 0.5 μg/mm² to about 10 μg/mm² of sirolimus.

95. The anastomotic coupling device of claim 92 wherein a surface of the device comprises about 0.1 μg/mm² to about 100 μg/mm² everolimus.

96. The anastomotic coupling device of claim 92 wherein a surface of the device comprises about 0.3 μg/mm² to about 10 μg/mm² everolimus.

97. The anastomotic coupling device of claim 1 or 2 wherein the agent is an inosine monophosphate dehydrogenase (IMPDH) inhibitor.

98. The anastomotic coupling device of claim 97 wherein the IMPDH inhibitor is mycophenolic acid.

99. The anastomotic coupling device of claim 98 wherein a surface of the device comprises about 0.1 μg/mm² to about 50 μg/mm² mycophenolic acid.

100. The anastomotic coupling device of claim 98 wherein a surface of the device comprises about 0.25 μg/mm² to about 25 μg/mm² mycophenolic acid.

101. The anastomotic coupling device of claim 1 or 2 wherein the device further comprises an anti-thrombogenic, an anti-platelet agent, or a combination thereof.

102. The anastomotic coupling device of claim 101 wherein the anti-thrombogenic agent is heparin or a derivative thereof.

103. The anastomotic coupling device of claim 102 wherein the heparin derivative is benzalkonium heparinate or tridodecylmethylammonium heparinate.

104. A method for inhibiting scarring at an anastomotic site comprising implanting a device according to any one of claims 1 to 103 into an animal host.

105. A method for inhibiting scarring comprising placing an anastomotic coupling device into an animal host, wherein the anastomotic coupling device releases an anti-scarring agent or an agent that inhibits stenosis.

106. A method for creating a pathway between two vascular structures, or between two different parts of the same vascular structure, comprising introducing an anastomotic coupling device into a patient where it is desired to create a pathway between two vascular structures, or between two different parts of the same vascular structure, wherein the anastomotic coupling device is a device of any one of claims 1 to 103.

107. A method for creating a pathway between an artery and a vein, comprising introducing an anastomotic coupling device into a patient where it is desired to create a pathway between an artery and a vein, wherein the anastomotic coupling device is a device of any one of claims 1 to 103.

108. A method for creating a pathway between a first artery and a second artery, comprising introducing an anastomotic coupling device into a patient where it is desired to create a pathway between a first artery and a second artery, wherein the anastomotic coupling device is a device of any one of claims 1 to 103.

109. A method for treating or preventing intimal hyperplasia at an anastomotic site, comprising delivering to an anastomotic site an anastomotic coupling device of any one of claims 1 to 103.

110. The method of claim 109 wherein the anastomotic site is an arterial anastomosis.

111. The method of claim 109 wherein the anastomotic site is a venous anastomosis.

112. The method of claim 109 wherein the device is delivered to an extravascular portion of the anastomotic site.

113. The method of claim 109 wherein the device is delivered to an intravascular portion of the anastomotic site.

114. An anastomotic staple device which is adapted to release an anti-scarring agent.

115. An anastomotic clip device which is adapted to release an anti-scarring agent.

116. A suture device which is adapted to release an anti-scarring agent.

117. The device of any one of claims 114-116 wherein the anti-scarring agent is selected from the group consisting of paclitaxel, camptothecin, mycophenolic acid, mitoxantrone, doxorubicin, etoposide, geldanamycin, and analogues or derivatives thereof.
